(19) United States
(12) Patent Application Publication
(10) Pub. No.: US 2014/0297199 A1
(43) Pub. Date: Oct. 2, 2014

(54) DRUG SCREENING METHOD AND USES THEREOF

(71) Applicant: COLD SPRING HARBOR LABORATORY, Cold Spring Harbor, NY (US)

(72) Inventor: Pavel Osten, New York, NY (US)

(73) Assignee: COLD SPRING HARBOR LABORATORY, Cold Spring Harbor, NY (US)

(21) Appl. No.: 14/356,975

(22) PCT Filed: Nov. 9, 2012
(86) PCT No.: PCT/US2012/064440

§ 371 Date: May 8, 2014

Related U.S. Application Data
(60) Provisional application No. 61/558,877, filed on Nov. 11, 2011.

(51) Int. Cl. G01N 33/50

(52) U.S. Cl. CPC G01N 33/5008; G01N 33/5014
USPC 702/19; 435/7.1

(57) ABSTRACT

Described herein are methods of screening drugs in a non-human animal using high resolution technology leading to generation of pharmacomaps. Further described herein are methods of predicting the therapeutic benefit and/or toxicity of drug candidate compounds. In specific embodiments, provided herein are methods of predicting the clinical effects of a test drug based on comparison of the pharmacomap of the test drug to the pharmacomap of one or more reference drugs with known clinical outcomes.

This application claims the benefit of U.S. provisional application No. 61/558,877 filed Nov. 11, 2011, which is incorporated by reference herein in its entirety.

### 1. INTRODUCTION

Described herein are methods of screening drugs in a non-human animal using high resolution technology leading to generation of pharmacomaps. Further described herein are methods of predicting the therapeutic benefit and/or toxicity of drug candidate compounds. In specific embodiments, provided herein are methods of predicting the clinical effects of a test drug based on comparison of the pharmacomap of the test drug to the pharmacomap of one or more reference drugs with known clinical outcomes.

### 2. BACKGROUND

The development of new drugs or medications often involves assessment of effects of the drugs or medications on animals. Laboratory animals, such as mice, are used for obtaining experimental data so that subsequent tests on human beings may be safely performed. For example, a new drug may activate certain brain cells of laboratory mice, which can be identified using immediate early genes (IEGs), such as c-fos and Arc (activity regulated cytoskeletal protein). Traditionally, IEG induction is detected by labor-intensive and error-prone techniques, such as in situ hybridization or immunohistochemistry, followed by visual inspection, markup and scoring of a subset of brain regions by human observer.

### 3. SUMMARY

In one aspect, provided herein is a method of generating a pharmacomap, comprising: (a) administering a compound to a non-human animal; and (b) imaging a tissue of the non-human animal using an imaging technique that provides single cell resolution of cells in the tissue, thereby generating a pharmacomap of the compound. In some aspects, provided herein is a method of generating a pharmacomap, comprising imaging a tissue of the non-human animal, wherein a compound has been administered to the animal, and wherein the imaging provides single cell resolution of cells in the tissue, thereby generating a pharmacomap of the compound. In certain embodiments, the non-human animal is sacrificed before the tissue is imaged. In other embodiments, the non-human is not sacrificed and the imaging technique is performed on a tissue of a live non-human animal. In specific embodiments, provided herein is a method of generating a pharmacomap, comprising; (a) administering a compound to a non-human animal; (b) harvesting a tissue of the animal; and (c) imaging the harvested tissue using an imaging technique that provides single cell resolution of cells in the tissue, thereby generating a pharmacomap of the compound. In some embodiments, the compound is a reference compound having a known therapeutic and/or toxicity effect. In certain embodiments, the non-human animal is a transgenic animal, for example, a non-human animal carrying a genetic regulatory region that controls expression of a detectable, e.g., fluorescent, reporter gene sequence. In some of the embodiments, the imaging technique used provides single cell resolution of cells expressing the reporter gene sequence in the tissue.

In one aspect, provided herein is method of generating a pharmacomap of a test compound for predicting therapeutic effects and/or toxicity effects of the test compound comprising: imaging a tissue using an imaging technique that provides single cell resolution of cells, wherein the tissue is from or in a non-human animal administered a test compound; identifying, by use of one or more data processors, cells that are activated in response to the test compound using a machine learning algorithm; generating a representation, by use of the one or more data processors, of the identified cells into a volume of continuous tissue space; performing, by use of the one or more data processors, statistical techniques to identify regions of significant differences based on a comparison of the generated representation of the identified cells of the harvested tissue and a representation of cells of a control tissue; and generating, by use of the one or more data processors, a pharmacomap of the test compound based on the identified regions of significant differences to identify anatomical tissue regions that are activated in response to the test compound for predicting therapeutic effects and/or toxicity effects of the test compound. In a specific embodiment, the non-human animal is a transgenic animal that includes a genetic regulatory region to control expression of a detectable, e.g., fluorescent, reporter gene sequence. In certain embodiments, the step of generating a representation of the identified cells into a volume of continuous tissue space comprises warping of the tissue images into a standard volume of continuous tissue space to register information associated with the identified cells within the continuous tissue space; and performing voxelization of the continuous tissue space to generate discrete digitization of the continuous tissue space. In some embodiments, the pharmacomap includes a representation of the continuous tissue space that includes one or more voxels, and includes pharmacomap information that identifies the activated anatomical tissue regions in the tissue space; wherein an activated anatomical tissue region comprises one or more voxels; and wherein a voxel includes one or more cells that are activated in response to the test compound. In certain embodiments, the step of generating a pharmacomap of the test compound includes performing an anatomical segmentation of the identified regions of significant differences. In some embodiments, the machine learning algorithm includes a convolutional neural network algorithm. In certain embodiments, the statistical techniques include a negative binomial regression technique, a random field theory technique, and/or one or more t-tests. In specific embodiments, the imaging technique includes a serial two-photon tomography. In some embodiments, the tissue is a whole organ, and the imaging technique described herein provides single cell resolution of cells in the whole organ (e.g., brain). In one embodiment, the methods described herein lead to generation of a pharmacomap of a whole organ (such as a brainwide pharmacomap).

In one embodiment, a method of generating a pharmacomap of a test compound is used for predicting therapeutic effects and/or toxicity effects of the test compound, comprising administering a test compound to a non-human animal; imaging a tissue using an imaging technique that provides single cell resolution of cells in the tissue; identifying, by use of one or more data processors, cells that are activated in response to the test compound using a machine learning algorithm; generating a representation, by use of the one or more data processors, of the identified cells into a volume of continuous tissue space; performing, by use of the one or more data processors, statistical techniques to identify regions of significant differences based on a comparison of the generated representation of the identified cells of the harvested tissue and a representation of cells of a control tissue; and generating, by use of the one or more data processors, a pharmacomap of the test compound based on the identified regions of significant differences to identify anatomical tissue regions that are activated in response to the test compound for predicting therapeutic effects and/or toxicity effects of the test compound. In another embodiment, a method of generating a pharmacomap of a test compound is used for predicting therapeutic effects and/or toxicity effects of the test compound, comprising administering a test compound to a non-human animal; harvesting a tissue of the animal; imaging the tissue using an imaging technique that provides single cell resolution of cells in the tissue; identifying, by use of one or more data processors, cells that are activated in response to the test compound using a machine learning algorithm; generating a representation, by use of the one or more data processors, of the identified cells into a volume of continuous tissue space; performing, by use of the one or more data processors, statistical techniques to identify regions of significant differences based on a comparison of the generated representation of the identified cells of the harvested tissue and a representation of cells of a control tissue; and generating, by use of the one or more data processors, a pharmacomap of the test compound based on the identified regions of significant differences to identify anatomical tissue regions that are activated in response to the test compound for predicting therapeutic effects and/or toxicity effects of the test compound. In some embodiments, the step of generating a representation of the identified cells into a volume of continuous tissue space comprises warping of the tissue images into a standard volume of continuous tissue space to register information associated with the identified cells within the continuous tissue space; and performing voxelization of the continuous tissue space to generate discrete digitization of the continuous tissue space. In some embodiments, the pharmacomap includes a representation of the continuous tissue space that includes one or more voxels, and includes pharmacomap information that identifies the activated anatomical tissue regions in the tissue space; wherein an activated anatomical tissue region comprises one or more voxels; and wherein a voxel includes one or more cells that are activated in response to the test compound. In some embodiments, the step of generating a pharmacomap of the test compound includes performing an anatomical segmentation of the identified regions of significant differences. In specific embodiments, the machine learning algorithm includes a convolutional neural network algorithm. In some embodiments, the statistical techniques include a negative binomial regression technique. In one embodiment, the statistical techniques include one or more t-tests. In one embodiment, the statistical techniques include a random field theory technique. In specific embodiments, the imaging technique includes a serial two-photon tomography. In some of these embodiments, the test compound is administered to a transgenic animal that carries a genetic regulatory region to control expression of a detectable, e.g., fluorescent, reporter gene sequence. In some of these embodiments, the imaging technique used provides single cell resolution of cells expressing the detectable, e.g., fluorescent, reporter gene sequence in the tissue.

In another aspect, described herein is a method for predicting the therapeutic effect and/or toxicity effect of a test compound comprising administering the test compound to a non-human animal, imaging a tissue of the animal using an imaging technique that provides single cell resolution, thereby generating a pharmacomap of the test compound, and comparing the pharmacomap of the test compound to that of the pharmacomap of a reference compound or to that of a database of pharmacomaps of reference compounds. In yet another aspect, described herein is a method for predicting the therapeutic effect and/or toxicity effect of a test compound comprising imaging a tissue of a non human animal, wherein the test compound has been administered to the animal, and wherein the imaging provides single cell resolution, thereby generating a pharmacomap of the test compound, and comparing the pharmacomap of the test compound to that of the pharmacomap of a reference compound or to that of a database of pharmacomaps of reference compounds. In certain embodiments, the non-human animal is sacrificed before the tissue is imaged. In other embodiments, the non-human is not sacrificed and the imaging technique is performed on a tissue of a live non-human animal. In a specific embodiment, described herein is a method for predicting the therapeutic effect and/or toxicity effect of a test compound comprising administering the test compound to a non-human animal, harvesting a tissue of the animal, imaging the harvested tissue using an imaging technique that provides single cell resolution, thereby generating a pharmacomap of the test compound, and comparing the pharmacomap of the test compound to that of the pharmacomap of a reference compound or to that of a database of pharmacomaps of reference compounds. In certain embodiments, the method of predicting therapeutic effects and/or toxicity effects of a test compound further comprises generating, by use of one or more data processors, a pharmacomap of the test compound by identifying anatomical tissue regions in the tissue (e.g., harvested tissue) that are activated in response to the test compound, wherein the pharmacomap includes a representation of a tissue space of the tissue (e.g., harvested tissue), and includes pharmacomap information that identifies the activated anatomical tissue regions in the tissue space. In some embodiments, the method further comprises comparing, by use of the one or more data processors, the pharmacomap of the test compound to a predetermined pharmacomap of a reference compound, wherein the reference compound has a known therapeutic or toxicity effect that correlates to the pharmacomap of the reference compound; and predicting the therapeutic effects or toxicity effects of the test compound based on the comparison of the pharmacomaps of the test compound and the reference compound. In certain embodiments, the step of predicting the therapeutic effects or toxicity effects of the test compound includes generating a correlation matrix of the reference compound between the known therapeutic or toxicity effect of the reference compound and the pharmacomap of the reference compound. In specific embodiments, the representation of the tissue space of the harvested tissue includes generation of a three-dimensional image of the harvested tissue, warping of the three-dimensional image into a standard volume of the tissue space, and voxelization of the tissue space to generate discrete digitization of the tissue space. In a specific embodiment, an activated anatomical tissue region comprises one or more voxels; and a voxel includes one or more cells that are activated in response to the test compound.

In certain embodiments, a machine learning algorithm is used to detect activated cells in the imaged tissue. In one embodiment, the machine learning algorithm is a convolutional neural network algorithm.

In certain embodiments, the methods described above further comprise warping of the imaged tissue (e.g. harvested tissue) into a volume of continuous tissue space; performing voxelization of the continuous tissue space to generate discrete digitization of the continuous tissue space; using statistical techniques upon the discrete digitization to identify areas of significant differences between control and drug-activated tissue areas; and using anatomical segmentation to assign the significant differences to tissue regions and to determine numbers of activated cells for one or more of the tissue regions, wherein the determined number of activated cells is used in comparing of the pharmacomap of the test compound to that of the pharmacomap of a reference compound.

In another aspect, described herein are methods for predicting therapeutic effects or toxicity effects of the test compound, wherein the test compound is administered to a non-human animal (e.g., a transgenic animal that includes a genetic regulatory region to control expression of a detectable, e.g., fluorescent, reporter gene sequence), wherein a tissue of the animal is harvested (or has been harvested), the method comprising: imaging the harvested tissue using an imaging technique that provides single cell resolution of cells (e.g., cells expressing the fluorescent reporter gene sequence) in the tissue; identifying, by use of one or more data processors, cells that are activated in response to the test compound using a machine learning algorithm; generating a representation, by use of the one or more data processors, of the identified cells into a volume of continuous tissue space; performing, by use of the one or more data processors, statistical techniques to identify regions of significant differences based on a comparison of the generated representation of the identified cells of the harvested tissue and a representation of cells of a control tissue; and generating, by use of the one or more data processors, a pharmacomap of the test compound based on the identified regions of significant differences to identify anatomical tissue regions that are activated in response to the test compound for predicting therapeutic effects or toxicity effects of the test compound. In some embodiments, the step of generating a representation of the identified cells into a volume of continuous tissue space comprises: warping of the tissue images into a standard volume of continuous tissue space to register information associated with the identified cells within the continuous tissue space; and performing voxelization of the continuous tissue space to generate discrete digitization of the continuous tissue space. In certain embodiments, the pharmacomap is stored in a computer-readable storage medium; wherein the computer-readable storage medium includes a storage area for storing voxel data that is representative of the continuous tissue space; wherein the computer-readable storage medium includes data fields for storing pharmacomap data that identifies the activated anatomical tissue regions in the tissue space represented by the voxel data; and wherein an activated anatomical tissue region comprises one or more voxels, and a voxel is representative of a tissue region having one or more cells that are activated in response to the test compound. In specific embodiments, the computer-readable storage medium is a database stored in a non-transitory storage medium, or a memory device. In some embodiments, the computer-readable storage medium includes pharmacomap data of one or more reference compounds which is associated with therapeutic effects or toxicity effects of the reference compounds upon particular regions of tissue; wherein the pharmacomap data of the test compound is compared with the pharmacomap data of the one or more of the reference compounds in order to predict the therapeutic effects or toxicity effects of the test compound. In certain embodiments, the step of generating a pharmacomap of the test compound includes performing an anatomical segmentation of the identified regions of significant differences. In specific embodiments, the machine learning algorithm includes one of the following: a convolutional neural network algorithm, support vector machines, random forest classifiers, and boosting classifiers. In particular embodiments, the statistical techniques include a negative binomial regression technique, one or more t-tests, and/or a random field theory technique. In some embodiments, the imaging technique includes one of the following: a serial two-photon tomography, Allen institute serial microscopy, all-optical histology, robotized wide-field fluorescence microscopy, light-sheet fluorescence microscopy, OCPI light-sheet, and micro-optical sectioning tomography.

In some embodiments, the non-human animal is a transgenic animal (e.g., a rodent such as a mouse or a rat). For example, a transgenic animal that carries a genetic regulatory region that controls expression of a detectable (e.g., fluorescent) reporter gene sequence can be used. In certain embodiments, imaging of the harvested tissue provides single cell resolution of cells expressing the detectable (e.g., fluorescent) reporter gene sequence in the tissue (such as cells activated by the test compound). In certain embodiments, the reference compound has a known therapeutic and/or toxicity effect. The reference compound can be one compound or two, three, four, or more than four compounds. In embodiments where the reference compound is more than one compound, the pharmacomap of the test compound can be compared to the “virtual” pharmacomap of reference compounds generated by averaging multiple reference compounds. The comparing of the pharmacomaps allows predicting the therapeutic effect or toxicity effect of the test compound based on the similarity of the pharmacomaps.

In certain embodiments, the tissue imaged in accordance with the methods described herein is brain, kidney, liver, pancreas, stomach, heart or any other tissue of a non-human animal. In specific embodiments, the tissue is a whole organ of a non-human animal (e.g., whole brain or whole liver). In some embodiments, the method comprises harvesting two or more than two tissues of a non-human animal (e.g., brain tissue and liver tissue). In some embodiments, the pharmacomap generated is that of an entire brain (e.g., of the transgenic animal).

In specific embodiments, the imaging technique used in the methods described herein is serial two-photon tomography, however, other imaging techniques (e.g., imaging techniques that provide single cell resolution of the imaged tissue) known in the art or described herein can also be used.

In some embodiments, the methods described herein are applied to a transgenic animal carrying a genetic regulatory region that controls expression of a detectable, e.g., fluorescent, reporter gene sequence. In certain embodiments, the genetic regulatory region is a genetic regulatory region of an immediate early gene (a gene that is rapidly and transiently activated in response to external stimuli in the absence of de novo protein synthesis, e.g., a gene that is activated within 10 minutes, within 20 minutes, or within 30 minutes, and that can be expressed within 1, 2, 3, 4 hours, or 6 hours of an activating stimulus). The genetic regulatory region can, for example, be a promoter or a region of a promoter. In specific embodiments, the immediate early gene is c-fos, FosB, delta FosB, c-jun, CREB, CREM, zif/268, tPA, Rheb, RGS2, CPG16, COX-2, Narp, BDNF, CPG15, Arcadlin, Homer-1a, CPG2, or Arc. In other embodiments, the genetic regulatory region is that of a late/secondary gene that is activated downstream of another gene (e.g., an immediate early gene) and that may require protein synthesis of the other gene (e.g., an immediate early gene). In some embodiments, the genetic regulatory region is that of a late/secondary gene that is activated more than 30 minutes, more than 1 hour, or more than 2 hours after a stimulus. In some embodiments, a late/secondary gene is expressed for more than 12 hours, more than 1, 2, 3, 4, 5 days, or more than 1, 2, 3, 4 weeks after a stimulus. In specific embodiments, the genetic regulatory region is that of neurofilament light chain, synapsins, glutamic acid decarboxylase (GAD), TGF-beta, NGF, PDGF, BFGF, tyrosine hydroxylase, fibronectin, plasminogen activator inhibitor-1, superoxide dismutase (SOD1), or choline acetyltransferase. In some embodiments, the reporter gene sequence encodes green fluorescent protein (GFP), although any marker that provides a detectable, e.g., fluorescent, signal known in the art or described herein can be used.

In a specific embodiment, the methods described herein are used for predicting therapeutic effect of the test compound, wherein the reference compound has a known therapeutic effect (e.g., in a human). In other embodiments, the methods described herein are used for predicting toxicity effect of the test compound, wherein the reference compound has a known toxicity effect (e.g., in a human). In another specific embodiment, the methods described herein are used for predicting an optimal dose of a test compound (e.g., a therapeutically effective dose and/or a dose that causes no or minimal toxicity or side effects). In some embodiments, the methods described herein are used for predicting an optimal dose of a test compound (e.g., a therapeutically effective dose and/or a dose that causes no or minimal toxicity or side effects), wherein the reference compound (which can be the same compound as the test compound at a different dose, or a different compound) has a known therapeutic effect or toxicity effect (e.g., in a human).

In some embodiments, the therapeutic effect of a test compound and/or reference compound is a therapeutic effect on a disorder or condition of the brain (e.g., central nervous system disorder). In some embodiments, the therapeutic effect of a test compound and/or reference compound is a therapeutic effect on a disorder or condition which is not a brain disorder or condition. In specific embodiments, the toxicity effect of a test compound and/or reference compound is a toxicity effect affecting brain function.

Any compound can be screened or analyzed using the described methodology. In some embodiments, the compound is a compound intended to be used in treating a disorder or condition (e.g., brain disorder). In other embodiments, the compound is a compound not intended to be used in treating a particular disorder or condition (e.g., a brain disorder or condition). In some of these embodiments, the compound is intended for use in treating any disease or condition which is not a brain disease or condition (e.g., cancer, heart disease, etc.), and a pharmacomap of the brain is generated as described herein. For example, such pharmacomap can be used to analyze whether the compound has or is predicted to have any brain-related side effects (e.g., central nervous system side effects).

Any compound(s) that is currently being used in the treatment of a disorder can be utilized as reference compound. In addition, any compound (s) that is not used in the treatment of a disorder (e.g., a compound that has failed in preclinical testing due to toxicity) can be utilized as a reference compound. In some embodiments, the reference compound is a drug used for treating a brain disorder. In other embodiments, the reference compound is a drug that is not used for treating a brain disorder. In particular embodiments, the reference compound is a drug that is not used for treating a brain disorder and has a known toxicity effect (e.g., known toxicity affecting brain function). In some embodiments, the test compound is a drug used for, or being considered for use in, treating a brain disorder. In certain embodiments, the test compound is predicted to have a therapeutic effect on a disorder or condition of the brain (e.g., central nervous system disorder). In other embodiments, the test compound is not predicted to have a therapeutic effect on a disorder or condition of the brain (e.g., central nervous system disorder). The methods described herein can be repeated with a plurality of test compounds. The pharmacomaps obtained for each of the test compounds can be compiled into a single database.

In some embodiments, the methods provided herein can be used for selection and/or design of new drugs based on the results of comparing of the pharmacomap of a test drug to the pharmacomap(s) of one or more reference drugs with known clinical outcomes (or to a database of pharmacomaps of reference drugs with known clinical outcomes).

### 4. BRIEF DESCRIPTION OF THE DRAWINGS

FIG. 1 illustrates operations for a pharmacomap data representation and analysis process.

FIG. 2 depicts a computer-implemented environment wherein users can interact with pharmacomap data representation and analysis systems hosted on one or more servers through a network.

FIG. 3 illustrates operations for generating pharmacomap data representations.

FIG. 4 illustrates different techniques that can be used to generate pharmacomap data representations.

FIG. 5 illustrates data that can comprise pharmacomap data.

FIG. 6 illustrates operations for analyzing test pharmacomaps with reference pharmacomaps for multiple purposes, such as to identify possible effects of the test compound.

FIG. 7 illustrates an implementation where the test pharmacomap information and the reference pharmacomap are stored in separate databases.

FIG. 8 illustrates an implementation where the test pharmacomap information and the reference pharmacomap are stored in the same database.

FIG. 9 illustrates an implementation where the test pharmacomap information has been generated and stored by a different company than the company which is to perform the test-reference pharmacomap analysis.

FIG. 10 illustrates an implementation where the test pharmacomap information has been generated and stored by the same company which is to perform the test and reference pharmacomap analysis.

FIG. 11. STP tomography. (a) Schema of the method. Computer-controlled XYZ stages moves the brain sample under the objective of a two-photon microscope, so that the top view is imaged as a mosaic. The stage also delivers the brain to a built-in vibrating blade microtome for sectioning. (b) 2D montage of a GFPM STP-tomography dataset comprising 260 coronal sections. (c) Coronal, horizontal and sagittal views of the GFPM dataset after 3D reconstruction. (d) A coronal section imaged with a 20× objective at 0.5 μm XY sampling. Left: 3D view of the coronal section and its position in the mouse brain (approximately −2.5 mm from Bregma). Panels 1 and 2: full views of the marked-up regions; scale bar=250 μm. Panels 1′ and 2′: enlarged views demonstrating visualization of dendritic spines (1′ and 1″) and fine axon fibers (2′); scale bar=25 μm (1′) and 5 μm (1″).

FIG. 12. Examples of different XY sampling resolutions for imaging dendritic spines. GFPM mouse brain was imaged with a 20× objective at (a) 0.5 μm and (b) 1 μm XY resolution or with a 10× objective at (c) 1 μm and (d) 2 μm XY resolution. The scale bar numbers are in microns. Note that row (a) (20×, 0.5 μm) is the same as shown in FIG. 11. The arrowheads in the left panels point to the regions magnified in the right panels.

FIG. 13. Examples of different XY sampling resolutions for imaging axons. Regions comprising only axons (marked by arrowheads) were selected in the same datasets as shown in FIG. 12. The scale bar numbers are in microns. The inverted grayscale images of axon fibers contain black bars indicating the cross-sections used to evaluate the resolution for imaging GFP-labeled axons in the plot profiles shown in the most right panels (the plot profiles were measure with ImageJ on tif 16 bit images with no digital zoom). Mean values (±SEM) from five plot profile measurements for each condition were (μm): 1.2±0.1 (a), 1.9±0.2 (b), 2.7±0.3 (c), and 3.9±0.3 (d) (note that the back aperture was more underfilled for the large 10× lens).

FIG. 14. Retrograde tracing by CTB-Alexa-488. (a) 3D view of a coronal section comprising the injection site (1) and several retrogradely labeled regions (2-4). Lower left: position of the section in the whole brain (approximately −1.15 mm from Bregma). (b) Coronal and sagittal views of the injection site. (c) Cortical regions marked up in (a), comprising: (1) the injection site in the barrel field of the primary somatosensory cortex (S1BF), (2) ipsilateral secondary somatosensory cortex (S2), (3) granular insular cortex (GI), and (4) contralateral S1BF. The panels (2-4) are shown with enlarged regions from supragranular and infragranular cortical layers comprising CTB labeled cells. The scale bar is 250 μm in panel (1) and 50 μm in the enlarged view of panel (2).

FIG. 15. Retrograde tracing by CTB-Alexa-488 (the same brain as in FIG. 14 is shown). (a) 3D views of selected coronal sections comprising the retrogradely labeled brain regions. (b) Brain areas marked up in (a) comprising: (1) ipsilateral and (2) contralateral ventolateral orbital cortex (VLO) (Bregma=+2.2 mm); (3) primary motor cortex (M1) (Bregma=+1.6 mm); (4) claustrum (Cla) and (5) M1 (Bregma=+1.4); (6) ectorhinal cortex (Ect), (7) secondary somatosensory cortex (S2), (8) barrel field primary somatosensory cortex (S1BF), (9) ventral posteromedial thalamus (VPM) and posterior thalamus (PO) (Bregma=−1.8 mm) Retrograde labeling of the contralateral VLO from S1BF has not been described before; see previous studies for comparison (Welker et al., Exp. Brain research. Exp. Hirnforschung 73:411-435 (1988); Aronoff et al., Eur. J. Neurosc. 31:2221-2233 (2010)). The scale bar is 250 μm in panel (1) and 50 μm in the enlarged view of panel (1). The Bregma estimates are based on comparison to the Mouse Brain Atlas by Paxinos and Franklin20.

FIG. 16. Anterograde tracing by AAV-GFP and brain warping. (a) 3D view of a coronal section comprising the injection site (1) and several anterogradely labeled regions (2-5). Lower left: position of the section in the whole brain (approximately −1.9 mm from Bregma). (b) Coronal and sagittal views of the injection site. (c) Brain regions marked up in (a), comprising: (1) the injection site (S1BF), (2) ipsilateral caudoputamen (CP), (3) axon fibers in the internal capsule (ic), (4) ventral posteromedial thalamus (VPM) and posterior thalamus (PO), and (5) contralateral barrel cortex (S1BF). The enlarged views show inverted grayscale images for better visualization of axon fibers and varicosities. The scale bar in (1) and the enlarged view of (2) is 250 μm. (d) One section from a combined “virtual” two-tracer dataset generated by warping AAV-GFP brain onto CTB-Alexa-488 brain. (e) Brain region marked up in (d) comprising motor cortex (M1) with overlapping anterograde (AAV-GFP) and retrograde (CTB-Alexa-488) labeling.

FIG. 17. Anterograde tracing by AAV-GFP (the same brain as in FIG. 16 is shown). (a) 3D views of selected coronal sections comprising anterogradely labeled brain regions. (b) Brain areas marked up in (a) comprising: (1) and (2) ventolateral orbital cortex (VLO) (Bregma=+3.2 and +2.1 mm, respectively); (3) motor cortex (M1) and (4) contralateral M1 (Bregma=1.1 mm); (5) barrel cortex (S1BF), (6) caudoputamen (CP), and contralateral (7) S1BF and (8) CP (Bregma=−1.4 mm); (9) perirhinal cortex (PRh), (10) ventral posteromedial thalamus (VPM) and posterior thalamus (PO), and (11) zona incerta (ZI) (Bregma=−2.5 mm); (12) anterior pretectal nucleus (APT) (Bregma=−3.1 mm); (13) superior colliculus (SC) and (14) pontine nucleus (PN) (Bregma=−4.1 mm); (15) PN (Bregma=−4.4 mm); and (16) spinal trigeminal nucleus (SP5) (Bregma=−5.8 mm) Anterograde labeling of contralateral motor cortex from S1BF has not been described before; see previous studies for comparison (Welker et al., 1988; Aronoff et al. 2010). The enlarged views show inverted grayscale images for better visualization of axon fibers and varicosities. The scale bar in both (1) and enlarged view of (2) is 250 μm. The Bregma estimates are based on the Mouse Brain Atlas by Paxinos and Franklin20.

FIG. 18. Evaluation of Z-plane consistency before and after sectioning. (a, a′) An optical plane imaged at Z-depth 90 μm below brain surface. (b, b′) An optical plane imaged at Z-depth 40 μm below brain surface after cutting a single 50 μm thick section. (c, c′) An overlay shows a close overlap of the two planes, demonstrating high consistency of the optical Z-plane before and after sectioning. Note the close overlap of labeled dendrites (long arrows). The scale are (a) 200 μm and (b) 100 μm. The image is taken from the SST-ires-Cre::Ai93 olfactory bulb.

FIG. 19. Quantification of warping accuracy. 42 landmark points of interest were manually selected in two different brains in the olfactory bulb, cortex, lateral ventricle, anterior commissure, lateral septum, fornix, hippocampus, optic track, amygadala, and cerebellum regions. The distance between each pair of corresponding points before and after warping is plotted. The mean (±SEM) of the displacement before and after warping was 749.5±52.1 and 102.5±45.0, respectively (line above: before warping; line below: after warping).

FIG. 20. Brain warping. Combined “virtual” two-tracer dataset generated by warping AAV-GFP brain onto CTB-Alexa-488 brain. Coronal, sagittal and horizontal views of the injection sites in the two brains. Motor cortex with overlapping anterograde (AAV-GFP, darker shade signal) and retrograde (CTB-Alexa-488, lighter shade signal) tracers from the two warped brains is shown in a selected 2D section. The overlap can be seen as a bright signal at the interface between the darker shade signal and the lighter shade signal, pinpointed by cross-lines.

FIG. 21. Computational detection of CTB-Alexa. Machine learning algorithms were trained to detect CTB-Alexa-488 labeling based on initial human markups and detect CTB-positive cells automatically. Example images of before (left) and after (right) prediction, and overlays (below).

FIG. 22. Whole-mount two-photon microscopy. The whole brain was imaged by automated mosaic imaging interleaved with vibratome-based tissue sectioning to remove the imaged regions.

FIG. 23. A test dataset. (A) Histone H2BGFP transgenic mouse brain with GFP labeling in all cells was imaged in 130 sections evenly spaced by 100 μm (x-y resolution 1 μm). (B) A coronal section with a single FOV enlarged from a mosaic of 9×13. (C) The sections re-aligned in 3D.

FIG. 24. Morphing. (A) An internal alignment between the brain generated in FIG. 23 and MRI brain atlas. Left: section imaged by the described method; middle: a morphed MRI section; right: an overlay of the two. (B) An example of anatomical segmentation from the MRI atlas. (C) Examples of anatomical segmentation of the test sample.

Example 25. c-fos-GFP labeling of activated brain regions. Strong labeling is induced in striatum (A) and lateral septum (B) in haloperidol-(A-B), but not saline-(C-D) treated c-fos-GFP mice. The brain was imaged as shown in FIG. 23. (scale bar=200 μm in A; 50 μm in the insert).

FIG. 26. Automated detection of c-fos-GFP. A) raw c-fos-GFP expression data (left) was analyzed by a convolutional neural network (middle) trained to detect c-fos-GFP from ground truth datasets marked by human observers. The output detection is shown on the right. B) enlarged view of input and output data showing a representative outcome of the current algorithm: out of 12 cells, 9 were identified correctly, one was missed (arrow on the left; false negative result) and two cells near each other were identified as one (arrow on the right; false negative result).

FIG. 27. Distribution of c-fos-GFP in the brains of mice injected with (A) saline or (B) haloperidol (1 mg/kg). (C) Preliminary quantitation of c-fos-GFP cells between the two samples per single coronal sections. The asterix marks the approximate position of c-fos-GFP expression in the striatum (B, C). Also, note in (C) the broad distribution of haloperidol-evoked c-fos-GFP induction in the caudal sections.

FIG. 28. Image voxelization. A-C: 19 different brains (A) are registered to one brain (B) to generate a reference brain (C) (average of 20 brains). D-F: Prediction results (F, centroids of c-fos-GFP cells) are registered to a reference brain (E) based on registration parameters from a sample (D) to a reference brain (E). (G) Diameter of each voxel is 100 μm and distance between each voxel is 20 μm. (H) Voxelized brain image.

FIG. 29. Schematic flowchart of the experimental design.

FIG. 30. Reconstruction of a series 2D sections. The imaged brain was reconstructed as a series of 2D sections, typically 280 to 300 per one mouse brain.

FIG. 31. Computational detection of c-fos-GFP. (A) convolutional neural networks learned inclusion and exclusion criteria of c-fos-GFP labeling based on human markups. (B) Examples of c-fos-GFP detection. Left, grayscale panels show raw data, right, black&white panels show computer-generated predictions, and below panels show an overlay.

FIG. 32. Raw data warping to a reference brain atlas. The serial 2D-section data set was reconstructed in 3D and warped onto a 3D reference brain volume generated as an average of twenty wild type brains scanned by STP tomography. The warping was done based on tissue autofluorescence, using elastix software.

FIG. 33. c-fos-GFP data registration to a 3D reference brain. Registration of c-fos-GFP data onto the reference brain creates a 3D representation of c-fos-GFP distribution, a c-fos-GFP pharmacomap. c-fos-GFP pharmacomaps of saline and haloperidol (1 mg/kg) brains with 176,771 and 545,838 c-fos-GFP cells, respectively.

FIG. 34. Voxelization of 3D c-fos-GFP data. The 3D brain volumes were voxelized as an evenly spaced grid of X-Y-Z=450×650×300 voxels, each voxel of size 20×20×50 microns, to generate discrete digitization of the continuous brain space. (A) heat-map distribution of c-fos-GFP in voxelized saline and haloperidol brains in 3D. (B) same brains in 2D montage.

FIG. 35. Statistical comparison. Heat maps of statistical differences between haloperidol (n=7) and saline (n=7) injected mice. Statistical comparison between the two groups was done by a series of negative binomial regressions. Type I error is corrected by setting a false discovery rate (FDR) of 0.01, under the assumption that the voxels have some level of positive correlation with each other.

FIG. 36. Social stimulation to investigate social brain circuitry. (A) Experimental design to examine c-fos-GFP changes after social exposure. (B) Three different groups for c-fos-GFP mice (N=7 mice per group).

FIG. 37. Serial two-photon tomography to examine entire brain with cellular resolution. (A) schematic picture of serial two-photon tomography, (B-D) montage view (D) of serial 2D reconstruction (C) after acquiring a series of individual image tiles (B). (E) 3D reconstruction of an entire brain.

FIG. 38. Machine learning algorithm for automatic detection of c-fos-GFP cells. (A) A computer learns inclusion and exclusion criteria of c-fos-GFP cells based on initial human markup and detects the positive cells automatically for new data set (prediction). (B-D) Example images of before (C) and after (D) prediction of a part of cortex (B).

FIG. 39. Image registration to a reference brain. (A-B) 19 different brains (A1 and A2) were registered to one brain (A) to generate a reference brain (B) (average of 20 brains). (C-E) Prediction results (E, centroids of c-fos-GFP cells) were registered to a reference brain (D) based on registration parameter from a sample (C) to a reference brain (D).

FIG. 40. Voxelization to measure c-fos-GFP cell increase. (A) Diameter of each voxel is 100 μm and distance between each voxel is 20 μm. (B-C) Each Voxelized brain image (B) was registered in the same space of the reference brain (C).

FIG. 41. Voxel-wise statistical analysis to identify brain areas responding to social exposure. (A-D) Averaged voxelization results registered to the reference brain (D) from handling control (A), object control (B), and social stimulation (C) group. (E) Montage shows brain areas activated after social exposure (C) compared to other two control groups (A and B). (F) 3D overlay of the activated brain area and the reference brain.

FIG. 42. Shared brain areas in autism mouse models fail to show significant c-fos increase after social stimulation. (A-B) summary of c-fos density in autism mouse models carrying neuroligin 4 KO (A) and neuroligin 3 R451C (B), *p<0.05. Underlines/bars under brain areas indicate brain areas which have significant c-fos increase in wild type littermates but not in Ngn 4 KO (A) and Ngn 3 R451C (B). (C) c-fos immunohistochemistry in neuroligin 4 wild type littermates showed significant increase in central amygdala and infralimbic cortex whereas neuroligin 4 KO didn't show similar increase after social exposure. scale bar=200 μm.

FIG. 43. 3D Image reconstruction. The entire brain was imaged in 8 blocks. Each block was scanned just as to encompass the brain region without the fixation medium. The blocks of different slices were aligned to a reference block using SIFT based method and entire brain was reconstructed in 3D.

FIG. 44. GAD-Cre detection and quantification. (A) Randomly selected 3D tiles from different regions of the brain were labeled by a human observer for the GAD-Cre signal. (B) This ground truth data was used to train a convolutional neural network for GAD-Cre signal detection. The training was done using a subset of images and then used on the rest of the brain image.

FIG. 45. Anatomical Segmentation. An MRI atlas was warped on to the brain image on the auto-fluorescence channel (resampled at 20 microns in x & y, 50 microns in z) using mutual information as constraint and thus using the same warping parameters; brain region labels were also warped. The resultant label was then resampled to original x, y, z resolutions and region wise counting was done.

FIG. 46 illustrates an example process for generating a pharmacomap of a drug.

FIG. 47 illustrates example pharmacomaps for haloperidol, risperidone, and aripiprazole, respectively.

FIG. 48 shows example pharmacomaps for different dosages of haloperidol.

FIG. 49 illustrates an example of generating a comprehensive database of pharmacomaps for predicting therapeutic and adverse effects of a new drug.

FIG. 50 illustrates example Principal Component Analysis (PCA) of adverse effects and indications for drugs.

FIG. 51 illustrates example representation of adverse effects for drugs.

FIG. 52 illustrates an example of data measuring similarity in pharmacomaps of haloperidol, risperidone, and aripiprazole.

### DETAILED DESCRIPTION

Provided herein, in one aspect, are high resolution, quantitative methods for analyzing reference compounds and for testing drug candidates in a non-human animal, e.g., an animal model. In one aspect, provided herein is technology for unbiased and quantitative mapping of drug-induced response in a tissue (e.g., whole brain) of a non-human animal at a single cell resolution. The method allows generation of a three-dimensional cellular activity pattern or a pharmacomap for each of the compounds tested. In another aspect, provided herein is technology for predicting the clinical effect of a test compound based on a computational analysis of similarities between the pharmacomap of the test compound and the pharmacomap(s) of one or more reference compounds that have known clinical effects. Correlation between new candidate drugs (such as test compounds) and drugs with known clinical effects (such as reference compounds) can be utilized to, for example, select the optimum candidates drugs that have the greatest chance to improve on existing therapeutics.

The non-human animal used in the methods described herein can be a rodent, e.g., a mouse or a rat. In some embodiments, the non-human animal is a transgenic animal, such as a non-human animal engineered to carry a foreign gene. In certain embodiments, the non-human animal used in the methods described herein has been engineered to carry a detectable, e.g., fluorescent, reporter gene sequence under the control of a genetic regulatory region. In specific embodiments, drug-induced stimulation of cells of the analyzed tissue results in transcriptional activation of the genetic regulatory region leading to protein expression of the reporter gene. In some of these embodiments, the genetic regulatory region is a genetic regulatory region, e.g., a promoter, of an immediate early gene (IEG), such as a gene that is rapidly activated and expressed in response to external stimuli in the absence of de novo protein synthesis (e.g., mRNA of IEG can be produced within minutes such as within 5, 10, 20, 30, 40, 50 or 60 minutes, and a protein can be expressed within 30 or 45 minutes, or 1, 2, 3, 4, 5, or 6 hours after drug administration). In other embodiments, the genetic regulatory region is a genetic regulatory region, e.g., promoter, of a late gene, such as a gene that is activated downstream of immediate early gene activation, or that is activated more than 30 minutes after a stimulus (such gene can be expressed for more than 12 hours, more than 1, 3, 5 days, or 1, 2, 3, 4 weeks, after drug administration). In such embodiments, the expression of a reporter gene provides a read-out for drug induced cellular activation.

In other embodiments, drug-induced expression and/or activity of a native, endogenous gene is analyzed in a tissue of a non-human animal. In some of these embodiments, the non-human animal is not a transgenic animal. In these embodiments, analysis of drug-induced pattern of cellular activity is performed using techniques known in the art, such as immunohistochemistry or in situ hybridization.

In certain embodiments, the non-human animal used in the methods described herein is an animal of a wild-type phenotype (e.g., not carrying a mutation associated with a diseases state). In other embodiments, the non-human animal used in the methods described herein is an animal of a mutant phenotype (e.g., carrying a mutation associated with a diseases state). For example, a non-human animal that can be used as described herein can be an animal model for a disease or condition of the brain, an animal model for any type of cancer, or an animal model for a heart condition, diabetes or stroke. In some embodiments, a non-human animal of a wild-type phenotype or a non-human animal of a mutant phenotype is engineered to carry a detectable, e.g., fluorescent, reporter gene sequence under the control of a genetic regulatory region for use in the methods described herein. In other embodiments, a non-human animal of a wild-type phenotype or a non-human animal of a mutant phenotype used in the methods described herein does not carry a detectable, e.g., fluorescent, reporter gene sequence under the control of a genetic regulatory region.

In some embodiments, the non-human animal used in the methods described herein is subjected to behavioral conditioning (e.g., fear conditioning or the “learned helplessness” conditioning), such as behavioral conditioning known or expected to result in a state similar to a disease state (e.g., a disease of the brain such as psychosis or depression). In some embodiments, the methods described herein can be used to predict a therapeutic (against a disease state) or toxicity effect of a drug in a non-human animal that has been subjected to behavioral conditioning known or expected to induce the disease state or a state similar to the disease state. For example, the methods described herein can be used to test or screen anxyolitic(s) in a non-human animal subjected to fear conditioning, or to test or screen antidepressant(s) in a non-human animal subjected to the “learned helplessness” conditioning.

In specific embodiments, a drug is administered to a group of non-human animals, wherein a certain number of the animals in the group are sacrificed and analyzed in accordance with the methods described herein (e.g., imaged to generate a pharmacomap), and wherein a certain number of the animals in the group are not sacrificed and instead their behavior is assessed and/or monitored using any methodology described herein or known in the art. In such embodiments, pharmacomaps generated in accordance with the methods described herein can be compared to or correlated with the behavioral responses to the drug in non-human animals.

In certain embodiments, a compound (e.g., a test compound or a reference compound) is administered to a non-human animal (e.g., a transgenic animal), and the animal is sacrificed by any method described herein or known in the art within a certain time period after drug administration (e.g., within 1 hour, 2 hours, 3 hours, 4 hours, 6 hours, 8 hours, 10 hours, 12 hours, 18 hours, 24 hours, 2 days, 3 days, 5 days, 1 week, 2 weeks, 1 month, or 2 months after drug administration). Subsequently, one or more tissues of the sacrificed animal can be harvested by any technique described herein or known in the art. In specific embodiments, the tissue is an entire organ of an animal (e.g., a brain and/or a liver). The harvested tissue can be analyzed (e.g., imaged) using any technique described herein or known in the art. In a specific embodiment, the imaging technique used provides very high (e.g., single cell) resolution of the cells of the harvested tissue (e.g., an entire organ).

In other embodiments, a non-human animal is not sacrificed after compound administration, and a tissue or tissues (e.g., a whole organ) of a live animal are analyzed (e.g., imaged) using any technique described herein or known in the art. In certain embodiments, after administration of a compound (e.g., a reference or test compound) to a non-human animal, a tissue or tissues from the animal are harvested and imaged using any technique described herein or known in the art, but the animal is not sacrificed. In some of these embodiments, the imaging technique used provides very high (e.g., single cell) resolution of the cells of the analyzed tissue.

In yet other embodiments, a non-human animal is sacrificed after a compound administration but a tissue is not harvested for analysis (e.g., imaging).

In some embodiments, a tissue of non-human animal that has not been treated with a drug (e.g., a test drug or a reference drug) is analyzed (e.g., imaged) using any technique described herein or known in the art. The tissue to be analyzed (e.g., imaged) can be harvested from a sacrificed non-human animal. Alternatively, the tissue to be analyzed can be harvested from a live animal. In other embodiments, the tissue is analyzed (e.g., imaged) in a live animal.

Automated microscopy (e.g., serial two-photon (STP) tomography) can be used for high-resolution imaging of a tissue of an animal treated with a test drug or a reference drug (e.g., a transgenic animal engineered to express a detectable, e.g., fluorescent, reporter gene in response to a stimulus). In certain embodiments, automated microscopy can be combined with image processing and computational methods for analysis of the acquired datasets. The methodology used provides high-resolution information regarding distribution pattern of activated cells in a three-dimensional space of the imaged tissue, thereby generating a pharmacomap of the tested compound. In a specific embodiment, the pharmacomap represents the number of activated cells expressing a detectable, e.g., fluorescent, reporter gene in specific regions of the imaged tissue in response to a stimulus (such as administration of a drug, e.g., a reference compound or a test compound). In certain embodiments, the resolution achieved is a single cell resolution. In some embodiments, the resolution achieved is 1 micron x-y resolution. In specific embodiments, the resolution achieved is between about 0.2 microns and about 20 microns, between about 0.2 microns and about 15 microns, between about 0.25 microns and 15 microns, between about 0.25 microns and about 10 microns, between about 0.25 microns and about 7.5 microns, between about 0.25 microns and about 5 microns, between about 0.25 microns and about 3 microns, between about 0.25 microns and about 2 microns, between about 0.25 microns and about 1 micron, between about 0.3 microns and about 15 microns, between about 0.3 microns and about 10 microns, between about 0.3 microns and about 5 microns, between about 0.3 microns and about 3 microns, between about 0.3 microns and about 1 micron, between about 0.4 microns and about 15 microns, between about 0.4 microns and about 10 microns, between about 0.4 microns and about 7.5 microns, between about 0.4 microns and about 5 microns, between about 0.4 microns and about 3 microns, between about 0.4 microns and about 2 microns, between about 0.4 microns and about 1 micron, between about 0.5 microns and about 15 microns, between about 0.5 microns and about 10 microns, between about 0.5 microns and about 7.5 microns, between about 0.5 microns and about 5 microns, between about 0.5 microns and about 3 microns, between about 0.5 microns and about 2 microns, or between about 0.5 microns and about 1 micron x-y resolution. In some embodiments, the highest resolution achieved is 0.2, 0.25, 0.3, 0.4 or 0.5 micron x-y resolution. In some embodiments, the lowest resolution achieved is 20, 15, 12.5, 12, 11, 10, 9, 8, 7, 6, 5, 4, 3, 2, 1.5, 1.25, 1, 0.75, or 0.5 micron x-y resolution.

In some embodiments, the imaged tissue is an entire organ, e.g., brain, heart, liver or any other organ of a non-human animal. Application of this method to a whole organ (e.g., whole brain) allows construction of detailed dose-response pharmacomaps of drug-induced organ-wide cellular activation at a single cell resolution (as measured, e.g., by expression of a detectable, e.g., fluorescent, reporter gene regulated by an immediate early gene promoter). In other embodiments, the imaged tissue is a piece, part or section of an organ.

Further, statistical methods can be used to compare the activation pattern of a tissue/pharmacomap produced by the test compound with the activation pattern of a tissue/pharmacomap produced by a reference compound, where the reference compound has a known therapeutic or toxicity effect (e.g., in a human). This methodology allows prediction of the therapeutic effect and/or toxicity effect of a test compound based on similarities and/or differences of the pharmacomaps of the test compound and one or more reference compounds. In specific embodiments, the reference compound(s) are structurally or functionally similar to the test compounds such that they are expected to activate similar regions of an organ or tissue imaged.

In specific embodiments, the imaging technique used in the methods described herein is STP tomography (for general description of the technology see U.S. Pat. No. 7,724,937 or Ragan et al., Nature Methods 9(3):255-258 (2012), each of which is incorporated by reference herein in its entirety). STP tomography integrates fast two-photon imaging and vibratome-based sectioning of a fixed tissue. Using this method, first the entire top view of a tissue can be imaged as a mosaic of individual field of views; then, the tissue can be moved towards a built-in vibratome that cuts off the imaged section; next, the tissue can be moved back under the microscope and the cycles of mosaic imaging and sectioning can be repeated until the entire tissue is imaged.

In certain embodiments, the fixed tissue or organ (e.g., whole brain) is embedded, e.g., in agar, for imaging using a high-throughput imaging technique such as STP tomography. Embedding the tissue in agar is advantageous because it results in maximal preservation of the fluorescent signal from a fluorescent reporter gene. In some embodiments, the agar-embedded organ or tissue is cross-linked prior to imaging (e.g., covalently cross-linked). In one embodiment, the surface of the tissue or organ (e.g., whole brain) is covalently cross-linked to agarose. Cross-linking of the tissue-agar interface allows to keep the tissue firmly embedded during sectioning of the imaged tissue. In certain embodiments, whole-mount microscopy is contemplated herein, where an entire organ or tissue (i.e, the whole brain) can be automatically imaged using STP tomography.

In certain embodiments, the methods described herein achieve the whole-mount mode of imaging of a tissue, high speed of imaging, and complete automation of data collection. Whole-mount imaging allows imaging of an intact top of a tissue or organ (e.g., a brain) before mechanical sectioning of the imaged region, which eliminates all tissue damage and distortion artifacts that occur during handling of cut brain sections in traditional serial microscopy. Further, in some embodiments, the methods described herein achieve rapid (1.4 kHz) collection of the large amount of data (e.g., 100 GB per one mouse brain) (using, for example, STP tomography). Further, in some embodiments, the methods contemplated herein allow complete automation of imaging and sectioning, transforming labor intensive serial microscopy of mouse brain sections into a high-throughput method that can be readily scaled up. In some of these embodiments, the imaging technique used is STP tomography.

In another aspect, provided herein is automated computational processing and analysis of the data obtained by the described imaging techniques providing a quantitative read-out. In some aspects, the described methods provide an integrated set of software, including automated detection of the activated detectable, e.g., fluorescent, reporter-positive cells by machine learning algorithms, warping of the imaged tissue onto one standard tissue volume, voxelization of the volume of the tissue to generate discrete digitization of the continuous tissue space, the use of statistics to identify areas of significant differences between control and drug-activated tissues, and the use of anatomical segmentation to assign these differences to specific regions of the tissue and to express the data as numbers of activated cells per anatomical structures and regions of the tissue.

The described methodology for imaging and image processing is fast, sensitive, cheap and has a minimal labor requirement. The generated pharmacomap measurements enable detailed comparisons of cellular activation in a non-human animal in response to, e.g., related drugs, such as chemically engineered versions of the same drug aimed at improving efficacy or limiting side-effects.

The described methods can also be used for screening of drugs that are or have been used in the clinics and have a known clinical outcome (e.g., in a human). Such screening can be used for construction of a reference pharmacomap database. For example a large scale pharmacomap database of reference drugs with known therapeutic and/or toxicity effects can be constructed (e.g., a database comprising more than 10, 20, 30, 40, 50, 60, 70, 80, 90, 100, 120, 150, 200, 250, 300, 500, 750, or more than 1000 pharmacomaps of drugs with a known clinical outcome). In specific embodiments, the clinical outcome is a therapeutic effect or a toxicity effect. In some embodiments, further generation of a computational correlation matrix linking the pharmacomaps of reference drugs and the clinical effects of the reference drugs is contemplated herein. Such pharmacomap databases can be used to provide predictive comparison between effects of drugs in a non-human animal and clinical effects of drugs (e.g., in humans).

In certain embodiments, the methods described herein can be used to determine an optimal dose of a drug for administration to a subject (e.g., a dose that provides an optimal therapeutic effect and/or minimal toxicity effect when administered to a subject). In some embodiments, the methods described herein can be used for screening a drug at two, three or more dosages (e.g., predicting the therapeutic effects and/or toxicity effects of two, three or more dosages of a test drug), and selecting the dosage that is predicted to achieve a therapeutic effect and/or predicted to cause minimal or no toxicity (e.g., minimal or no serious side effects). In some embodiments, a reference pharmacomap database generated using the methods described herein comprises pharmacomaps of a reference drug administered at two, three or more dosages (such as a medium dosage, a low dosage, and/or a high dosage; or a therapeutically effective dosage, a dosage that is not therapeutically effective, and/or a dosage that is known to cause one or more side effects).

In particular embodiments, the pharmacomaps described herein can be combined with information about structural, physical, and chemical properties (SPCPs) of the tested compounds. In other specific embodiments, the pharmacomaps described herein can be combined with any available information about properties (e.g., side effects) of the tested compounds. For example, the pharmacomaps described herein can be combined with information about properties of the tested compounds available through a database such as Pubchem, BioAssays or ChemBank (which, e.g., may contain information about drug-target interactions and/or cellular phenotypes induced by the drug(s)). In one embodiment, the pharmacomaps described herein can be combined with information about side effects of the tested compounds, e.g., information available through a database such as SIDER. In a particular embodiment, the pharmacomaps described herein can be combined with the data from the SIDER database.

### Screening of Drugs Affecting Brain Functions

In a particular aspect, provided herein is a drug-screening approach that can reliably predict therapeutic and/or toxicity outcomes of drugs affecting brain functions in a patient (e.g., a human). In such embodiments, cellular activity in the non-human animal brain in response to drug administration is analyzed. For example, a drug that affects brain function can be administered to a non-human animal (e.g., a mouse); the brain tissue (e.g., a whole brain) can be harvested by any technique known in the art and imaged at high resolution yielding a pharmacomap of the drug. Generation of detailed maps of drug-activated neurons (e.g., in the whole mouse brain) can be used to reliably link drug-evoked brain activation in a non-human animal model and drug-evoked clinical effects in humans. One of the drug-screening approaches provided herein comprises: 1) generation of a database of animal brain pharmacomaps for drugs with known human outcomes (“reference drugs” or “reference compounds”), 2) generation of a computational correlation matrix linking the reference animal brain pharmacomaps and the human effects of the reference drugs, and 3) the use of this correlation matrix to predict therapeutic effects of new test drugs (or new combinations of reference drugs) by comparing their pharmacomaps to the reference pharmacomap database.

In specific embodiments, the above-described drug screening can be achieved by ex-vivo imaging of brains of transgenic animals expressing a detectable, e.g., fluorescent, reporter gene (e.g., GFP) under the control of the activity-regulated promoter of the immediate early gene (IEG) (e.g., c-fos or Arc). In other specific embodiments, this can be achieved by ex-vivo imaging of brains of transgenic animals expressing a detectable, e.g., fluorescent, reporter gene (e.g., GFP) under the control of the activity-regulated promoter of a late gene. A late gene can be any gene that is activated downstream of and requires protein synthesis of another gene (e.g., an immediate early gene), or that is activated via other slow (more than 30 minutes) cellular signaling mechanism. An automated high-throughput imaging technique (e.g., that allows imaging of the entire brain) can be used to image the brain tissue of such transgenic animals (which express the detectable, e.g., fluorescent, reporter gene as a cellular marker of IEG expression in neurons that are activated by the screened drug). In one embodiment, the technique is STP tomography. Next, computational analysis of the detectable, e.g., fluorescent, reporter gene expression in the brain tissue can be performed using machine learning algorithms. Then, 3D animal model-brain pharmacomaps can be generated, wherein such pharmacomaps represent the number of activated neurons expressing the reporter gene in specific brain regions in response to the screened drug. In some embodiments, the imaging technique used in the methods described herein provides cellular brainwide resolution (e.g., at a throughput of one entire brain dataset per day). In some embodiments, the pharmacomaps of screened drugs obtained using the methods described herein comprise exact numbers and/or locations of cells expressing a detectable reporter gene in the whole brain of a non-human animal (such as drug-activated cells).

Using the above-described methodology, pharmacomaps of reference drugs with known clinical outcomes (e.g., in a human) can be compiled to create a reference database. The reference drugs can be any drugs that are or have been used for treating brain disorders, as well as drugs that failed in clinical trials as long as there is available information about the clinical effects of the drug (e.g., in a human). Then, transgenic animal brain pharmacomaps and known clinical effects of each drug can be plotted in the same matrix, creating correlations between neural activation in the mouse brain and clinical outcomes (e.g., in a human) In certain embodiments, if N different drugs (e.g., 5, 6, 7, 8, 9, 10, or more than 5, 6, 7, 8, 9, 10) showed overlapping activation in mouse brain regions X and Y and were known to cause a common therapeutic effect, it would be predicted that simultaneous X and Y activation in the mouse brain represents the common human outcome of these drugs. Similarly, if N drugs (e.g., 2, 3, 4, 5, 6, 7, 8, 9, or 10) shared a therapeutic effect not seen by the other n drugs (e.g., 3, 4, 5, 5, 7, 8, 9, 10, or more than 3, 4, 5, 6, 7, 8, 9, 10) and showed activation in an additional brain region Z, it would be assumed that the mouse brain region Z represents the selective effect of the N drugs. Any of the drugs that are currently being used in the treatment of brain disorders can be utilized to create the reference database. Further, any drugs that are not used in the treatment of brain disorders (e.g., those that failed preclinical testing) can be utilized to create the reference database (e.g., drugs that have known clinical effects such as toxicity effects). Subsequently, the mouse brain pharmacomap pattern of a test drug can be compared to the reference database, and the overlap of activation patterns of the template drugs can be used to predict the possible therapeutic effect and/or toxicity effect of the test drug. This method can be used for new drugs, as well as new combinations of drugs already used in the clinics.

Any compound can be screened or analyzed using the described methodology. In some embodiments, the compound is a compound intended to be used in treating a brain disorder or condition. In other embodiments, the compound is a compound not intended to be used in treating a brain disorder or condition. In some of these embodiments, the compound is intended for use in treating any disease or condition which is not a brain disease or condition (e.g., cancer, heart disease, etc.), and a pharmacomap of the brain is generated as described herein. For example, such pharmacomap can be used to analyze whether the compound has or is predicted to have any brain-related side effects (e.g., CNS side effects).

The above-described methodology for screening drugs affecting brain functions can also be applied to screening drugs that affect functioning of any other tissue or organ of a patient.

### 5.1 Transgenic Animals

The transgenic animals used in accordance with the methods provided herein are non-human animals in which one or more of the cells of the animal comprises a transgene.

### 5.1.1 Transgene

The transgenic animals used in the methods provided herein comprise a transgene(s) that comprises one or more genetic regulatory regions that are capable of controlling the expression of a reporter gene sequence such as a detectable, e.g., fluorescent, reporter gene. In certain embodiments, the genetic regulatory region is a genetic regulatory region of an immediate early gene, i.e., a gene that is activated transiently and rapidly in response to a stimulus, e.g., in response to a reference drug. In certain embodiments, the genetic regulatory region is a genetic regulatory region of a late/secondary gene, e.g., a gene that is activated downstream of another gene and that may require protein synthesis of another gene (e.g., an immediate early gene), or a gene that is activated via another slow cellular signaling mechanism (e.g., activated more than 30 minutes, more than 45 minutes, more than 1 hour, more than 3 hours, or more than 6 hours after a stimulus). A late/secondary gene can be expressed within 1, 2, 3, 4, 6, 8, 10, 12, or 24 hours of a stimulus. A late/secondary gene can be expressed for more than 12 hours, 1 day, 1 week, 2 weeks, 3 weeks, or 4 weeks after a stimulus).

In one aspect, the transgenic animals used in the methods provided herein comprise a transgene that comprises the genetic regulatory region of one or more immediate early genes. In certain embodiments, the genetic regulatory region may be from an immediate early gene that is activated immediately after a stimulus. In certain embodiments, the genetic regulatory region may be from an immediate early gene that is activated about 10 seconds, 20 seconds, 30 seconds, 40 seconds, 50 seconds, or one minute after a stimulus. In certain embodiments, the genetic regulatory region may be from an immediate early gene that is activated within 2 minutes, 3 minutes, 4 minutes, 5 minutes, 10 minutes, 15 minutes, 20 minutes, 25 minutes, 30 minutes, 45 minutes, or 1 hour after a stimulus. In certain embodiments, an immediate early gene is activated directly by a stimulus and does not require protein synthesis of another gene. In certain embodiments, the genetic regulatory region may be from an immediate early gene that is activated about 0 seconds to about 10 seconds, about 1 second to about 10 seconds, about 10 seconds to about 20 seconds, about 30 seconds to about 40 seconds, about 50 seconds to about 1 minute, or about 1 second to about 1 minute, after a stimulus. In certain embodiments, the genetic regulatory region may be from an immediate early gene that is activated about 1 minute to about 2 minutes, about 1 minute to about 5 minutes, about 5 minutes to about 10 minutes, about 10 minutes to about 20 minutes, about 20 minutes to about 30 minutes, about 1 minute to about 30 minutes, about 1 second to about 30 minutes, or about 1 second to about 45 minutes after a stimulus.

In certain embodiments, the genetic regulatory region may be from a gene that is activated about 30 minutes to about 1 hour, about 1 hour to about 1.5 hours, about 1 hour to 2 hours, about 2 hours to 3 hours, or about 3 hours to about 4 hours after a stimulus. In certain embodiments, the genetic regulatory region may be from a gene that is activated about 45 minutes, about 1 hour, about 1.5 hours, 2 hours, 2.5 hours, 3 hours, 3.5 hours, or 4 hours after a stimulus.

The terms “about” and “approximately,” when used herein to a modify numeric value or numeric range, indicate that reasonable deviations from the value or range, typically 10% above and 10% below the value or range, remain within the intended meaning of the recited value or range.

Exemplary immediate early genes from which the genetic regulatory regions could be utilized include, without limitation, the genes that encode CREB, c-fos, FosB, delta FosB, c-jun, CREM, zif/268, tPA, Rheb, RGS2, CPG16, COX-2, Narp, BDNF, CPG15, Arcadlin, Homer-1a, CPG2, and Arc. Such genetic regulatory regions are well-known to one skilled in the art. In a specific embodiment, the immediate early gene used in accordance with the methods described herein is c-fos. Those skilled in the art will recognize that the genetic regulatory regions from other immediate early genes currently known or later discovered could be utilized in accordance with the methods described herein. In some embodiments, the genetic regulatory region is the genetic regulatory region of a human immediate early gene.

In another aspect, the transgenic animals used in the methods provided herein comprise the genetic regulatory region of one or more late/secondary genes, i.e., a gene that is not an immediate early gene. In some embodiments, a late/secondary gene is a gene that is activated downstream of another gene such as an immediate early gene (and, e.g., requires protein synthesis of another gene such as an immediate early gene). In some embodiments, a late/secondary gene is a gene that is activated via another slow cellular signaling mechanism (e.g., activated more than 30 minutes, more than 45 minutes, more than 1 hour, more than 2 hours, more than 4 hours, more than 6 hours, or more than 12 hours after a stimulus). In certain embodiments, the genetic regulatory region may be from a late/secondary gene that is activated within 45 minutes, 1 hour, 2 hours, 3 hours, 4 hours, 6 hours, 8 hours, 10 hours, 12 hours, or 24 hours after a stimulus. In certain embodiments, the genetic regulatory region may be from a late/secondary gene that is expressed about 1 hour, 2 hours, 3 hours, 4 hours, 4.5 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or 1 day after a stimulus. In certain embodiments, the genetic regulatory region may be from a late/secondary gene that is expressed for about 2 days, 3 days, 4 days, 5 days, 6 days, or 1 week after a stimulus. In certain embodiments, the genetic regulatory region may be from a late/secondary gene that is expressed for about 2 weeks, 3 weeks, 4 weeks, 1 month, or greater than 1 month after a stimulus. In certain embodiments, the genetic regulatory region may be from a late/secondary gene that is expressed about 1 hour to about 4 hours, 4 hours to about 6 hours, about 6 hours to about 12 hours, about 12 hours to about 1 day, about 1 day to about 2 days, about 3 days to about 5 days, about 5 days to about 1 week, about 1 week to about 2 weeks, about 2 weeks to about 3 weeks, or about 3 weeks to about 1 month after a stimulus.

Exemplary late/secondary genes from which the genetic regulatory regions could be utilized include, without limitation, the genes that encode neurofilament light chain, synapsins, glutamic acid decarboxylase (GAD), TGF-beta, NGF, PDGF, BFGF, tyrosine hydroxylase, fibronectin, plasminogen activator inhibitor-1, superoxide dismutase (SOD1), and choline acetyltransferase. Such genetic regulatory regions are well-known to one skilled in the art. Those skilled in the art will recognize that the genetic regulatory regions from other late/secondary genes currently known or later discovered could be utilized in accordance with the methods described herein. In some embodiments, the genetic regulatory region is the genetic regulatory region of a human late/secondary gene.

In some embodiments, the genetic regulatory region of an immediate early gene and a late/secondary gene is activated in a specific tissue or tissues (e.g., brain, liver, heart, or any other tissue.). See Loebnch & Nedivi, Physiol. Rev. 89:1079-1103 (2009); Clayton, Neurobiology, Learning and Memory 74:185-216 (2000).

In another aspect, the transgenic animals used in the methods provided herein comprise a transgene that comprises the genetic regulatory region of an immediate early gene and a late/secondary gene.

In certain embodiments, a transgene comprises the complete promoter of the gene.

In certain embodiments, a transgene comprises the complete promoter of a gene as well as additional nucleic acids of the gene. For example, the genetic regulatory region comprises the promoter of a gene of interest and additionally comprises about or at least 10, 20, 30, 40, 50, 60, 70, 80, 90, 100, 150, 200, 300, 400, 500, 1000, 2000, 3000, 4000, or 5000 nucleic acids of the gene.

In certain embodiments, a transgene comprises the complete promoter of a gene as well as additional nucleic acids of the gene and/or of neighboring DNA sequences (e.g., DNA sequences, introns or exons that are either upstream or downstream of the gene as it appears in its natural state (e.g., in the body of a subject) or as it appears in an engineered DNA construct (e.g., a plasmid or an amplified piece of DNA). For example, the genetic regulatory region comprises the promoter of a gene of interest and additionally comprises about or at least 10, 20, 30, 40, 50, 60, 70, 80, 90, 100, 150, 200, 300, 400, 500, 1000, 2000, 3000, 4000, or 5000 nucleic acids of the gene and/or of neighboring DNA sequences.

In certain embodiments, a transgene comprises a promoter of a gene as well as tens to hundreds of kilobases of additional nucleic acids. In a specific embodiment, such a genetic regulatory region is generated as (or as part of) a bacterial artificial chromosome (BAC) or as (or as part of) a yeast artificial chromosome (YAC).

In some embodiments, a transgene comprises a fragment of the genetic regulatory region of a gene such as a promoter (e.g., a fragment of a native gene promoter). In specific embodiments, the fragment of the genetic regulatory region is effective to facilitate transcription of the gene. In some embodiments, the fragment constitutes more than 20%, 30%, 40%, 50%, 60%, 70%, 75%, 80%, 90%, 95%, 98%, of 99% of the genetic regulatory region of a gene (e.g., a native promoter). In some embodiments, the genetic regulatory region of a gene used in the methods described herein is a native genetic regulatory region that has been mutated (e.g., one or more nucleotides of the genetic regulatory region have been deleted or substituted, or one or more nucleotides have been added to the native regulatory region).

In certain embodiments, a transgene comprises a native gene promoter of the transgenic animal (e.g., transgenic mouse), wherein the native gene promoter is linked to a reporter gene. Methods of generating such transgenic mice are known in the art and described herein (see, e.g., Section 5.1.3).

### 5.1.2 Detectable Reporter Genes

Any reporter gene known to those of skill in the art may be used in the genetic regulatory region-reporter gene constructs described herein. Reporter genes refer to a nucleotide sequence encoding a protein that is readily detectable either by its presence or activity. In specific embodiments, a reporter gene comprises the coding region of a gene (e.g., a gene sequence that does not comprise intron sequence). Reporter genes may be obtained and the nucleotide sequence of the reporter gene determined by any method well-known to one of skill in the art.

In a specific embodiment, the reporter gene is a fluorescent reporter gene. Examples of fluorescent reporter genes include, but are not limited to, nucleotide sequences encoding green fluorescent protein (“GFP”) and derivatives thereof (e.g., fluorescent protein, red fluorescent protein, cyan fluorescent protein, and blue fluorescent protein), luciferase (e.g., firefly luciferase, renilla luciferase, genetically modified luciferase, and click beetle luciferase), and coral-derived cyan and red fluorescent proteins (as well as variants of the red fluorescent protein derived from coral, such as the yellow, orange, and far-red variants). In a specific embodiment, nucleotide sequences encoding GFP is derived from jellyfish Aequorea (e.g., Aequorea Victoria), or a coral (e.g., Renialla reniforms, Galaxeidae). In some embodiments, nucleotide sequences encoding cyan fluorescent protein is derived from a reef coral (e.g., Anemonia majano, Clavularia or Acropara). In some embodiments, nucleotide sequences encoding red fluorescent protein is derived from a coral (e.g., Discosoma, Heteractis crispa).

In another specific embodiment, the detectable reporter gene is not a fluorescent reporter gene, e.g., the reporter gene is a catalytic reporter gene. Examples of catalytic reporter genes include, without limitation, beta-galactosidase (“β-gal”), beta-glucoronidase, beta-lactamase, chloramphenicol acetyltransferase (“CAT”), horseradish peroxidase, and alkaline phosphatase (“AP”).

Those of skill in the art will understand that the reporter genes utilized in the regulatory region-reporter gene constructs described herein should be easily detected using the methods described herein and that such detection indicates activation of the genetic regulatory region in response to a stimulus (e.g., a drug).

### 5.1.3 Methods of Making Regulatory Region-Reporter Gene Constructs

Regulatory region-reporter gene constructs used to produce the transgenic animals described herein may be made using any method known to those of skill in the art, including well-known molecular biology approaches (e.g., the approaches described in Sambrook et al. Molecular Cloning A Laboratory Manual, 2nd Ed. Cold Spring Lab. Press, December 1989). DNA constructs (e.g., plasmids) can be generated comprising the regulatory region-reporter gene constructs. The nucleic acid sequences corresponding to a chosen regulatory region of a gene (e.g., a c-fos regulatory region) and a chosen reporter gene (e.g., GFP) can be obtained using approaches known in the art (e.g., polymerase chain reaction (PCR)) and subsequently linked to one another by approaches known in the art, such as DNA ligation. Such constructs then can be used in a method of making a transgenic animal (see Section 5.1.3).

In some embodiments, transgenic animals carrying a regulatory region-reporter gene construct are generated using a bacterial artificial chromosome (BAC) or an yeast artificial chromosome (YAC).

### 5.1.4 Methods of Making Transgenic Non-Human Animals

Any transgenic, non-human animal can be used in accordance with the methods described herein. For example, a transgenic animal used in accordance with the methods described herein may be, without limitation, a mouse, a rat, a chicken, a monkey, a cat, a dog, a fish (e.g., a zebrafish), a guinea pig, or a rabbit. In a specific embodiment, the transgenic animals used in accordance with the methods described herein are mice. In another specific embodiment, the transgenic animals used in accordance with the methods described herein are rats. In another specific embodiment, the transgenic animals used in accordance with the methods described herein are monkeys.

Techniques known in the art may be used to introduce a desired regulatory region-reporter gene construct into an animal so as to produce the founder line of transgenic animals. Such techniques include, but are not limited to: pronuclear microinjection (see, e.g., Manipulating the Mouse Embryo, Cold Spring Harbor Laboratory Press, Cold Spring Harbor, N.Y., 1986); nuclear transfer into enucleated oocytes of nuclei from cultured embryonic, fetal or adult cells induced to quiescence (Campbell, et al., 1996, Nature 380:64; Wilmut, et al., Nature 385:810); retrovirus mediated gene transfer into germ lines (Van der Putten et al., Proc. Natl. Acad. Sci. USA 82: 6148-6152, 1985; gene targeting in embryonic stem cells (Thompson et al., Cell 56: 313-321, 1989; electroporation of embryos (Lo, Mol. Cell Biol. 3: 1803-1814, 1983; and sperm-mediated gene transfer (Lavitrano, et al., Cell 57: 717-723, 1989; etc. For a review of techniques for generating transgenic animals, see Gordon, Intl. Rev. Cytol. 115: 171-229, 1989.

In certain embodiments, the transgenic animals used in accordance with the methods described herein have a transgene in all their cells. In other embodiments, the transgenic animals used in accordance with the methods described herein have a transgene in some, but not all of their cells, i.e., the transgenic animals are mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, et al., 1992, Proc. Natl. Acad. Sci. USA 89:6232). The regulatory sequences required for such a cell-type specific activation will depend upon the particular cell type of interest, and will be apparent to those of skill in the art.

Successful generation of a transgenic animal in accordance with the foregoing methods may be measured by methods known in the art, for example, by assessing expression of the transgene using Northern blot or PCR, or by assessing expression or function of a detectable marker (for example, green fluorescent protein) encoded by the transgene. In certain embodiments, the transgene remains stably integrated and is expressed over multiple generations.

The transgenic animals used in accordance with the methods provided herein may be of any age or state of maturity. In certain embodiments, a transgenic animal used in accordance with the methods provided herein has an age in the range of from about 0 months to about 1 month old, from about 1 month to about 3 months old, from about 3 months to about 6 months old, from about 6 months to about 12 months old, from about 6 months to about 18 months old, from about 18 months to about 36 months old, from about 1 year to about 2 years old, from about 1 year to about 5 years old, or from about 5 years to about 10 years old.

In certain embodiments, the transgenic animals used in accordance with the methods provided herein possess a single transgene provided herein. In other embodiments, the transgenic animals used in accordance with the methods provided herein possess more than one transgene provided herein. In a specific embodiment, a transgenic animal used in accordance with the methods provided herein possesses two transgenes provided herein. In another specific embodiment, a transgenic animal used in accordance with the methods provided herein possesses three transgenes provided herein. In another specific embodiment, a transgenic animal used in accordance with the methods provided herein possesses four transgenes provided herein. In another specific embodiment, a transgenic animal used in accordance with the methods provided herein possesses five transgenes provided herein. In another specific embodiment, a transgenic animal used in accordance with the methods provided herein possesses more than five transgenes provided herein.

In certain embodiments, the transgenic animals used in accordance with the methods provided herein possess a characteristic that is useful for the characterization of a test compound being used in a method described herein. In a specific embodiment, a transgenic animal used in accordance with the methods described herein is pregnant. In another specific embodiment, a transgenic animal used in accordance with the methods described herein is young, e.g., the animal is at an age that would be considered young by one of skill in the art for that particular type of animal. In another specific embodiment, a transgenic animal used in accordance with the methods described herein is old, e.g., the animal is at an age that would be considered old by one of skill in the art for that particular type of animal. In another specific embodiment, a transgenic animal used in accordance with the methods described herein is middle-aged, e.g., the animal is at an age that would be considered old by middle-aged of skill in the art for that particular type of animal.

In another specific embodiment, a transgenic animal used in accordance with the methods described herein has been engineered so that it has a certain disease or condition, or is predisposed to developing/acquiring a certain disease or condition, i.e., the transgenic animal represents an animal model for a given disease or condition.

In a specific embodiment, a transgenic animal used in accordance with the methods described herein is an animal model for a disease or condition of the brain. Such animal models include, but are not limited to, animal models for depression (see, e.g., Hua-Cheng et al., 2010, “Behavioral animal models of depression,” Neurosci Bull Aug. 1, 2010, 26(4):327-337; Vollmayr et al., “Neurogenesis and depression: what animal models tell us about the link,” Eur Arch Psychiatry Clin Neurosci 2007, 257:300-303; Cryan et al., “The tail suspension test as a model for assessing antidepressant activity: review of pharmacological and genetic studies in mice,” Neurosci Biobehav Rev 2005, 29: 571-625; Dulawa et al., (2005), “Recent advances in animal models of chronic antidepressant effects: the novelty-induced hypophagia test,” Neurosci. Biobehav. Rev. 29, 771-783; Willner et al., “Chronic mild stress-induced anhedonia: a realistic animal model of depression,” Neurosci Biobehav Rev 1992, 16: 525-534); anxiety (see, e.g., Holmes, (2001), “Targeted gene mutation approaches to the study of anxiety-like behavior in mice,” Neurosci. Biobehav. Rev. 25, 261-273; Blanchard et al. (2001) “Animal models of social stress: effects on behavior and brain neurochemical systems,” Physiol Behav. 73:261-271; Olivier et al., “New animal models of anxiety,” Eur Neuropsychopharmacol. 1994, 4(2):93-102); mood disorders (see, e.g., Cryan et al., “Animal models of mood disorders: Recent developments,” Curr Opin Psychiatry 2007, 20: 1-7); schizophrenia (see, e.g., Marcotte et al., “Animal models of schizophrenia: a critical review,” J Psychiatry Neurosci., 2001, 26(5):395-410); autism (see, e.g., Moy, S. S., and Nadler, J. J., (2008), “Advances in behavioral genetics: mouse models of autism,” Molecular psychiatry 13:14-26); stroke (see, e.g., Beech et al., (2001), “Further characterisation of a thromboembolic model of stroke in the rat,” Brain Res 895(1-2):18-24; Chen et al., (1986) “A model of focal ischemic stroke in the rat: reproducible extensive cortical infarction,” Stroke 17(4):738-43; Alzheimer's disease and dementia (Gotz et al., “Transgenic animal models of Alzheimer's disease and related disorders: histopathology, behavior and therapy,” Mol Psychiatry. 2004, 9(7):664-83; Gotz et al., (2008) “Animal models of Alzheimer's disease and frontotemporal dementia,” Nature Reviews Neuroscience 9:532-544); and brain cancer (see, e.g., WO 2010/138659).

In another specific embodiment, a transgenic animal used in accordance with the methods described herein is an animal model for a human genetic disease or condition. Animal models for use in studying genetic disease have been described (see, e.g., Hardouin and Nagy, “Mouse models for human disease,” Clinical Genetics 57, 237-244 (2000); Yang et al., “Towards a transgenic model of Huntington's disease in a non-human primate,” Nature 453, 921-924 (2008); and Smithies, “Animal models of human genetic diseases,” Trends Genet. 1993 9(4):112-6). In some embodiments, a transgenic animal used in accordance with the methods described herein is engineered to carry a genetic mutation linked to or associated with a heritable cognitive disorder (e.g., autism, schizophrenia, etc). Many genes linked to autism have been discovered and a number of the genetic mouse models were found to be impaired in social and other complex behaviors (Silverman et al., 2010, Nature Reviews 11:490-502). In one embodiment, the imaging techniques described herein (e.g., STP tomography) can be used to characterize the underlying circuit deficits in an animal model for a genetic cognitive disorder. In some embodiments, the methods described herein can be used to identify drugs that can treat or reverse such circuit deficits or restore normal brain function in an animal model for a genetic cognitive disorder.

In another specific embodiment, a transgenic animal used in accordance with the methods described herein is an animal model for cancer. Examples of animal models for cancer in general include, include, but are not limited to, spontaneously occurring tumors of companion animals (see, e.g., Vail & MacEwen, 2000, Cancer Invest 18(8):781-92). Examples of animal models for lung cancer include, but are not limited to, lung cancer animal models described by Zhang & Roth (1994, In-vivo 8(5):755-69) and a transgenic mouse model with disrupted p53 function (see, e.g. Morris et al., 1998, J La State Med Soc 150(4): 179-85). An example of an animal model for breast cancer includes, but is not limited to, a transgenic mouse that over expresses cyclin D1 (see, e.g., Hosokawa et al., 2001, Transgenic Res 10(5):471-8). An example of an animal model for colon cancer includes, but is not limited to, a TCR b and p53 double knockout mouse (see, e.g., Kado et al., 2001, Cancer Res. 61(6):2395-8). Examples of animal models for pancreatic cancer include, but are not limited to, a metastatic model of PancO2 murine pancreatic adenocarcinoma (see, e.g., Wang et al., 2001, Int. J. Pancreatol. 29(1):37-46) and nu-nu mice generated in subcutaneous pancreatic tumors (see, e.g., Ghaneh et al., 2001, Gene Ther. 8(3):199-208). Examples of animal models for non-Hodgkin's lymphoma include, but are not limited to, a severe combined immunodeficiency (“SCID”) mouse (see, e.g., Bryant et al., 2000, Lab Invest 80(4):553-73) and an IgHmu-HOX11 transgenic mouse (see, e.g., Hough et al., 1998, Proc. Natl. Acad. Sci. USA 95(23):13853-8). An example of an animal model for esophageal cancer includes, but is not limited to, a mouse transgenic for the human papillomavirus type 16 E7 oncogene (see, e.g., Herber et al., 1996, J. Virol. 70(3):1873-81). Examples of animal models for colorectal carcinomas include, but are not limited to, Apc mouse models (see, e.g., Fodde & Smits, 2001, Trends Mol Med 7(8):369 73 and Kuraguchi et al., 2000).

In certain embodiments, a transgenic animal used in accordance with the methods described herein is an animal model for a heart condition, diabetes or stroke.

### 5.2 Compounds

Any compound known in the art or later discovered can be utilized (e.g., as a test compound or as a reference compound) in accordance with the methods described herein including, without limitation, small molecules and biological molecules such as antibodies, proteins, peptides, antisense, DNA or RNA, and RNAi.

In some embodiments, the compound is a reference compound that has been shown to produce a therapeutic effect and/or has been characterized for toxicity in clinical studies in a non-human animal or in a human (preferably, human clinical studies). In some embodiments, the compound is a test compound, e.g., a compound whose therapeutic efficacy or toxicity characteristics are not known. In specific embodiments, the compound is a test compound the therapeutic efficacy and/or toxicity characteristics of which it is desirable to predict and/or determine. In certain embodiments, the test compound is an analog or derivative of one or more reference compounds (e.g., 2, 3, 4, 5, or more than 5 compounds, or a mixture of compounds) that have known therapeutic and/or toxicity effects (e.g., for testing whether the test compound has clinical benefits in comparison to the reference compound(s) such as improved therapeutic or toxicity characteristics). In some embodiments, more than one test compound is used in the methods described herein (e.g., 2, 3, 4, 5, 6, 7, 8, 9, 10 or more than 10 compounds). In certain embodiments, the test compound is a mixture of two, three or more compounds. In other embodiments, the test compound is a single compound—not a mixture of compounds.

The compounds used in accordance with the methods described herein can be administered by any means known in the art or indicated for that particular compound. When administered to a transgenic animal, a compound may be administered as a component of a composition that optionally comprises a pharmaceutically acceptable carrier, excipient or diluent. Administration can be systemic or local. Various delivery systems are known (e.g., encapsulation in liposomes, microparticles, microcapsules, capsules) and can be used to administer the compound. Exemplary forms of administration include, without limitation, parenteral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

The compounds used in accordance with the methods described herein may optionally be in the form of a composition comprising the compound and an optional carrier, excipient or diluent. The term “carrier” refers to a diluent, adjuvant (e.g., Freund's adjuvant (complete and incomplete)), excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is a specific carrier when the composition is administered intravenously. Saline solutions and aqueous dextrose and glycerol solutions can also be employed as liquid carriers, particularly for injectable solutions. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents. The compositions and single unit dosage forms can take the form of solutions, suspensions, emulsion, tablets, pills, capsules, powders, sustained-release formulations and the like.

The amount/dose of a compound that will be effective in the successful application of a method described herein can be determined by standard clinical techniques. In vitro or in vivo assays may optionally be employed to help identify optimal dosage ranges. The precise dose to be employed will also depend, e.g., on the route of administration and the type of disease or disorder the compound is indicated for.

In some embodiments, the amount/dose of the test compound used in the described methods is the same (or about the same) as the amount/dose of one or more reference compounds (e.g., a majority or all of the reference compounds). In specific embodiments, the amount/dose of the test compound used in the described methods differs from the amount/dose of one or more reference compounds (e.g., a majority or all of the reference compounds) by less than 75%, 50%, 40%. 30%. 20%, 10%, or 5% of the amount/dose of the reference compound. In other embodiments, the amount/dose of the test compound used in the described methods is not the same as the amount/dose of one or more reference compounds.

In certain embodiments, effects of two or more doses (e.g., 2, 3, 4, 5, 6, 7, 8, 9, 10 or more than 2, 3, 4, 5, 6, 7, 8, 9, 10 amounts/doses) of a compound (e.g., a test compound or a reference compound) are analyzed using described methodology. In particular embodiments, use of two or more doses of a compound allows generation of a dose curve of the compound. In some embodiments, a pharmacomap of the compound is generated at each of the doses. In some aspects, use of more than one dose of two or more compounds and generation of a dose curve for each of the compounds (e.g., a pharmacomap read-out at each of the doses tested) allows differentiation between clinical benefits of the compounds. In one embodiment, a compound is selected based on its ability to achieve a therapeutic effect (the same or an improved therapeutic effect) at a lower dose than that achieved by other compounds. In another embodiment, a compound is selected based on its ability to achieve an improved therapeutic effect at the same or lower dose than that achieved by other compounds. In yet another embodiment, a compound is selected based on its lack of toxicity or lower toxicity at the same or higher dose than that achieved by other compounds. Generation of dose curves for two or more compounds can increase ability to differentiate (e.g., select a compound that is predicted to have the most beneficial clinical outcome) between related drugs (e.g., structurally similar drugs). In some aspects, two or more doses of a test compound can be analyzed in accordance with the described methods, leading to generation of a dose curve for the test compound (e.g., a pharmacomap read-out at each of the doses tested). In some aspects, two or more doses of a reference compound can be analyzed in accordance with the described methods, leading to generation of a dose curve for the reference compound (e.g., a pharmacomap read-out at each of the doses tested). In some embodiments, the pharmacomaps of a test compound or a reference compound at each of the doses tested are stored in a database. In specific embodiments, predicting of clinical benefit of a test compound (e.g., a therapeutic or toxicity benefit) involves determining similarities or differences between the dose curve of the test compound and the dose curve of one or more reference compounds with known clinical characteristics.

Exemplary doses of a compound to be used in accordance with the methods described herein include milligram (mg) or microgram (μg) amounts per kilogram (Kg) of subject or sample weight per day (e.g., from about 1 μg per Kg to about 500 mg per Kg per day, from about 5 μg per Kg to about 100 mg per Kg per day, or from about 10 μg per Kg to about 100 mg per Kg per day). In specific embodiments, a daily dose is at least 0.1 mg, 0.25 mg, 0.5 mg, 0.75 mg, 1.0 mg, 2.0 mg, 5.0 mg, 10 mg, 25 mg, 50 mg, 75 mg, 100 mg, 150 mg, 250 mg, 500 mg, 750 mg, or at least 1 g. In another embodiment, the dosage is a unit dose of about 0.1 mg, 1 mg, 5 mg, 10 mg, 50 mg, 100 mg, 150 mg, 200 mg, 250 mg, 300 mg, 350 mg, 400 mg, 500 mg, 550 mg, 600 mg, 650 mg, 700 mg, 750 mg, 800 mg or more. In another embodiment, the dosage is a unit dose that ranges from about 0.1 mg to about 1000 mg, 1 mg to about 1000 mg, 5 mg to about 1000 mg, about 10 mg to about 500 mg, about 150 mg to about 500 mg, about 150 mg to about 1000 mg, 250 mg to about 1000 mg, about 300 mg to about 1000 mg, or about 500 mg to about 1000 mg. In another embodiment, a non-human animal (e.g., a transgenic animal) is administered one or more doses of an effective amount of a compound or a composition, wherein the effective amount is not the same for each dose.

In certain embodiments, a compound used in accordance with the methods described herein is administered once to a non-human animal (e.g., a transgenic animal). In certain embodiments, a compound used in accordance with the methods described herein is administered more than once to a non-human animal (e.g., a transgenic animal), e.g., the compound is administered twice, three times, four times, five times, six times, seven times, eight times, nine times, ten times, or more than ten times.

In certain embodiments, a compound used in accordance with the methods described herein is administered continuously to a non-human animal (e.g., a transgenic animal), i.e., the animal is fitted with a mechanism (e.g., a pump, an i.v., a catheter, or another appropriate mechanism known to those of skill in the art) that allows for continuous infusion of the compound to the animal for a desired period of time.

In certain embodiments, a compound used in accordance with the methods described herein is administered to a non-human animal (e.g., a transgenic animal) more than once, with a specified period of time in between the administrations. For example, a compound may be administered to a non-human animal (e.g., a transgenic animal) every 5 minutes, every 10 minutes, every 20 minutes, every 30 minutes, hourly, every 2 hours, every 3 hours, every 4 hours, every 5 hours, every 6 hours, every 7 hours, every 8 hours, every 9 hours, every 10 hours, ever 11 hours, every 12 hours, every 24 hours (i.e., daily at the same time each day), weekly, or monthly for a desired period of time. In certain embodiments, a compound used in accordance with the methods described herein may be administered to a non-human animal (e.g., a transgenic animal) more than once, with a specified period of time in between the administrations, wherein said compound is administered every 1-5 minutes, every 5-10 minutes, every 10-20 minutes, every 20-30 minutes, every 30-60 minutes, every 1-2 hours, every 2-4 hours, every 4-8 hours, every 8-12 hours, every 12-16 hours, every 16-20 hours, every 20-24 hours, every 1-2 days, every 1-3 days, every 2-4 days, every 5-7 days, every 7-14 days, every 14-21 days, or every 21-28 days.

In certain embodiments, when a compound used in accordance with the methods described herein is administered to a non-human animal (e.g., a transgenic animal) so as to analyze the animal's acute response to a compound, the compound may be administered as a single dose, or in multiple doses, followed shortly thereafter (e.g., within hours) by analysis using the methods described herein.

In certain embodiments, when a compound used in accordance with the methods described herein is administered to a non-human animal (e.g., a transgenic animal) so as to analyze the animal's long-term response to a compound, the compound may be administered as a single dose, or in multiple doses, followed by analysis using the methods described herein at a later period of time, e.g., the analysis may be performed days, weeks, or months after the initial administration of the compound.

In some embodiments, a compound used in accordance with the methods described herein is administered repeatedly or chronically to a non-human animal (e.g., a transgenic animal) for days (e.g., 2 days, 3 days, 4 days, 5 days, 6 days, 7 days, 8 days, 9 days, 10 days, 11 days, 12 days, or 13 days), weeks (e.g., 2 weeks, 3 weeks, 4 weeks, 5 weeks, 6 weeks, or 7 weeks) or months (e.g., 2 months, 3 months, 4 months, 5 months, 6 months, 7 months, 8 months, 9 months, 10 months, 11 months, 12 months, 18 months, 24 months, 30 months, or 36 months), followed by analysis using the methods described herein after the last administration of the compound. In specific embodiments, a pharmacompap generated by such method would represent a pharmacomap of a chronic effect. In particular embodiments, a compound used in accordance with the methods described herein is administered repeatedly or chronically to a non-human animal (e.g., a transgenic animal) for at least 1 week, at least 2 weeks, at least 3 weeks, at least 1 month, at least 2 months, at least 3 months, at least 4 months, at least 5 months, at least 6 months, at least 8 months, at least 10 months, or at least 1 year, followed by analysis using the methods described herein after the last administration of the compound.

In a specific embodiment, the compound(s) used in accordance with the methods described herein is a compound that is capable of crossing the blood-brain barrier. In another specific embodiment, a compound(s) used in accordance with the methods described herein may be incapable of crossing the blood-brain barrier naturally, but may be made to cross the blood-brain barrier using approaches known to those of skill in the art.

Physical methods of transporting a compound across the blood-brain barrier include, but are not limited to, circumventing the blood-brain barrier entirely, or by creating openings in the blood-brain barrier. Circumvention methods include, but are not limited to, direct injection into the brain (see, e.g., Papanastassiou et al., Gene Therapy 9: 398-406 (2002)) and implanting a delivery device in the brain (see, e.g., Gill et al., Nature Med. 9: 589-595 (2003); and Gliadel Wafers™, Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416).

Lipid-based methods of transporting a compound across the blood-brain barrier include, but are not limited to, encapsulating the compound in liposomes that are coupled to antibody binding fragments that bind to receptors on the vascular endothelium of the blood-brain barrier (see, e.g., U.S. Patent Application Publication No. 20020025313), and coating the compound in low-density lipoprotein particles (see, e.g., U.S. Patent Application Publication No. 20040204354) or apolipoprotein E (see, e.g., U.S. Patent Application Publication No. 20040131692).

Receptor and channel-based methods of transporting a compound across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), inhibiting ABC drug transporters (see, e.g., U.S. Patent Application Publication No. 2003/0073713); coating compounds with a transferrin and modulating activity of the one or more transferrin receptors (see, e.g., U.S. Patent Application Publication No. 2003/0129186), and cationizing the compounds (see, e.g., U.S. Pat. No. 5,004,697).

In another specific embodiment, the compound(s) used in accordance with the methods described herein is a reference compound, which is known to be effective in the treatment of a brain disease or disorder including, without limitation, a psychotic disease or disorder, a mania, anxiety, depression, schizophrenia, bipolar disorder, multiple personality disorder, Alzheimer's disease, dementia, cancers of the brain, stroke, traumatic brain injury (TBI), and migraines.

In another specific embodiment, the compound(s) used in accordance with the methods described herein is a reference compound, which is known to be effective in the treatment of a psychotic disease or disorder, i.e., the compound is an anti-psychotic compound. A non-limiting list of anti-psychotic compounds includes Chlorpromazine (Thorazine), Haloperidol (Haldol), Perphenazine (Trilafon), Fluphenazine (Permitil), Clozapine (Clozaril), Risperidone (Risperdal), Olanzapine (Zyprexa), Quetiapine (Seroquel), Ziprasidone (Geodon), Aripiprazole (Abilify), Paliperidone (Invega), chlorprothixene (Taractan), loxapine (Loxitane), mesoridazine (Serentil), molindone (Lidone, Moban), olanzapine (Zyprexa), pimozide (Orap), thioridazine (Mellaril), thiothixene (Navane), trifluoperazine (Stelazine), and trifluopromazine (Vesprin).

In another specific embodiment, the compound(s) used in accordance with the methods described herein is a reference compound, which is known to be effective in the treatment of depression, i.e., the compound is an anti-depressant compound. A non-limiting list of anti-depressant compounds includes serotonin reuptake inhibitors (SSRIs) such as Fluoxetine (Prozac), Citalopram (Celexa), Sertraline (Zoloft), fluvoxamine (Luvox) Paroxetine (Paxil), and Escitalopram (Lexapro); serotonin and norepinephrine reuptake inhibitors (SNRIs) such as venlafaxine (Effexor) and duloxetine (Cymbalta); bupropion (Wellbutrin); amitriptyline (Elavil); amoxapine (Asendin); clomipramine (Anafranil); desipramine (Norpramin, Pertofrane); doxepin (Adapin, Sinequan); imipramine (Tofranil); tricyclics; tetracyclics; and monoamine oxidase inhibitors (MAOIs) such as isocarboxazid (Marplan); phenelzine (Nardil); and tranylcypromine (Parnate).

In another specific embodiment, the compound(s) used in accordance with the methods described herein is a reference compound, which is known to be effective in the treatment of anxiety, i.e., the compound is an anti-anxiety compound. A non-limiting list of anti-anxiety compounds includes alprazolam (Xanax), buspirone (BuSpar), chlordiazepoxide (Librax, Libritabs, Librium), clonazepam (Klonopin), clorazepate (Azene, Tranxene), diazepam (valium), halazepam (Paxipam), lorazepam (Ativan), oxazepam (Serax), and prazepam (Centrax).

In another specific embodiment, the compound(s) used in accordance with the methods described herein is a reference compound, which is known to be effective in the treatment of a mania, i.e., the compound is an anti-manic compound. A non-limiting list of anti-anxiety compounds includes carbamazepine (Tegretol), divalproex sodium (Depakote), gabapentin (Neurontin), lamotrigine (Lamictal), lithium carbonate (Eskalith, Lithane, Lithobid), lithium citrate (Cibalith-S), and topimarate (Topamax).

In another specific embodiment, the compound(s) used in accordance with the methods described herein is a reference compound, which is known to be effective in the treatment of Alzheimer's disease. A non-limiting list of compounds used in the treatment of Alzheimer's disease includes, without limitation, donepezil (Aricept), galantamine (Razadyne), memantine (Namenda), rivastigmine (Exelon), and tacrine (Cognex).

In another specific embodiment, the compound(s) used in accordance with the methods described herein is a reference compound, which is known to be effective in the treatment of a liver disease or disorder. In another specific embodiment, the compound(s) used in accordance with the methods described herein is a reference compound, which is known to be effective in the treatment of a disease or disorder of a tissue or organ of the body other than the brain and/or liver, such as the pancreas, the heart, the spleen, the stomach, the lung, the small intestines, the large intestines, the kidneys, the bladder, the ovaries, the testes, or the prostate.

Other compounds that may be used in accordance with the methods described herein include, without limitation, nucleoside analogs (e.g., zidovudine, acyclovir, gangcyclovir, vidarabine, idoxuridine, trifluridine, and ribavirin), foscarnet, amantadine, peramivir, rimantadine, saquinavir, indinavir, ritonavir, alpha-interferons and other interferons, AZT, zanamivir (Relenza®), oseltamivir (Tamiflu®), Amoxicillin, Amphothericin-B, Ampicillin, Azithromycin, Bacitracin, Cefaclor, Cefalexin, Chloramphenicol, Ciprofloxacin, Colistin, Daptomycin, Doxycycline, Erythromycin, Fluconazol, Gentamicin, Itraconazole, Kanamycin, Ketoconazole, Lincomycin, Metronidazole, Minocycline, Moxifloxacin, Mupirocin, Neomycin, Ofloxacin, Oxacillin, Penicillin, Piperacillin, Rifampicin, Spectinomycin, Streptomycin, Sulbactam, Sulfamethoxazole, Telithromycin, Temocillin, Tylosin, Vancomycin, and Voriconazole.

Other compounds that may be used in accordance with the methods described herein include, without limitation, acivicin; anthracyclin; anthramycin; azacitidine (Vidaza); bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); carboplatin; chlorambucil; cisplatin; cytarabine (Ara-C); daunorubicin hydrochloride; decitabine (Dacogen); demethylation agents, docetaxel; doxorubicin; EphA2 inhibitors; etoposide; fazarabine; fluorouracil; gemcitabine; histone deacetylase inhibitors (HDACs); interleukin II (including recombinant interleukin II, or rIL2), interferon alpha; interferon beta; interferon gamma; lenalidomide (Revlimid); anti-CD2 antibodies (e.g., siplizumab (MedImmune Inc.; International Publication No. WO 02/098370, which is incorporated herein by reference in its entirety)); melphalan; methotrexate; mitomycin; oxaliplatin; paclitaxel; puromycin; riboprine; spiroplatin; tegafur; teniposide; vinblastine sulfate; vincristine sulfate; vorozole; zeniplatin; zinostatin; zorubicin hydrochloride; angiogenesis inhibitors; antisense oligonucleotides; apoptosis gene modulators; apoptosis regulators; BCR/ABL antagonists; beta lactam derivatives; casein kinase inhibitors (ICOS); estrogen agonists; estrogen antagonists; glutathione inhibitors; HMG CoA reductase inhibitors; immunostimulant peptides; insulin-like growth factor-1 receptor inhibitor; interferon agonists; interferons; interleukins; lipophilic platinum compounds; matrilysin inhibitors; matrix metalloproteinase inhibitors; mismatched double stranded RNA; nitric oxide modulators; oligonucleotides; platinum compounds; protein kinase C inhibitors, protein tyrosine phosphatase inhibitors; purine nucleoside phosphorylase inhibitors; raf antagonists; signal transduction inhibitors; signal transduction modulators; translation inhibitors; tyrosine kinase inhibitors; and urokinase receptor antagonists.

Other compounds that may be used in accordance with the methods described herein include, without limitation, anti-angiogenic agents including proteins, polypeptides, peptides, conjugates, antibodies (e.g., human, humanized, chimeric, monoclonal, polyclonal, Fvs, ScFvs, Fab fragments, F(ab)2 fragments, and antigen-binding fragments thereof) such as antibodies that specifically bind to TNF-α, nucleic acid molecules (e.g., antisense molecules or triple helices), organic molecules, inorganic molecules, and small molecules that reduce or inhibit angiogenesis; anti-inflammatory agents including non-steroidal anti-inflammatory drugs (NSAIDs) (e.g., celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™)), steroidal anti-inflammatory drugs (e.g., glucocorticoids, dexamethasone (DECADRON™), corticosteroids (e.g., methylprednisolone (MEDROL™)), cortisone, hydrocortisone, prednisone (PREDNISONE™ and DELTASONE™), and prednisolone (PRELONE™ and PEDIAPRED™)), anticholinergics (e.g., atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)).

Other compounds that may be used in accordance with the methods described herein include, without limitation, alkylating agents, nitrosoureas, antimetabolites, anthracyclins, topoisomerase II inhibitors, and mitotic inhibitors. Alkylating agents include, but are not limited to, busulfan, cisplatin, carboplatin, cholormbucil, cyclophosphamide, ifosfamide, decarbazine, mechlorethamine, mephalen, and themozolomide. Nitrosoureas include, but are not limited to carmustine (BCNU) and lomustine (CCNU). Antimetabolites include but are not limited to 5-fluorouracil, capecitabine, methotrexate, gemcitabine, cytarabine, and fludarabine. Anthracyclins include but are not limited to daunorubicin, doxorubicin, epirubicin, idarubicin, and mitoxantrone. Topoisomerase II inhibitors include, but are not limited to, topotecan, irinotecan, etopiside (VP-16), and teniposide. Mitotic inhibitors include, but are not limited to taxanes (paclitaxel, docetaxel), and the vinca alkaloids (vinblastine, vincristine, and vinorelbine).

In specific embodiments, the compounds that are used in accordance with the methods described herein are any one or more of the compounds described in the Examples. In some embodiments, the compounds that are used in accordance with the methods described herein are any one or more of the compounds described in Examples 9, 10, 11 and/or 12. In a specific embodiment, the compounds that are used in accordance with the methods described herein are any one or more of the compounds described in Example 11.

### 5.3 Preparation of Animals for Analysis

In some embodiments, the non-human animal used in accordance with the methods described herein is prepared for a procedure to harvest/remove a tissue(s) without sacrificing the animal using techniques known to one skilled in the art. In other embodiments, the non-human animals (e.g., transgenic animals) used in accordance with the methods described herein is sacrificed using any methods known in the art. In certain embodiments, a non-human animal used in accordance with the methods described herein is sacrificed in a manner that ensures that the tissue of the animal will be suitable for a desired type of analysis. For example, if the tissue of the non-human animal to be analyzed is the brain, then the animal is to be sacrificed in a manner that will do disturb/disrupt the tissue of the brain. In a specific embodiment, the sacrificed non-human animals used in accordance with the methods are transgenic animals that possess one or more transgenes. In another specific embodiment, the sacrificed animals used in accordance with the methods are not transgenic animals.

In certain embodiments, the non-human animals used in accordance with the methods provided herein are sacrificed using intracardiac perfusion. Briefly, a non-human animal, e.g., a mouse, may be sacrificed by intracardiac perfusion as follows: the non-human animal is anesthetized by an injection (e.g., an intraperitoneal injection) with an anaesthetic (e.g., ketamine and xylazine); once deep anesthesia is attained, the animal is pinned in dorsal recumbency, the chest is quickly opened, and the right atrium cut with scissors. A needle is placed in the left ventricle and a incision is made in the right ventricle. Next, saline flushed into the heart with the needle for a period of time sufficient to kill the non-human animal (e.g., about 4 minutes). Next, paraformaldehyde (e.g., 4% paraformaldehyde) is flushed into the heart until the body becomes stiff. In a specific embodiment, when the tissue to be analyzed in accordance with the methods provided herein is brain tissue, the animal used in the method is sacrificed using intracardiac perfusion.

Other methods of sacrificing non-human animals include, without limitation, injection (e.g., intraperitoneal injection) of the animal with barbiturates or other suitable euthanasia solutions; exposure of the animal to an atmosphere of, e.g., carbon dioxide, methoxyflurane, or halothane; and cervical dislocation of the animal.

Once a non-human animal is sacrificed, the tissue of the animal desired for analysis (e.g., brain tissue) can be obtained for use—for example, if the tissue desired to be analyzed is brain tissue, the animal can subsequently be decapitated and the brain tissue isolated. Any tissue desired for analysis can be harvested from the sacrificed non-human animal(s) including, without limitation, tissues from the brain, the liver, pancreas, the heart, the spleen, the stomach, the lung, the small intestines, the large intestines, the kidneys, the bladder, the ovaries, the testes, or the prostate. In certain embodiments, multiple tissues are obtained from a non-human animal after it has been sacrificed, e.g., the brain, liver, and/or other tissues are isolated from the animal. In some embodiments, an entire organ is harvested, e.g., whole brain, whole liver, whole heart (or any other organ of the body of the non-human animal). In other embodiments, a piece, part or section of an organ(s) are obtained from a non-human animal.

The tissues then can be post-fixed in a suitable fixative (e.g., 4% paraformaldehyde) for several hours or longer (e.g., overnight or for several days to weeks). In certain embodiments, once fixed, the tissues can be stored (e.g., for hours, days, weeks, months, or longer) under suitable conditions (e.g., at 4° C.), until ready for analysis.

### 5.4 Imaging

Tissues obtained from the non-human animals (e.g., transgenic animals) used in accordance with the methods described herein can be imaged using any method known to those of skill in the art and suitable based on the gene expression being detected (e.g., methods suitable based on the reporter gene used in the transgene of the transgenic animal).

In some embodiments, imaging of non-human animals (e.g., to detect expression of fluorescent or enzymatic reporter genes) can be done by light microscopy. In other embodiments, imaging of non-human animals (e.g., to detect native gene expression) can be done by light microscopy after the native gene expression is visualized by immunohistochemistry or in situ hybridization.

In certain embodiments, the imaging technique used in the methods described herein provides single cell resolution of cells in the tissue. In specific embodiment, the imaging technique used provides single cell resolution of cells expressing a transgene.

In certain embodiments, non-human animals are imaged using two-photon cytometry (see, e.g., Ragan et al. “High-resolution whole organ imaging using two-photon tissue cytometry,” Journal of biomedical optics 12, 014015 (2007)).

In a specific embodiment, the tissues are imaged via serial two-photon (STP) tomography, as described herein (see, e.g., Section 5, supra, and Sections 6.1 and 6.8, infra; Ragan et al., Nature Methods 9(3):255-258 (2012)). Briefly, a fixed agar-embedded non-human animal tissue (e.g., mouse brain) is placed in a water bath on XYZ stage under the objective of a two-photon microscope (see, e.g., Denk et al., “Two-photon laser scanning fluorescence microscopy,” Science 248, 73-76 (1990)) and imaging parameters are entered in the operating software of the microscope. Once the parameters are set, the instrument works fully automatically: 1) the XYZ stage moves the brain under the objective so that an optical section (or an optical Z-stack) is imaged as a mosaic of fields of view (FOVs), 2) a built-in vibrating blade microtome mechanically cuts off a tissue section from the top, and 3) the steps of overlapping optical and mechanical sectioning are repeated until the whole dataset is collected. Sectioning by vibrating blade microtome allows the use of tissues (e.g., brains) prepared by simple procedures of formaldehyde fixation and agar embedding, which have minimal detrimental effects on fluorescence and tissue morphology. High-speed galvanometric scanning enables fast imaging and switching between different sampling resolutions for different experiments. Thus, the use of two-photon microscopy allows deep tissue imaging, which is advantageous for focusing below the surface to obtain undisturbed optical sections and to collect high-resolution Z-stacks between sectioning steps. STP microscopy is generally described in U.S. Pat. No. 7,724,937, which is incorporated herein by reference in its entirety.

Other imaging techniques that can be used to image the tissues of the non-human animals (e.g., transgenic animals) described herein, include all-optical histology (see, e.g., Tsai, P. S., et al. All-optical histology using ultrashort laser pulses. Neuron 39, 27-41 (2003)), robotized wide-field fluorescence microscopy of mounted serial brain sections (see, e.g., Lein, E. S., et al. Genome-wide atlas of gene expression in the adult mouse brain. Nature 445, 168-176 (2007)), light-sheet fluorescence microscopy (LSFM; also known as selective-plane illumination microscopy (SPIM) (see, e.g., Huisken, J., Swoger, J., Del Bene, F., Wittbrodt, J. & Stelzer, E. H. Optical sectioning deep inside live embryos by selective plane illumination microscopy. Science 305, 1007-1009 (2004)), OCPI light-sheet microscopy, ultramicroscopy (see, e.g., Dodt, H. U., et al. Ultramicroscopy: three-dimensional visualization of neuronal networks in the whole mouse brain. Nature methods 4, 331-336 (2007)), and micro-optical sectioning tomography (MOST) (see, e.g., Li, A., et al. Micro-optical sectioning tomography to obtain a high-resolution atlas of the mouse brain. Science 330, 1404-1408 (2011)) which is also known as knife-edge scanning microscopy (see, e.g., Mayerich, D., Abbott, L. & McCormick, B. Knife-edge scanning microscopy for imaging and reconstruction of three-dimensional anatomical structures of the mouse brain. Journal of microscopy 231, 134-143 (2008)).

In another embodiment, the imaging technique used in the methods described herein is in situ hybridization of particular genes of interest (e.g., immediate early genes or reporter genes). This technique can be used to detect, e.g., the non-coding region of RNAs.

### 5.5. Pharmacomaps; Computer Processing and Analysis; Databases of Pharmacomaps

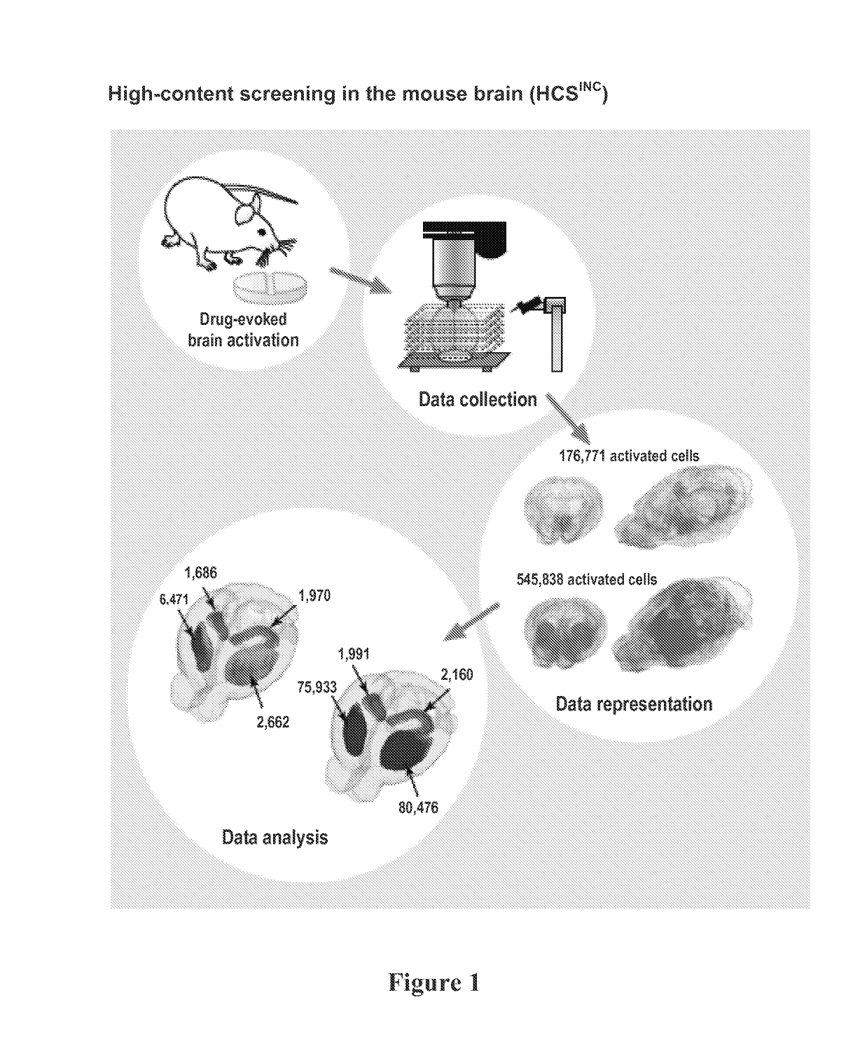

FIG. 1 illustrates operations for a pharmacomap data representation and analysis process. In this example, data related to compound-evoked activation of a non-human animal tissue in response to test compounds is collected and analyzed. Computationally identified activation of the animal tissue is visualized in a multiple-dimension representation. From this multiple-dimension representation, a pharmacomap is generated. A pharmacomap of the test compound or a reference compound represents a unique pattern of compound-evoked activation in a non-human animal tissue in response to the test compound or reference compound, respectively. Comparison and analysis of pharmacomaps of different compounds, e.g., pharmacomap of a reference compound with that of other reference compounds, or pharmacomap of a reference compound with that of a test compound, can provide insight into the possible effects of such compounds based on the known effects of the compared reference pharmacomaps. For example, comparison and analysis of pharmacomaps of test compounds can provide insight into the possible effects of test compounds based on the known effects of the compared reference pharmacomaps.

As an illustration, a test compound (e.g., a candidate drug) is administered on a transgenic animal (e.g., a mouse). A tissue (e.g., brain tissue) of the transgenic animal is harvested for analysis. The harvested tissue is imaged, and a computational analysis of the tissue images is performed to identify activated cells in the tissue. A multiple dimension, e.g., three-dimension (3D), data representation of the compound-evoked activation is generated. Statistical methods analyze the data representation of the compound-evoked activation to identify activated regions in the tissue. A pharmacomap data representation is generated for the test compound. The generated pharmacomap data representation is then compared with pharmacomap data representations of reference compounds that have known effects for use in predicting possible effects of the test compound.

In other embodiments, a reference compound that has a known clinical effect is administered on a transgenic animal (e.g., a mouse). A tissue (e.g., brain tissue) of the transgenic animal is harvested for analysis. The harvested tissue is imaged, and a computational analysis of the tissue images is performed to identify activated cells in the tissue. A multiple dimension, e.g., three-dimension (3D), data representation of the compound-evoked activation is generated. Statistical methods analyze the data representation of the compound-evoked activation to identify activated regions in the tissue. A pharmacomap data representation is generated for the reference compound. The generated pharmacomap data representation can then be deposited into a database (e.g., a database of reference compound pharmacomaps).

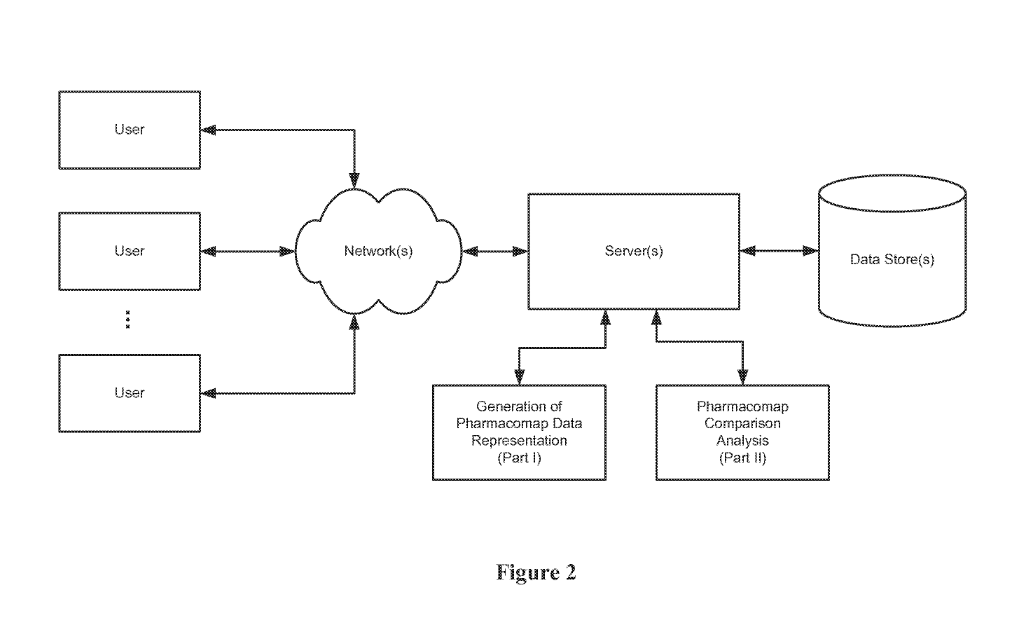

FIG. 2 depicts a computer-implemented environment wherein users can interact with pharmacomap data representation and analysis systems hosted on one or more servers through a network. The pharmacomap data representation and analysis systems can assist the users to generate a pharmacomap data representation of a test compound. Correlations between the pharmacomaps of the reference compounds and the known therapeutic or toxicity effects of the reference compounds may be determined. The possible effects of the test compound can then be predicted based on the comparison of the pharmacomaps of the test compound and the reference compounds.

As shown in FIG. 2, the users can interact with the pharmacomap data representation and analysis systems through a number of ways, such as over one or more networks. One or more servers accessible through the network(s) can host the pharmacomap data representation and analysis systems. The server(s) can also contain or have access to one or more data stores for storing data to be analyzed by the pharmacomap data representation and analysis systems as well as any intermediate or final data generated by the pharmacomap data representation and analysis systems.

The pharmacomap data representation and analysis systems can be a web-based analysis tool that provides users flexibility and functionality for performing pharmacomap data representation and analysis. It should be understood that the system could also be provided on a stand-alone computer for access by a user.

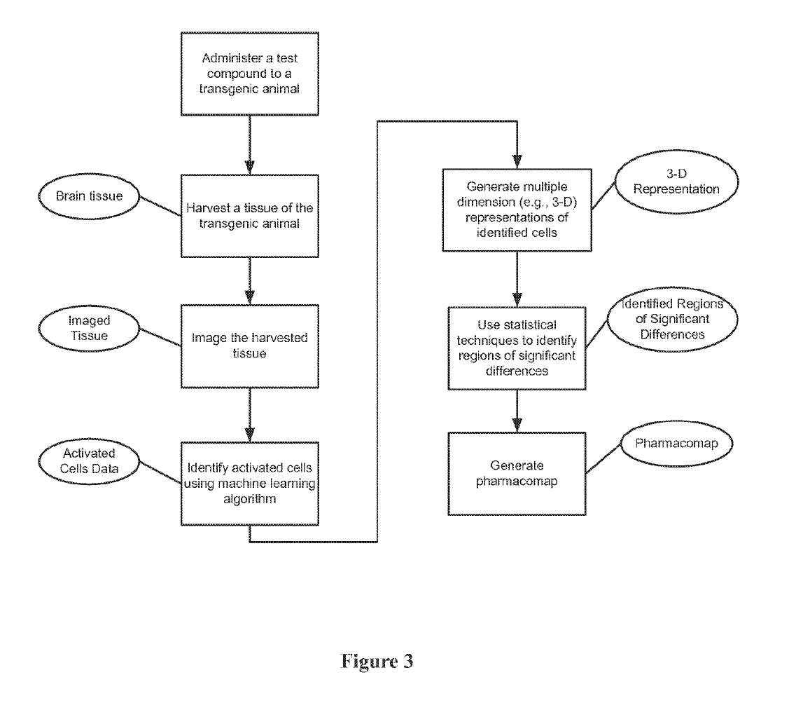

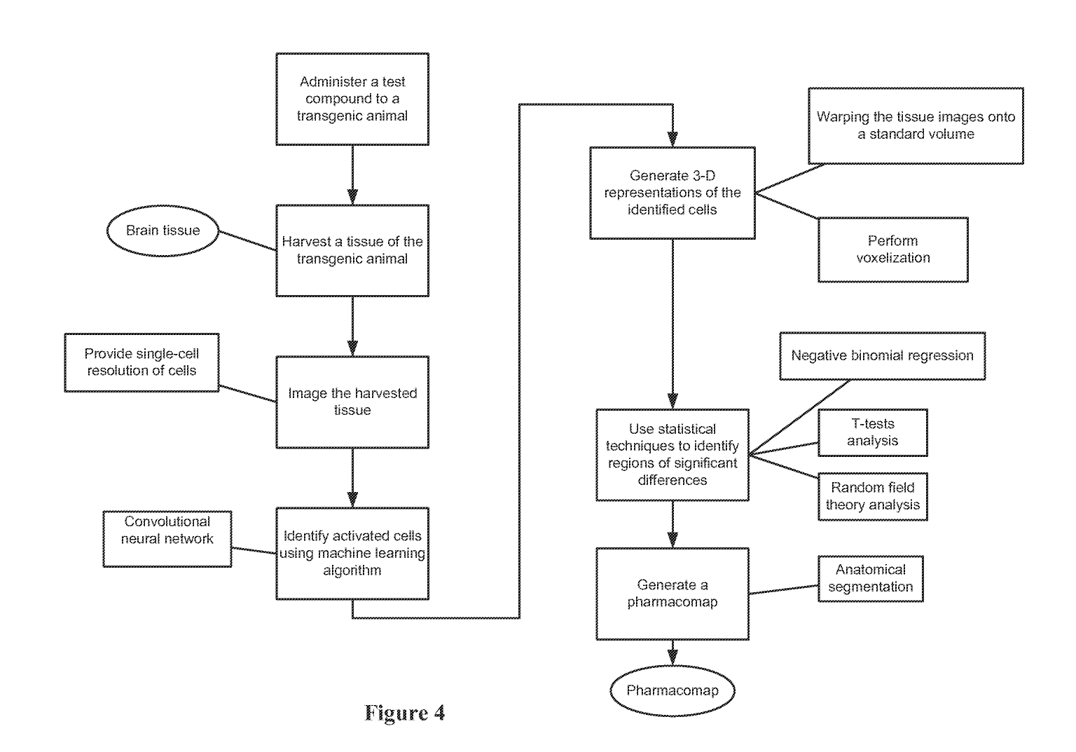

FIG. 3 illustrates operations for generating pharmacomap data representations. In this example, a test compound is administered to a transgenic animal, and a tissue harvested from the transgenic animal is imaged to capture activation of cells in response to the test compound. Multiple dimension (e.g., 3D) representations are generated for activated cells that are identified, and statistical analyses are performed to identify regions of significant differences. Pharmacomap data representations are generated to identify anatomical tissue regions activated in response to the test compound.

Specifically, the test compound is administered to the transgenic animal that includes a genetic regulatory region to control expression of a detectable, e.g., fluorescent, reporter gene sequence. For example, the transgenic animal that expresses green fluorescent protein (GFP) as a surrogate marker from specific IGE promoters, such as c-fos and Arc promoters (e.g., a transgenic c-fos-GFP mouse) could be used for administering the test compound. A tissue (e.g., a brain tissue) harvested from the transgenic animal is imaged using an imaging technique, such as serial two-photon (STP) tomography for generating a serial two-dimensional section imaging dataset. For example, the images of the tissue may be reconstructed as a series of two-dimensional sections for computational detection of activated cells. Data of the imaged tissue is analyzed computationally, and cells activated in response to the test compound can be identified using a machine learning algorithm. Data of activated cells are used to generate multiple dimension (e.g., 3D) representations of identified cells. Various statistical techniques can be used to analyze the generated multiple dimension (e.g., 3D) representation to identify regions of significant differences between control and compound-activated tissues. Based on the identified regions of significant differences, pharmacomap data representations can be generated for multiple purposes, such as predicting possible therapeutic or toxicity effects of the test compound.

It should be understood that similar to the other process flows contained herein, the operations provided in FIG. 3 can be modified or augmented to accomplish the overall goal. As an illustration, FIG. 4 illustrates additional techniques that can be used to generate pharmacomap data representations. For example, harvested tissue (e.g., a brain tissue) harvested from a transgenic animal can be imaged using different imaging techniques. More particularly, the harvested tissue can be imaged using STP tomography, Allen institute serial microscopy, all-optical histology, robotized wide-field fluorescence microscopy, light-sheet fluorescence microscopy, OCPI light-sheet, micro-optical sectioning tomography, etc. For example, STP tomography can be used to integrate fast two-photon imaging and vibratome-based sectioning of a fixed, agar-embedded animal tissue.

Further, different machine learning algorithms, such as a convolutional neural network algorithm support vector machines, random forest classifiers, and boosting classifiers, can be used for automated detection of the activated cells. For example, two-dimensional (e.g., 2D) section images of the harvested tissue can each include a mosaic of individual fields of view, e.g., image tiles. A machine learning algorithm, e.g., a convolutional neural network algorithm, may be trained to detect activated cells and detect activated cells automatically after being trained. For example, the machine learning algorithm may be trained from ground truth data based on many randomly selected image tiles marked up by human observers. Human validation of the training or the automatic detection of the activated cells may be performed. For further technical details of the convolutional neural network algorithm, reference is made to the U.S. Patent Publication No. 2010/0183217, entitled “Method And Apparatus For Image Processing,” filed Apr. 24, 2008, which is incorporated by reference in its entirety.

Once the activated cells are computationally identified through the machine learning algorithms, a multiple dimension (e.g., 3D) representation (e.g., of intensity centroids) is generated for the identified cells. The tissue images are warped onto a standard volume of continuous tissue space to register information associated with the identified cells within the tissue space. For example, the 2D section images of the tissue may be reconstructed in 3D and warped onto a 3D reference brain volume on an auto-fluorescence channel using mutual information as a constraint, and tissue region labels are also warped using the same warping parameters before being resampled to original x,y,z resolutions for performing regional counting. Information associated with the activated cells (e.g., c-fos-GFP data) is registered onto the reference brain volume to create a multiple dimension (e.g., 3D) representation of a distribution of the activated cells. The 3D representation of a distribution of the activated cells may be voxelized to generate discrete digitization of the tissue space, where different voxel sizes (e.g., 50 μm³) can be used. For example, the tissue space may be voxelized as an evenly spaced grid of 450×650×300 voxels, each voxel of size 20×20×50 μm³.

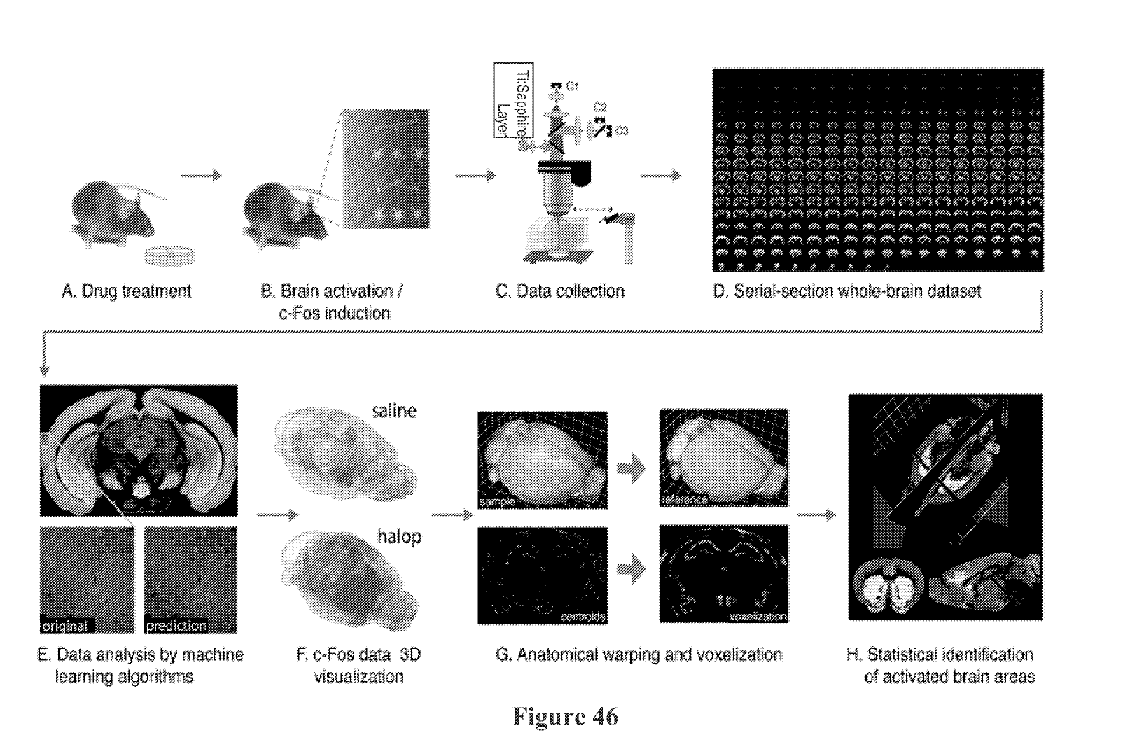

Various statistical techniques can be used to identify regions of significant differences between control and compound-activated tissues, including a negative binomial regression analysis, t-tests and random field theory (RFT) analysis. For example, an initial comparison between different tissues can be performed at a voxel level using a negative binomial regressions with a count data of activated cells as a response variable and a N factor group status as an explanatory variable. A proper false discovery rate (e.g., 0.01) may be set to correct type I errors, under an assumption that the voxels have some level of positive correlation with each other. As another example, comparison of control and compound-activated tissues is carried out with a set oft-tests applied to each voxel, which identifies “hotspots” of differences. The hotspot regions can be evaluated by statistical analyses used for functional tissue imaging, such as order statistics based on RFT analysis which takes advantage of the inherent correlation structure between neighboring voxels to reduce the thresholds required for determining significance in the tests between groups. For example, the identified regions of statistically significant differences may be anatomically annotated, using both the segmentation of a magnetic-resonant-imaging (MRI) atlas (e.g., 62 region segmentation) and visual analysis of the corresponding raw image data. Statistical comparison of activated cells in anatomically segmented regions may be performed. A more detailed example for generating pharmacomap data representations is shown in FIG. 46 and described in Section 6.8, Example 8.

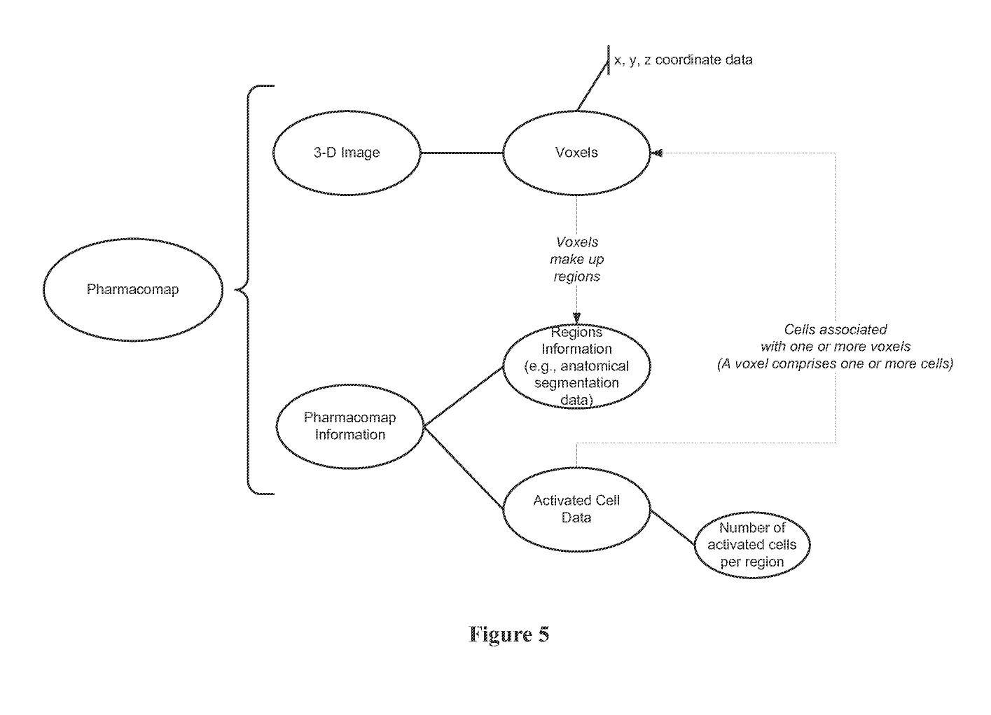

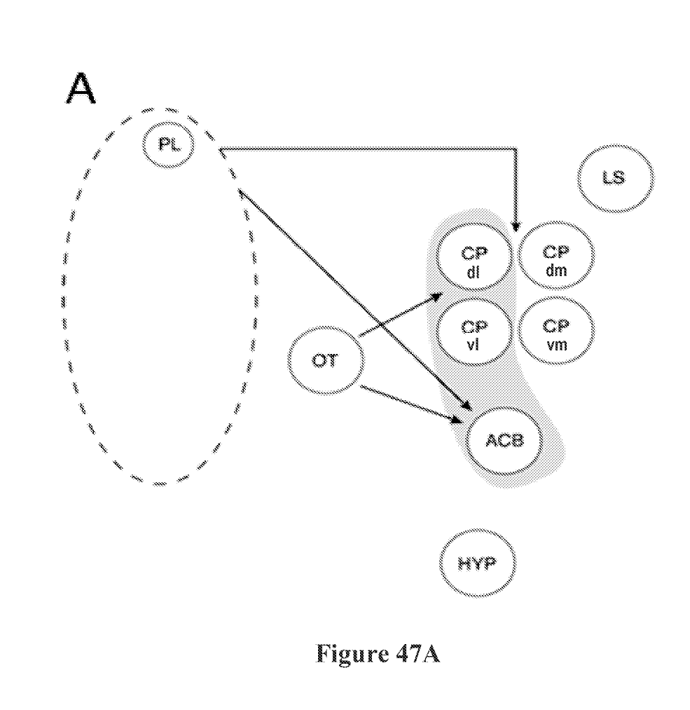

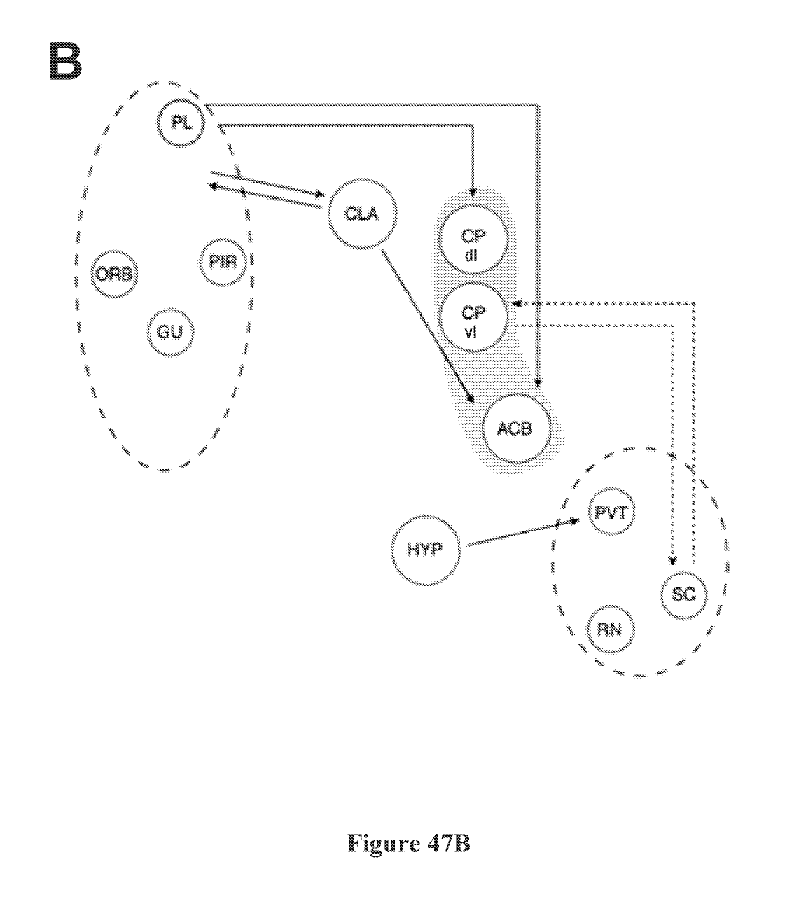

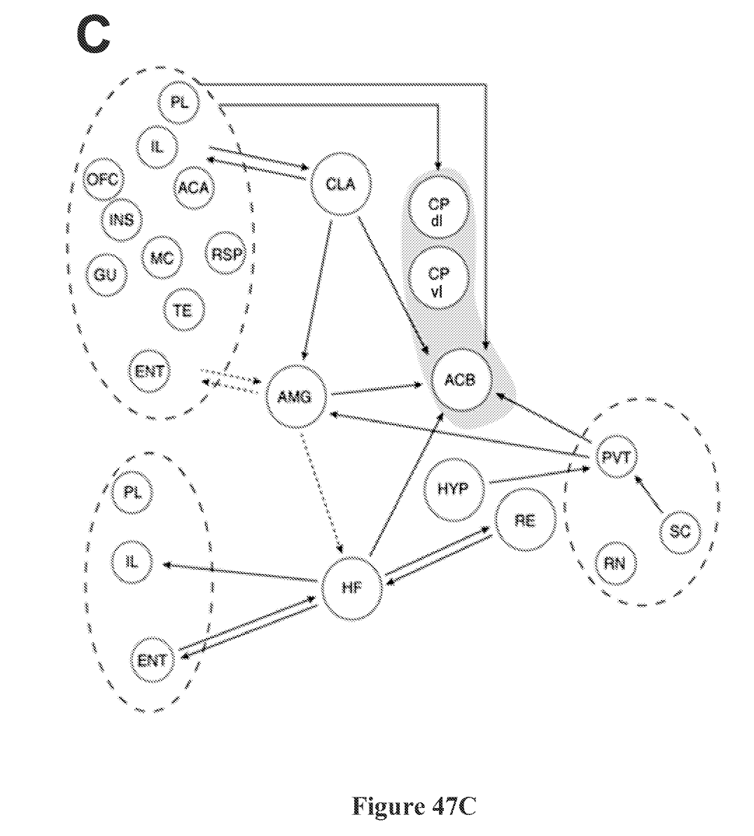

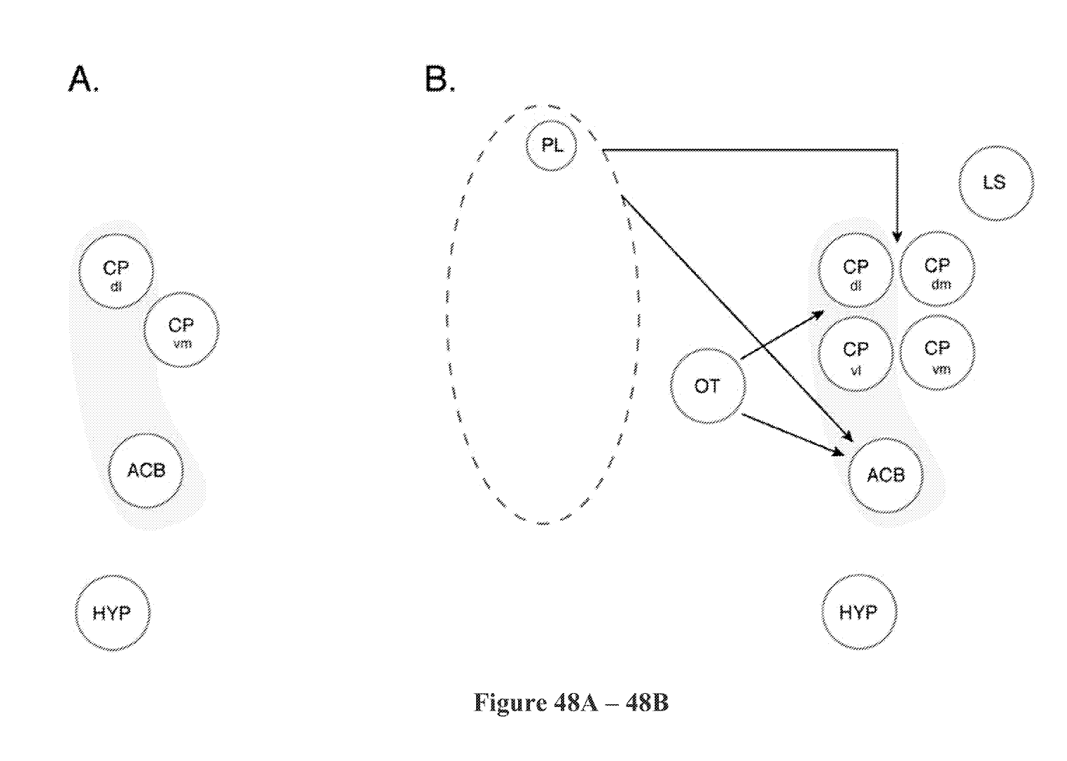

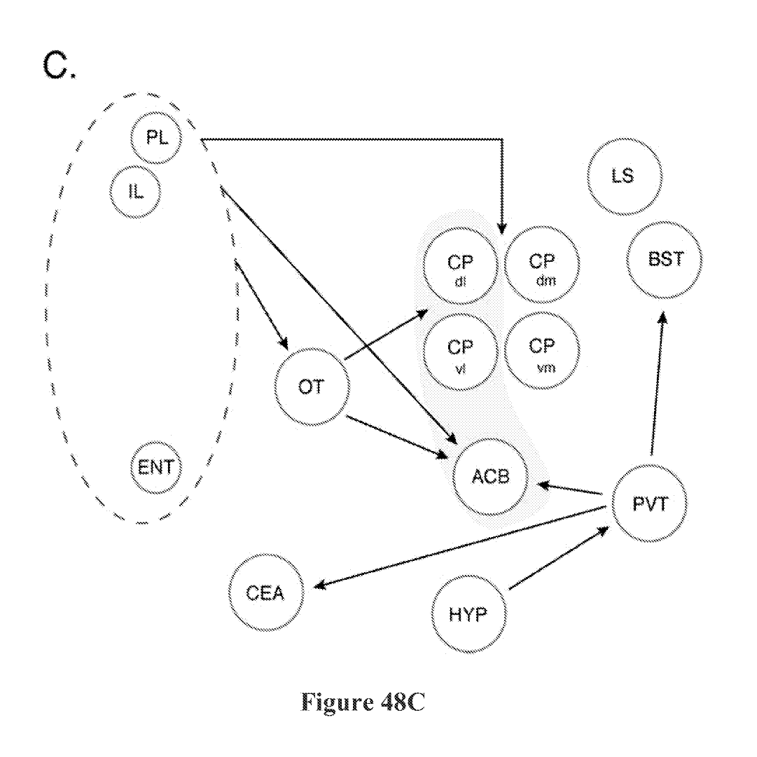

FIG. 5 illustrates data that can comprise pharmacomap data. A pharmacomap represents a multiple dimension (e.g., 3D) distribution of cells in a tissue activated in response to a test compound, as revealed by cellular detection of a reporter product. The pharmacomap data representation may include a multiple dimension (e.g., 3D) dataset. For example, the pharmacomap data representation includes a multiple dimension (e.g., 3D) image and pharmacomap information. The multiple dimension image includes one or more voxels which each includes coordinate data, e.g., x, y, z coordinate data, etc. The pharmacomap information includes information associated with regions, e.g., anatomical segmentation data, etc. A region includes one or more voxels. Additionally, the pharmacomap information includes activated cell data, e.g., the number of activated cells per region, etc. Cells are associated with voxels. As an example, a voxel comprises one or more cells. For further technical details related to a 3D dataset, reference is made to the U.S. Pat. No. 7,724,937, entitled “Systems and methods for volumetric tissue scanning microscopy,” filed May 12, 2008, which is incorporated by reference in its entirety. For further technical details related to a voxel, reference is made to the U.S. Patent Publication No. 2010/0183217, entitled “Method And Apparatus For Image Processing,” filed Apr. 24, 2008, which is incorporated by reference in its entirety. Detailed examples of pharmacomaps of different drugs are shown in FIG. 47 and described in Section 6.9, Example 9. In addition, detailed examples of pharmacomaps of a same drug at different doses are shown in FIG. 48 and described in Section 6.10, Example 10.

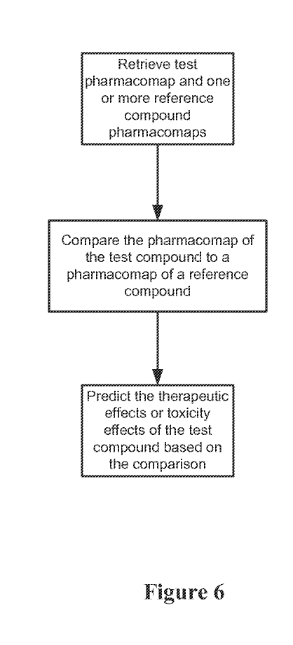

FIG. 6 illustrates operations for analyzing test pharmacomaps with reference pharmacomaps for multiple purposes, such as to identify possible effects of the test compound. One or more reference pharmacomaps may be retrieved from a database of reference pharmacomaps of reference compounds with known effects. A correlation matrix linking the one or more reference pharmacomaps and the known effects of the reference compounds may be generated. For example, if five different drugs show overlapping activation in non-human animal tissue regions X and Y and are known to cause a common therapeutic effect, then it may be predicted that the simultaneous X and Y activation in the tissue represents the common therapeutic effect of these five drugs. Similarly, if two of the five drugs share a therapeutic effect not seen by the other three drugs and show activation in an additional tissue region Z, then it may be predicted that the tissue region Z represents a selective effect of the two drugs.

A test pharmacomap of a test compound may be retrieved from a database of test pharmacomaps. The test pharmacomap may be compared with the one or more reference pharmacomaps. Based on the comparison, therapeutic and/or toxicity effects of the test compound may be predicted. For example, an overlap of activation patterns between the one or more reference pharmacomaps and the test pharmacomap may be used to predict a possible therapeutic effect of the test compound.

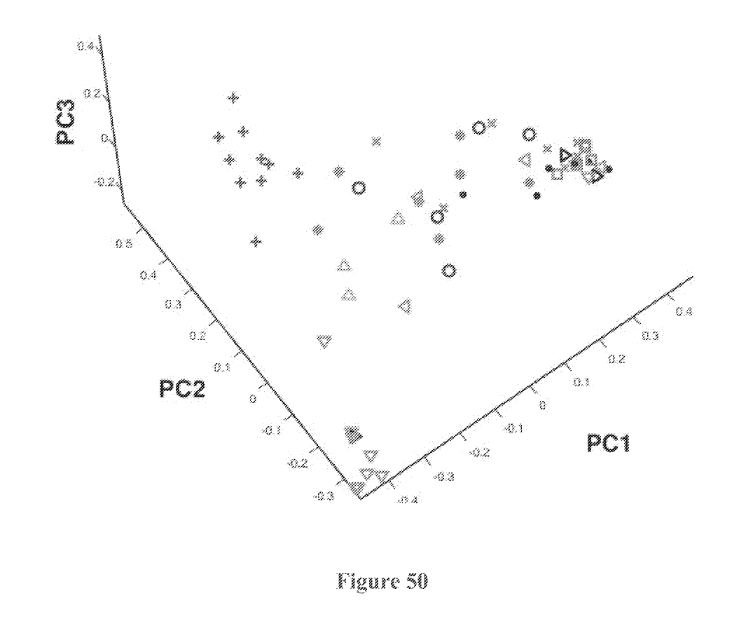

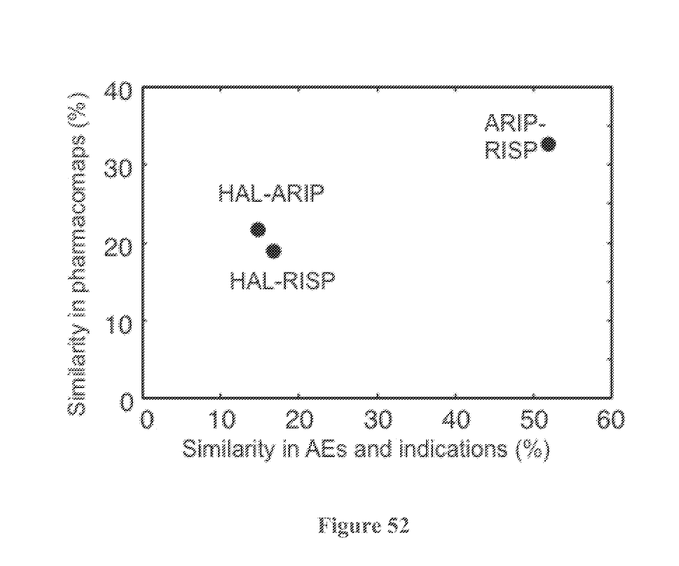

In some embodiments, pharmacomaps can be used to differentiate different drugs, as shown in FIG. 47 and described in Section 6.9, Example 9. In other embodiments, pharmacomaps can be used to differentiate different dosages of a same drug, as shown in FIG. 48 and described in Section 6.10, Example 10. In particular embodiments, pharmacomaps generated from non-human animal issues can be correlated with human clinical outcomes for predicting test compounds' therapeutic effects or adverse effects on humans, as shown in FIGS. 50-52 and described in Section 6.12, Example 12. For example, a pharmacomap of a new drug can be compared to those of known drugs to predict adverse effects and/or indication(s) for the new drug, as shown in FIG. 52.

In some embodiments, the pharmacomaps described herein can be combined with information about structural, physical, and chemical properties (SPCPs) of the tested compounds. In other specific embodiments, the pharmacomaps described herein can be combined with any available information about properties (e.g., side effects) of the tested compounds. For example, the pharmacomaps described herein can be combined with information about properties of the tested compounds available through a database such as Pubchem, BioAssays or ChemBank (which, e.g., may contain information about drug-target interactions and/or cellular phenotypes induced by the drug(s)). In one embodiment, the pharmacomaps described herein can be combined with information about side effects of the tested compounds, e.g., information available through a database such as SIDER. In a particular embodiment, the pharmacomaps described herein can be combined with the data from the SIDER database.

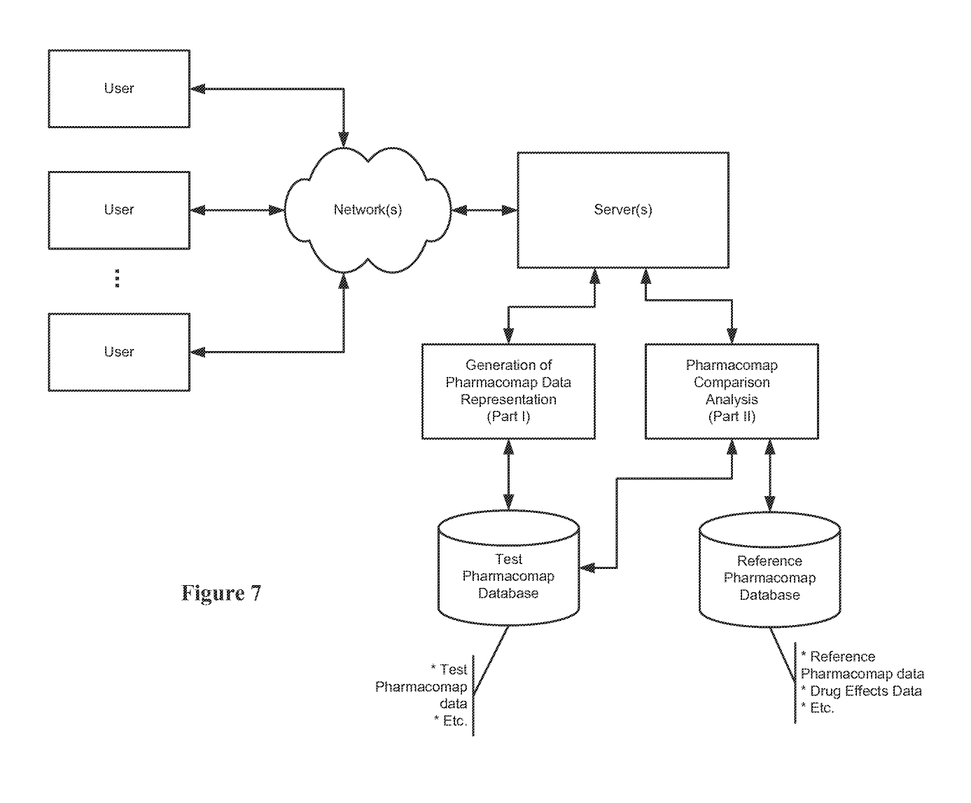

FIG. 7 illustrates an implementation where the test pharmacomap information and the reference pharmacomap are stored in separate databases. Test pharmacomap data representations of test compounds can be generated and stored in a test pharmacomap database. Reference pharmacomap data representations of reference compounds with known effects may be stored in a reference pharmacomap database. For example, the test pharmacomap database may include test pharmacomap data, etc. The reference pharmacomap database may include reference pharmacomap data, drug effects data, toxicity data, etc. The test pharmacomap data representations may be retrieved from the test pharmacomap database to be compared with the reference pharmacomap data representations from the reference pharmacomap database for multiple purposes, e.g., predicting possible effects of the test compounds.

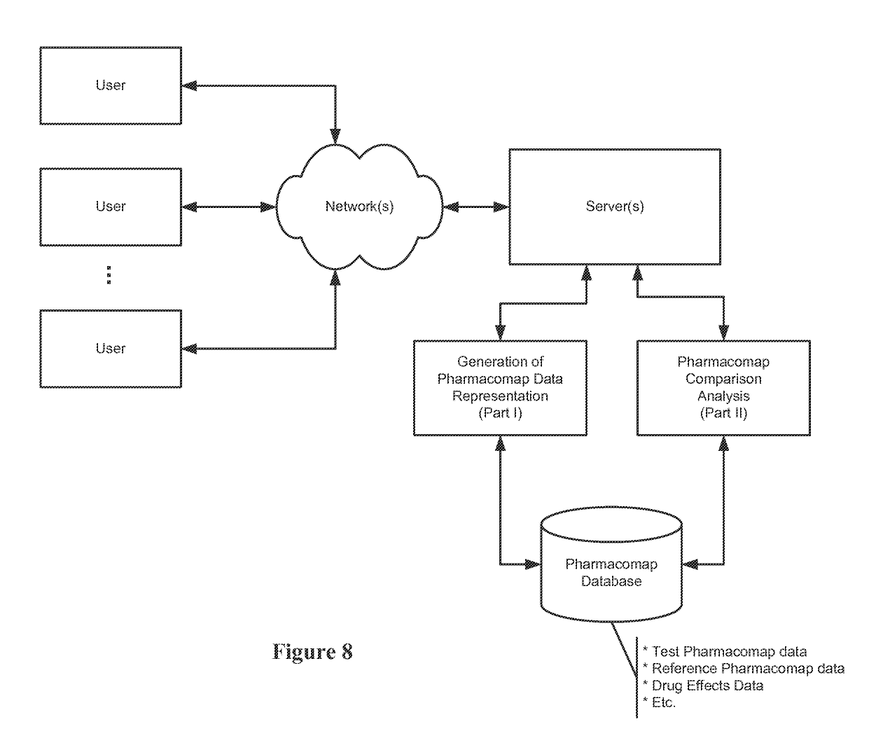

FIG. 8 illustrates an implementation where the test pharmacomap information and the reference pharmacomap are stored in the same database. Test pharmacomap data representations of test compounds and reference pharmacomap data representations of reference compounds may be generated and stored in a same pharmacomap database. For example, the pharmacomap database may include test pharmacomap data, reference pharmacomap data, drug effects data, etc. The test pharmacomap data representations and the reference pharmacomap data representations may be retrieved from the pharmacomap database to be compared for multiple purposes, e.g., predicting possible effects of the test compounds.

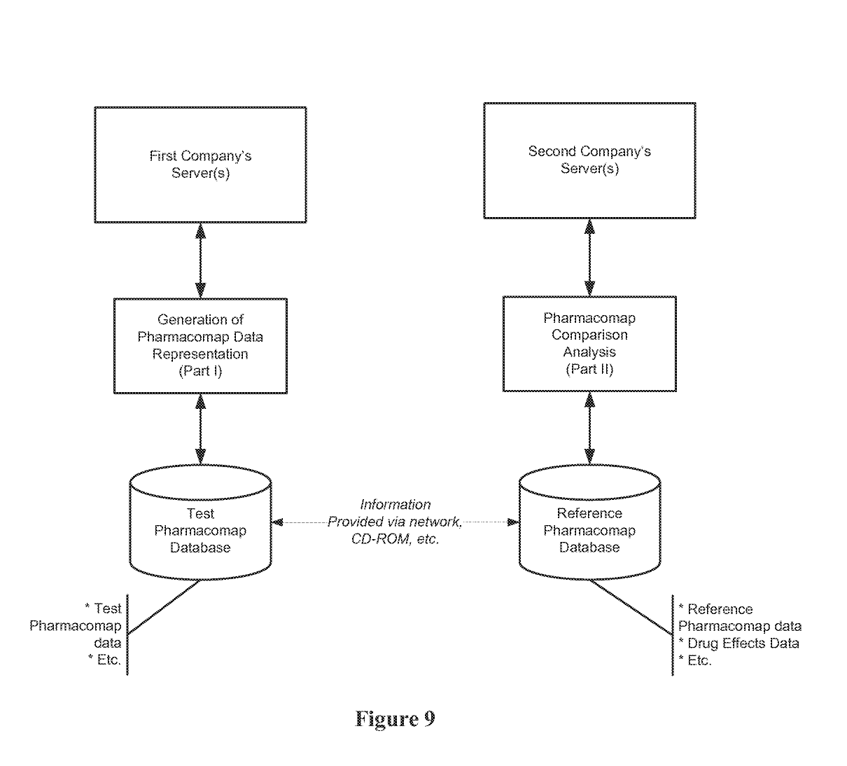

FIG. 9 illustrates an implementation where the test pharmacomap information has been generated and stored by a different company than the company which is to perform the test-reference pharmacomap analysis. Test pharmacomap data representations of test compounds can be generated at a first company's server(s) and stored in a test pharmacomap database. For example, the test pharmacomap database may include test pharmacomap data, etc. Reference pharmacomap data representations of reference compounds can be generated at a second company's server(s) and stored in a reference pharmacomap database. For example, the reference pharmacomap database may include reference pharmacomap data, drug effects data, etc.

Information related to test pharmacomap data representations may be provided, e.g., via a network, CD-ROM, etc., to the reference pharmacomap database for comparison with the reference pharmacomap data representations for multiple purposes, such as to identify possible effects of the test compounds. Similarly, information related to reference pharmacomap data representations may be provided via a network, CD-ROM, etc. to the test pharmacomap database for comparison with the test pharmacomap data representations.

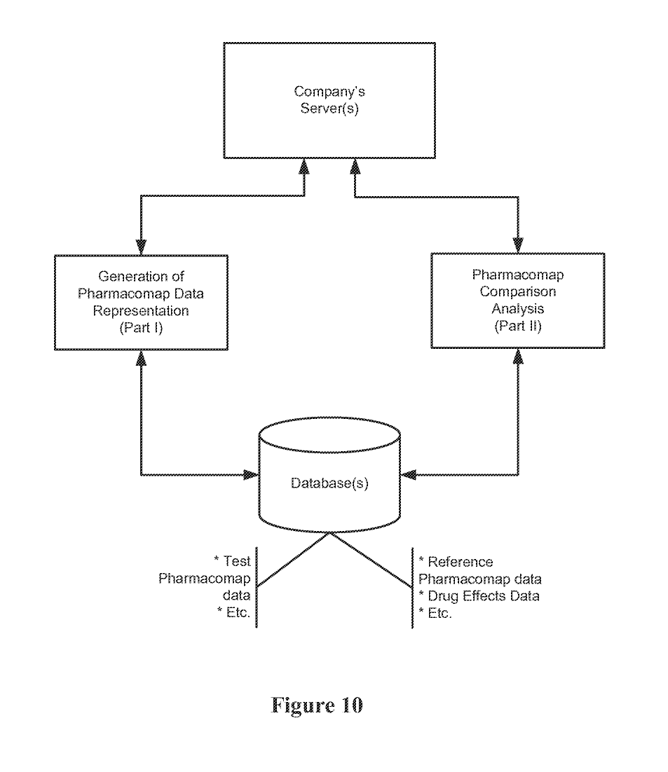

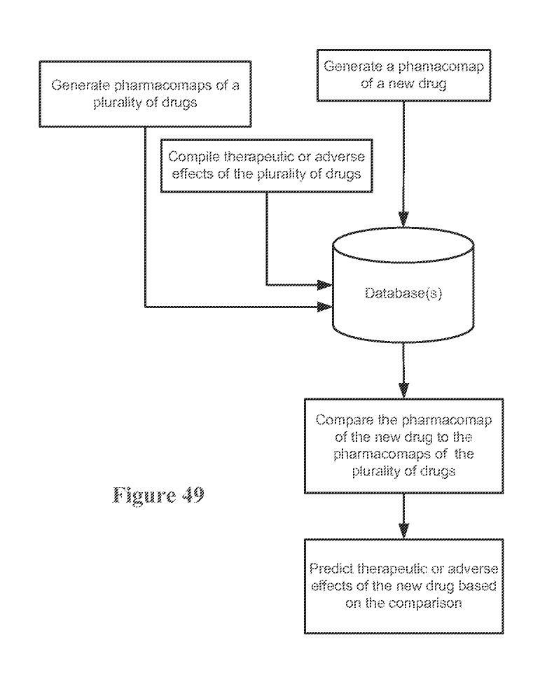

FIG. 10 illustrates an implementation where the test pharmacomap information has been generated and stored by the same company which is to perform the test-reference pharmacomap analysis. Test pharmacomap data representations of test compounds and reference pharmacomap data representations of reference compounds may be generated at a same company's server(s) and stored in a same database. For example, the database may include test pharmacomap data, reference pharmacomap data, drug effects data, etc. Comparison of the test pharmacomap data representations with the reference pharmacomap database may be carried out for multiple purposes, such as to identify possible effects of the test compounds. A more detailed example of generating a comprehensive database of pharmacomaps for predicting therapeutic and adverse effects of new drugs is shown in FIG. 49 and described in Section 6.11, Example 11.

It is further noted that the systems and methods may be implemented on various types of data processor environments (e.g., on one or more data processors) which execute instructions (e.g., software instructions) to perform operations disclosed herein. Non-limiting examples include implementation on a single general purpose computer or workstation, or on a networked system, or in a client-server configuration, or in an application service provider configuration. For example, the methods and systems described herein may be implemented on many different types of processing devices by program code comprising program instructions that are executable by the device processing subsystem. The software program instructions may include source code, object code, machine code, or any other stored data that is operable to cause a processing system to perform the methods and operations described herein. Other implementations may also be used, however, such as firmware or even appropriately designed hardware configured to carry out the methods and systems described herein.

It is further noted that the systems and methods may include data signals conveyed via networks (e.g., local area network, wide area network, internet, combinations thereof, etc.), fiber optic medium, carrier waves, wireless networks, etc. for communication with one or more data processing devices. The data signals can carry any or all of the data disclosed herein that is provided to or from a device.

The systems' and methods' data (e.g., associations, mappings, data input, data output, intermediate data results, final data results, etc.) may be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other computer-readable media for use by a computer program.

The systems and methods may be provided on many different types of computer-readable storage media including computer storage mechanisms (e.g., non-transitory media, such as CD-ROM, diskette, RAM, flash memory, computer's hard drive, etc.) that contain instructions (e.g., software) for use in execution by a processor to perform the methods' operations and implement the systems described herein.

The computer components, software modules, functions, data stores and data structures described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

### 5.6 Other Types of Analysis

In certain embodiments, the tissues of the non-human animals used in accordance with the methods described herein are examined using any approach that allows to determine gene expression (e.g., expression of a native gene or expression of transgene) or to characterize the cells of the tissue in any other way (e.g., morphologically). Such approaches include, without limitation, immunohistochemistry (IHC), biochemical analyses, and in situ hybridization, each of which is well-known in the art. In some of these embodiments, the non-human animals used are transgenic animals. In other embodiments, the non-human animals used are not transgenic animals.

### 6. EXAMPLES

### 6.1 Example 1

### Serial Two-Photon Tomography: An Automated Method for Ex-Vivo Mouse Brain Imaging

In the recent years, the growing focus on systematic generation of complete whole-brain datasets, for example the Allen Mouse Brain Atlas for gene expression (Lein et al., Nature 445, 168-176 (2007)) and the ongoing Mouse Brain Architecture Project for mesoscopic connectivity (Bohland et al., PLoS Computational Biology 5, e1000334 (2009)), has created a pressing need for the development of new instrumentation for high-throughput whole-brain imaging.

This example describes automated high-throughput imaging of fluorescently-labeled whole mouse brains using serial two-photon (STP) tomography which integrates two-photon microscopy and tissue sectioning. STP tomography uses whole-mount two-photon microscopy (Tsai et al., Neuron 39, 27-41 (2003); Ragan et al., Journal of Biomed. Optics 12, 014015 (2007)), and allows generation of datasets of precisely aligned, high-resolution serial optical sections. This example shows that STP tomography generated high-resolution datasets of whole-brain imaging that are free of distortions and that can be readily warped in 3D, for example, for direct comparisons of different whole-brain anatomical tracings.

### Materials and Methods

Tissue Preparation.

The following mouse strains were used: ChAT-GFP Tg(Chat-EGFP) and Mobp-GFP Tg (Gong et al., Nature 425, 917-925 (2003); GFPM (Feng et al., Neuron 28, 41-51 (2000)); SST-ires-Cre::Ai9 (Taniguchi et al., Neuron 71, 995-1013 (2011)); and wild type mice. As anatomical tracers, Cholera toxin B subunit (CTB) Alexa Fluor-488 (0.5% wt/vol in phosphate buffer) and AAV-GFP with synapsin promoter were used (Kugler et al., Virology 311, 89-95 (2003); Dittgen et al., PNAS 101, 18206-18211 (2004)). AAV was produced as a chimeric ½ serotype (Hauck et al., Mol Ther 7, 419-425 (2003)), purified by iodoxinal gradient and concentrated to 5.3×10¹¹ genomic copy per ml. Stereotaxic injections of the tracers were done as described (Cetin et al., Nat. Protocols 1, 3166-3173 (2007)). Briefly, the mice were anaesthetized by 1% isoflurane inhalation. A small craniotomy (approximately 300×300 μm) was opened over the left primary somatosensory cortex and ˜50 nl of virus or 50 nl of 0.05% CTB Alexa Fluor® 488 was injected into layer ⅔ barrel cortex at stereotaxic coordinates: caudal 1.6, lateral 3.2, ventral 0.3 mm relative to bregma. The skin incision was then closed with silk sutures, and the mice were allowed to recover with free access to food and water (meloxicam was given at 1 mg/kg, s.c. for analgesia). The brains were prepared for imaging 10-14 days later (see below).

The mouse brains were prepared for STP tomography as follows. The mice were deeply anesthetized by intraperitoneal (i.p.) injection of the mixture of ketamine (60 mg/kg) and medetomidine (0.5 mg/kg) and transcardially perfused with ˜15 ml cold saline (0.9% NaCl) followed by ˜30 ml cold neutral buffered formaldehyde (NBF, 4% w/v in phosphate buffer, pH 7.4). The brains were dissected out and post-fixed in 4% NBF overnight at 4° C. In order to decrease formaldehyde-induced autofluorescence, the brains were incubated in 0.1 M glycine (adjusted to pH 7. 4 with 1M Tris base) at 4° C. for 2-5 days. The brains were then washed in phosphate buffer (PB) and embedded in 3-5% oxidized agarose as described (Shainoff et al., The Clevelend Clinic Foundation, US, 1982; Sallee & Russell, Biotech Histochem 68, 360-368 (1993)). Briefly, agarose was oxidized by stirring in 10 mM sodium periodate (NaIO₄) solution for 2 hrs at RT, washed 3× and re-suspended in PB to bring the final concentration to 3-5%. The mouse brain was pat-dried and embedded in melted oxidized agarose using a cube-shaped mold. Covalent crosslinking between brain surface and agarose was activated by equilibrating in excess of 0.5-1% sodium borohydrate (NaBH₄) in 0.05 M sodium borate buffer (pH=9.0-9.5), gently shaking for 2-4 hrs at RT (or overnight at 4° C.) (after rinsing, activated agarose can be stored in PB at 4° C. for up to one week; sodium borohydrate buffer should be prepared fresh). Covalent crosslinking of the agar-brain interface is helpful for keeping the brain firmly embedded during sectioning and to limit shadowing artifacts by insufficiently cut meninges.

The Instrument and Software.

The experiments were performed on a high speed multiphoton microscope with integrated vibratome sectioning. Laser light from a titanium sapphire laser was directed through a tube and scan lens assembly towards a pair of galvanometer mirrors and reflected by a short pass dichroic towards a microscope objective (either a 20× lens, NA 1.0, or a 10× lens, NA 0.6). The fluorescent signal from the sample was collected by the same objective, passed through the dichroic and directed by a series of mirrors and lens onto a photomultiplier tube detection system. In two- and three-channel multicolor configuration the emission light was split by dichroic mirror(s) onto, respectively, two and three PMTs to allow for simultaneous multichannel data acquisition. 3D scanning of Z-volume stacks was achieved via a microscope objective piezo, which translates the microscope objective with respect to the sample. Laser light intensity can be varied by liquid crystal controller for shuttering purposes and as a function of imaging depth into the sample.

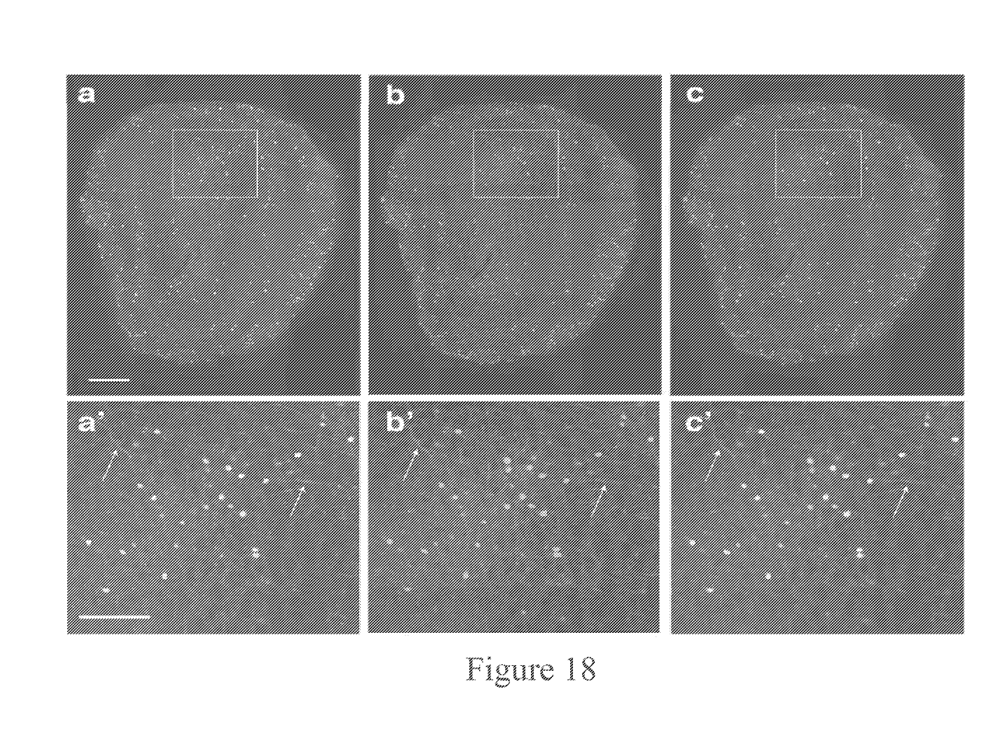

Robust mechanical sectioning was achieved by a vibrating blade microtome that is integrated into the imaging system. It is based on a novel dual flexure design. Flexures are compliant mechanisms consisting of a series of rigid bodes connected by compliant elements that are designed to produce geometrically well defined motion upon application of force. Flexures can achieve smooth displacements down to the sub-micron level with little parasitic motion. The microtome consists of a primary flexure to which the blade is mounted and a secondary flexure which connects the primary flexure to the actuator. The actuator consists of a DC motor with an off-center cam attached to the shaft. The secondary flexure is designed to be rigid in the direction of the cut and compliant in all other directions. In this way, only a force along the direction of the cut is transmitted to the primary flexure which holds the microtome blade and reduces any potential parasitic motion along unwanted axis of motion. For this design, it was experimentally verified that the parasitic Z-vertical deflection was less than 2 μm RMS by measuring the motion directly with capacitive sensors. The vibration frequency can be set between 0-60 Hz and the blade angle between 5-30 degrees. By the use of different cams, the amplitude can be adjusted from 0.8 mm to 2 mm. The sectioning parameters for brain tissue were determined to be 0.8 mm amplitude at 60 Hz and at a blade angle of 11 degrees. The reliability of sectioning was verified by measurements of the brain surface and overlapping Z-planes before and after sectioning during a whole brain dataset (FIG. 18). To achieve reliable sectioning it is important to use brains covalently crosslinked in oxidized agarose.

The instrument was controlled by custom software, written in C++ and C#. It handled the scanning, stage motion, microtome control, and data acquisition. The software was comprised of several discrete services, each of which controlled a particular hardware component or function of the instrument. Sequences of events were coordinated by a master orchestrator service. For instance, in order to scan a section, a command is sent from the orchestrator service to the galvanometer scanner service commanding it to unshutter the laser and scan an image. The orchestrator service waits until the scanner service reports that the image acquisition has been completed, and then sends a command to the XY stage to move the sample to the next position. Once the XY stage completes the requested motion, a command is sent back to the orchestrator service, which in turn issues a command to the scanner service to acquire a second image. During the imaging, background services handled the data acquisition and saving of the 16 bit TIFF images to a local or network attached storage device. The process continued until an entire section had been acquired. Similarly, to acquire a whole-brain dataset, at the end of each mosaic section acquisition the orchestrator service commanded the Z-stage service to move the sample upwards by the desired slice thickness. Simultaneously, the sample was directed towards the microtome by the XY stage service. Once in position, the microtome was turned on and the sample was translated through the microtome and a tissue section was cut. The sample was then translated back underneath the objective, and the next section was imaged. This process was repeated until all sections were imaged. The software is highly modular and additional services can be introduced or specific hardware can be exchanged with minimal changes to higher level routines. For instance, services to automate additional features, such as the capture of the slices after sectioning, can be added in the future.

In comparison to an earlier prototype (Ragan et al., Journal of Biomed. Optics 12, 014015 (2007)) there are a significant number of improvements in the design of the instrument used in this example. The previous version used a milling machine to machine the surface of a paraffin embedded tissue. Because paraffin quenches fluorescence, an integrated vibrating blade microtome was used in this example. This allows imaging of formaldehyde fixed brains embedded in agar, a histological procedure with low quenching. As an additional advantage, the sections can be used for further histochemical analysis as they are no longer destroyed by the milling process (the sections sink to the bottom of the water bath and can be collected and sorted at the end of the experiment). The incorporation of low-magnification (10-20×) high-numerical aperture (NA 0.6-1.0) lenses has increased fluorescence collection compared to a standard 60× objective, without compromising the resolution at large imaging depths (Oheim et al., Journal of Neuroscience Methods 111, 29-37 (2001)). The combination of a low-magnification lens with large aperture optics have increased the image field of view that can be scanned with even illumination from ˜200 to 1400 μm. High speed galvanometric scanning has replaced a polygonal scanning approach. Galvanometric scanners are far more flexible than polygonal scanners and allow a wide range of pixel sizes and residence times to be set depending on the requirements of the sample. Finally, a high speed custom XYZ stage was constructed to allow positioning of the sample over centimeters of travel with sub-micron accuracy. The custom Z-stage was designed to hold two commercial X and Y stages and be rotationally rigid with a pitch and yaw of less than 1 micron over the entire travel range of the X and Y stage assembly. The X and Y axes have a 0.1 μm positional accuracy, a settling time of 0.1 ms and a speed up to 50 mm/s. The high speed and small settling time allows for rapid positioning of the sample and minimizes acquisition time of a section, while the positional accuracy decreases post-processing registration time. The Z-axis has a precision of 0.15 μm and a maximum velocity of 1 mm/s Since this stage was only used to raise the sample to the microtome blade and objective, its speed had negligible impact on the imaging time.

The Instrument Operation.

Once the brain was positioned under the objective and the imaging and sectioning parameters were chosen (see below), the instrument operated in a fully automated mode. The brain was mounted in saline (50 mM PB, pH 7.4) in a water bath positioned on the computer controlled XYZ stage. After identifying Z-position of the brain surface under the objective, the following parameters were set in the software: FOV size, FOV mosaic size, pixel size, pixel residence time, laser power, sectioning speed, sectioning frequency, Z-step for each sectioning cycle and a number of Z sections. The imaging plane was set below the brain surface to ensure an undisturbed optical section throughout. Typically 50 μm below surface is used, but a comparable image resolution can be obtained down to about 100 μm below surface with small adjustments in laser power. The laser power was set constant for imaging of single optical sections between each sectioning steps. For collection of Z-volumes between sectioning steps, such as the dataset of SST-ires-Cre::Ai9 olfactory bulb imaged at Z-resolution 2.5 μm, the laser power was adjusted based on the Z depth to compensate for increased light scattering with increased depth.

The number of FOV tiles per mosaic was set to cover the extent of the sample and allow for a small overlap between the FOV tiles for post-processing stitching (see below). The experiments with the 10× objective employed 6×8 overlapping mosaic of 1.66×1.66 mm FOV, the XY stage movement is 1.5 mm, pixel size 1 or 2 μm and pixel residence time between 0.4 to 1.0 μs. The experiments with the 20× objective employed 11×17 mosaic of 0.83×0.83 mm FOV, the XY stage movement is 0.7 mm, pixel size 0.5 or 1 μm and pixel residence time between 0.4 to 1 μs. Once a mosaic is completed, the same XYZ stage used for the mosaic imaging moves the sample from the microscope objective towards a vibrating blade microtome to section the uppermost portion of the tissue. The times for imaging of 260 section mouse brain datasets are given in Table 1.

TABLE 1
Imaging conditions for STP tomography

| | | | | | | | Time per |
|---|---|---|---|---|---|---|---|
| | | | Sampling | | Pixel | Time per | 260 |
| objective/ | FOV | FOV | rate x-y | Mosaic | residence | 1 section | sections |
| NA | (mm) | (pixels) | (μm) | of FOVs | time (μs) | (min:sec) | (hrs:min) |
| 10x/0.6 | 1.66 × 1.66 | 832 × 832 | 2.0 | 6 × 8 | 0.8 | 1:30 | 6:30 |
| 10x/0.6 | 1.66 × 1.66 | 1664 × 1664 | 1.0 | 6 × 8 | 0.4 | 2:00 | 8:40 |
| 20x/1.0 | 0.83 × 0.83 | 832 × 832 | 1.0 | 11 × 17 | 0.8 | 3:35 | 15:30 |
| 20x/1.0 | 0.83 × 0.83 | 1664 × 1664 | 0.5 | 11 × 17 | 0.4 | 5:35 | 24:10 |

The time per 1 section and time per 260 sections correspond to imaging conditions with the 10x and 20 objectives, number of FOVs, sampling XY rate and pixel residence time as indicated. The time per 1 section comprises: 1) imaging time, 2) mosaicing movement of XY stages, and 3) sectioning time. Imaging time comprises most of the total time and varies based on sampling resolution and pixel residence time. The XY stage movement is about ~0.3 sec per move (~15 sec for 6 × 8 mosaic and ~1 min for 11 × 17 mosaic). The sectioning time, at stage movement of 1 mm per sec, is ~35 second per cycle.

Image Processing.

The images were constructed from the PMT signal, with the tile and pixel size set by a combination of the scan angle and pixel sampling rate. The tiles were saved as tif files (named as Tile_Z{zzz}_Y{yyy}_X{xxx}.tif) and processed in the following way. First, each tile was cropped to remove illumination artifacts near the edges (the number of pixels cropped is determined empirically based on the objective used and FOV; e.g. 15 and 10 pixels were cropped at each side of X and Y direction, respectively, for 832×832 pixel FOV). Second, all tiles from one brain dataset (for example 52,360 tiles for 11×17 mosaic of 280 sections) were loaded in Fiji ImageJ-based image-processing software, and used to generate an average-intensity image for illumination correction by a Z-project function. Third, all tiles were divided by the average-intensity image to correct for uneven illumination (Plugins>TissueVision>Divide sequence by image). Fourth, illumination-corrected tiles were used to stitch the sequence of mosaic images (Plugins>Stitching>Stitch Sequence of Grids of Images; fusion method=linear blending, fusion alpha=1.5, regression threshold=0.3, max/avg displacement=2.5, absolute displacement=3.5; select “compute overlap”). The transformation between the tiles was modeled as a translation transform. For each section, the X and Y translations were determined by cross correlation (Kuo et al, Proceedings of the Optical Society of America Meeting on Understanding and Machine Vision 7376 (1989)) between the tiles. At the overlapping regions, the pixels were blended linearly (Preibisch et al., Bioinformatics 25, 1463-1465 (2009); Cardona et al., The Journal of Neuroscience 30, 7538-7553 (2010)). The overlapping regions may show some photobleaching when large power (>150 mW) is used for samples with low fluorescence. In such case, since bleaching occurs mainly for the second overlapping tile, it is better to display the image from the first tile and use the second tile only for XY registration. This can be achieved by rendering the tiles into the mosaic in the reverse order they where scanned by the microscope: the pixels of the first scanned tile overwrite the same pixels scanned later in the second. The whole brain dataset of 11×17 mosaic of 280 sections of raw tiles was scanned at a 16-bit depth occupies ˜40 GB. The final stitched slices occupied ˜25 GB with LZW compression on the final stitched TIFF slices. All image processing was run on Mac/Linux desktop machines with at least 8 GB on RAM.

Image Warping.

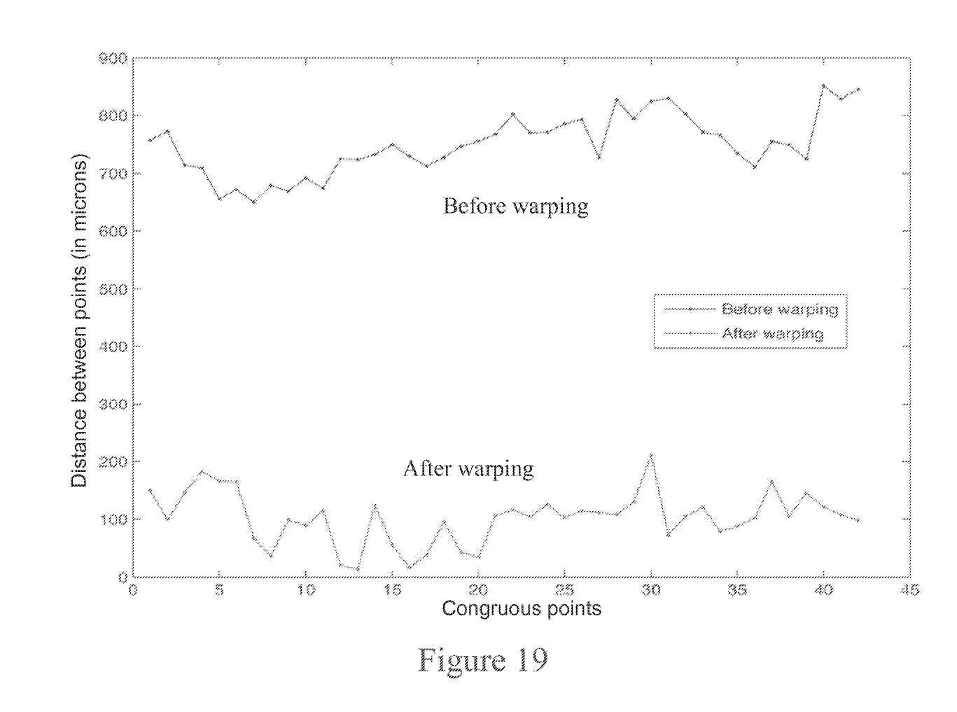

The warping was done by an affine registration followed by an elastic B-spline-based transformation (Klein et al., IEEE Transactions on Medical Imaging 29, 196-205 (2010)) using autofluorescence signal from STP tomography datasets downsampled by factor of 20 (resolution 20×20×50 μm). The registration was done in a multi-resolution approach for a more efficient and robust alignment (Lester et al., Pattern Recognition 32, 129-149 (1999)). The affine transform was calculated using 4 resolution levels while the elastic step uses 6 resolution steps. Advanced Mattes mutual information (Mattes et al., IEEE Transactions in Medical Imaging 22, 120-128 (2003)) was used as the metric to measure the similarity of registration. In this parametric registration method, Mattes Mutual information is used as the similarity measure between the moving and fixed images. The registration problem is posed as an optimization problem, where the image discrepancy/similarity function is minimized for a set of transformation parameters. The transformation parameters are then estimated in multi-resolution approach, which ensures a more robust approach compared to a single resolution approach. The image similarity function is estimated and minimized for a set of randomly chosen samples with the images at each resolution in a iterative way. On a 8 core CPU with 16 GB RAM, the registration takes 12 hrs on 650×450×300 sized image with 20×20×50 micron pixel spacing. The entire image warping experiment was setup using elastix (Klein et al., IEEE Transactions on Medical Imaging 29, 196-205 (2010)), an image registration tool based on Kitware's ITK with Parameters setup according to used dataset. To determine the effectiveness of the warping procedure, the displacement of 42 anatomical manually identified landmark points of interest was compared in two mouse brain scans before and after warping one dataset onto the other (FIG. 19). The mean (±SEM) distance between the corresponding points in the two brains was 749.5±52.1 and 102.5±45 μm before and after warping, respectively (in FIG. 19, the line above is before warping; the line below is after warping).

### Experimental Design and Results

The versatility of STP tomography was tested by imaging four mouse brains with cell type-specific fluorescent protein expression and systematically mapping input and output connections of mouse somatosensory cortex. These experiments showed that STP tomography is a robust imaging method that can transform the emerging field of systematic whole-brain anatomy, until now limited to dedicated atlasing initiatives (Lein et al., Nature 445, 168-176 (2007); Bohland et al., PLoS Computational Biology 5, e1000334 (2009)), into a routine methodology applicable, for example, to the study of mouse models of human brain disorders in standard laboratory settings.

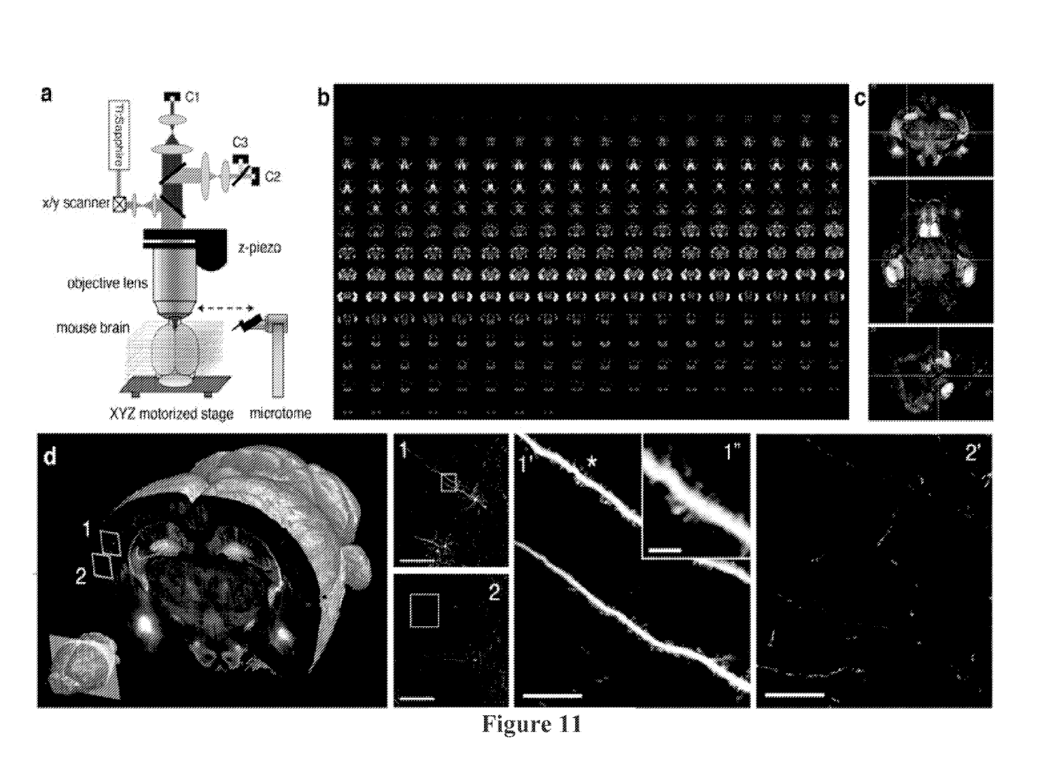

STP tomography works as described below and depicted in FIG. 11. First, a fixed agar-embedded mouse brain is placed in a water bath on XYZ stage under the objective of a two-photon microscope (Denk et al., Science 248, 73-76 (1990)) and imaging parameters are entered in the operating software (see Materials and Methods, supra). Once the parameters are set, the instrument works fully automatically: 1) the XYZ stage moves the brain under the objective so that an optical section (or an optical Z-stack) is imaged as a mosaic of fields of view (FOVs), 2) a built-in vibrating blade microtome mechanically cuts off a tissue section from the top, and 3) the steps of overlapping optical and mechanical sectioning are repeated until the whole dataset is collected. The instrument is a modification of a previous prototype (Ragan et al., Journal of Biomedical Optics 12, 014015 (2007)), that was redesigned for imaging of fluorescently labeled mouse brains, including the integration of a custom-build vibrating blade microtome instead of a milling machine and the use of high-speed galvanometric scanners instead of a rotating polygonal scanner (see Materials and Methods, supra). Sectioning by vibrating blade microtome allows the use of brains prepared by simple procedures of formaldehyde fixation and agar embedding, which have minimal detrimental effects on fluorescence and brain morphology. High-speed galvanometric scanning enables fast imaging and switching between different sampling resolutions for different experiments (see below).

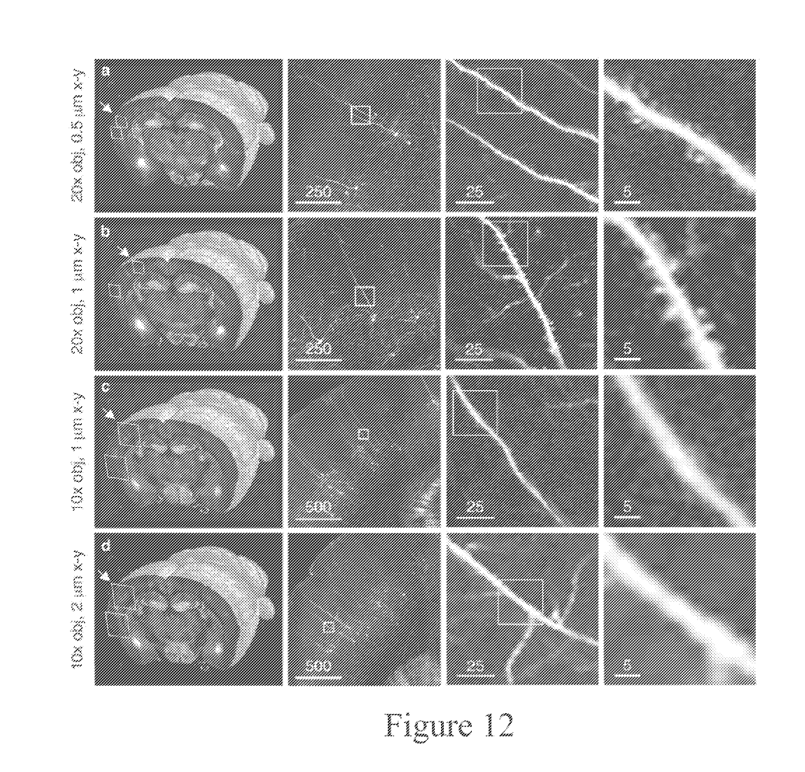

In the first set of experiments, Thy1-GFPM mice (Feng et al., Neuron 28, 41-51 (2000)), which express green fluorescent protein (GFP) mainly in hippocampal and cortical pyramidal neurons, was used to determine the optimal conditions for imaging mouse brains at different sampling resolutions. The GFPM brain was imaged as a dataset of 260 coronal sections, evenly spaced by 50 μm, with 10× and 20× objectives at XY imaging resolution 2.0, 1.0 and 0.5 μm (FIGS. 11 and 12). The 10× objective (0.6 NA) allowed fast imaging at a resolution sufficient to visualize the distribution and morphology of GFP-labeled neurons, including their dendrites and axons (FIG. 12). The data collection times for a 10×-objective dataset of 260 coronal sections were ˜6½ and 8½ hrs at x-y sampling of 2 and 1 μm, respectively (Table 1). The 20× objective (1.0 NA) enabled visualization of dendritic spines and fine axonal arborizations (FIGS. 11 and 12); note that in this application axons are detected within single XY optical sections, but not traced in Z, because of the spacing of 50 μm between each section). The data collection times for a 260-section dataset using the 20× objective were ˜15½ and 24 hrs at x-y sampling of 1 and 0.5 μm, respectively (Table 1). Taken together, these experiments showed that STP tomography can be used as an automated high-throughput method for collection of high resolution datasets of fixed, fluorescently labeled mouse brains.

Transgenic mice with cell type-specific fluorescent protein expression allow easy identification of different types of neurons and glia. In the second set of experiments, whole-brain mapping of different cell types was performed in two BAC transgenic mice and one gene-targeted (knockin) mouse. The Mobp-GFP (Gong et al., Nature 425, 917-925 (2003)) mouse revealed a pattern of whole-brain myelination as a result of GFP expression in oligodendrocytes from the promoter of myelin-associated oligodendrocyte basic protein (Mobp). The ChAT-GFP mouse allowed visualization of whole-brain cholinergic innervation as a result of GFP expression in cholinergic neurons from the choline acetyltransferase (ChAT) promoter. The SST-ires-Cre::Ai9 (Taniguchi et al., Neuron 71, 995-1013 (2011)) mouse revealed brain-wide distribution of somatostatin-expressing interneurons as a result of Cre recombinase expression from the somatostatin (SST) gene, which activates the Ai9 tdTomato-based reporter (Madisen, L., et al., Nature neuroscience 13, 133-140 (2010)). These experiments showed the ease of generating brain atlas-like datasets of cell-type distribution and innervation by STP tomography of GFP-expressing transgenic mice. In addition, a more complete visualization of a specific cell-type distribution can be achieved by imaging Z-stack volumes, instead of single optical sections, between the steps of mechanical tissue sectioning. As an example of this application, described is a dataset of 800 optical sections (2.5 μm Z-spacing) revealing the distribution of all somatostatin-expressing interneurons in the olfactory bulbs of the SST-ires-Cre::Ai9 mouse. Imaging with high Z resolution, of course, increases the acquisition time and currently it would take about 7 days to image a whole mouse brain at the same resolution. However, increasing the imaging speed (at present 0.4 μs pixel residence time) by, for example, integration of resonant scanners (Wilt et al., Annual review of neuroscience 32, 435-506 (2009)), should make high Z-resolution imaging of whole mouse brains by STP tomography more practical in the future.

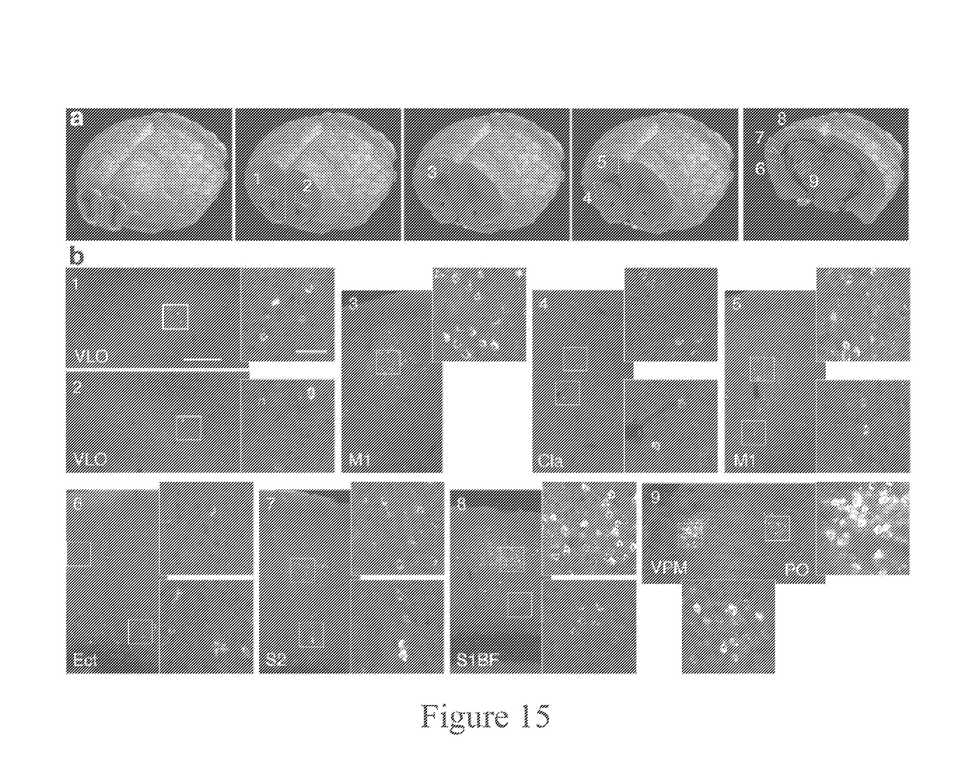

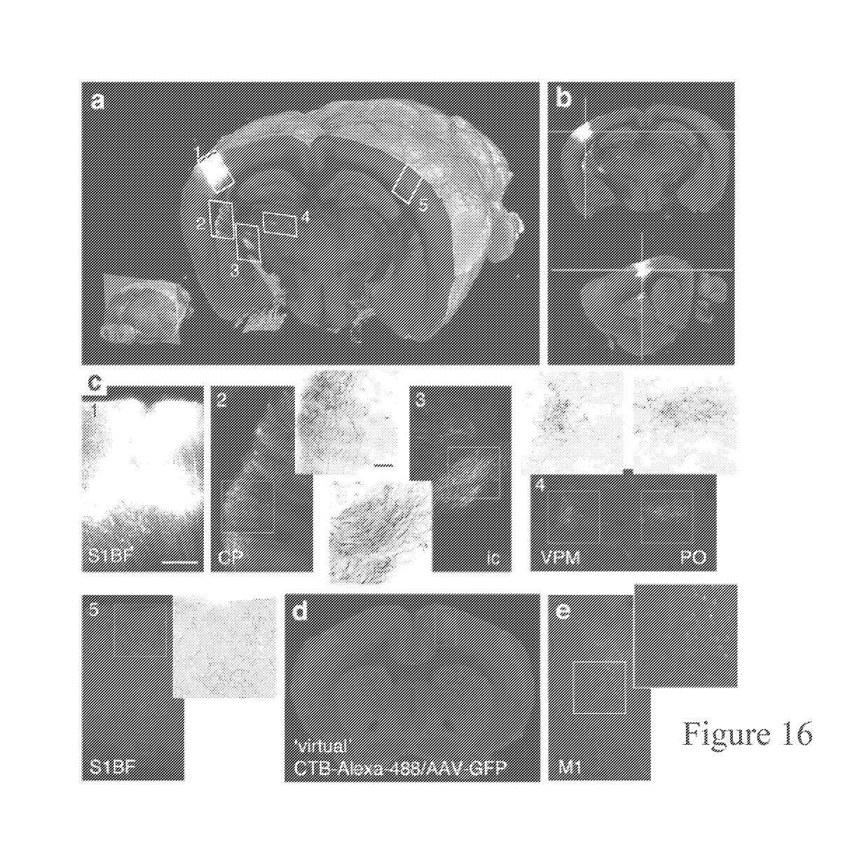

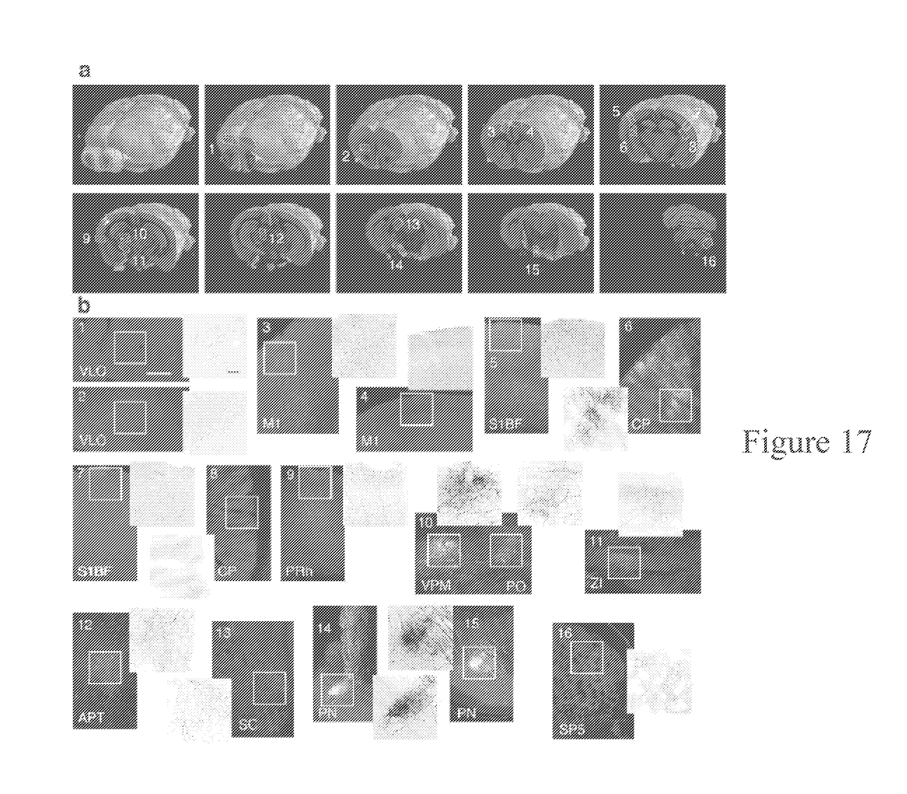

In the final set of experiments, the use of STP tomography for mapping brain connectivity was demonstrated by imaging mouse brains injected with anatomical tracers in the somatosensory barrel cortex, a brain region with projections well documented by both retro- and anterograde tracers (Aronoff et al., The European journal of neuroscience 31, 2221-2233 (2010); Welker et al., Experimental brain research. Experimentelle Hirnforschung 73, 411-435 (1988); Hoffer et al., The Journal of comparative neurology 488, 82-100 (2005)). Brains injected with CTB-Alexa-488 were imaged for retrograde tracing and adeno-associated virus expressing GFP (AAV-GFP) for anterograde tracing at 1 μm XY resolution (20× objective). As expected, Alexa-488-labeled neurons were found in brain areas known to project to the mouse barrel cortex (Aronoff et al. 2010; Welker et al. 1988; Hoffer et al. 2005), and GFP-labeled axons were detected in brain areas known to receive barrel cortex projections (Aronoff et al. 2010; Welker et al. 1988) (FIGS. 14-17). The experiments also revealed two brain regions with sparse connectivity that were not previously reported in the literature: retrogradely labeled contralateral orbital cortex (FIG. 15b, panel 2) and anterogradely labeled contralateral motor cortex (FIG. 17b, panel 2). Taken together, the replication of the previously described pattern of connectivity and the detection of putative new connections in the contralateral cortical areas demonstrate that STP tomography is both a high-throughput and highly sensitive imaging method for anatomical tracing in the whole mouse brain. The 3D alignment of the datasets, in addition, facilitates direct comparison between different samples. This was demonstrated by warping the AAV-GFP brain onto the CTB Alexa-488 brain for direct comparison of anterograde and retrograde tracings (FIG. 16; see Materials and Methods, supra). The precision of co-registration of anatomical landmarks between two brains was estimated to be approximately 100 μm (FIG. 18). Warping of multiple brains to one space thus provides a simple alternative to multiple tracer injections and can be extended to include many brains in a virtual brainbow-like tracing (Livet et al., Nature 450, 56-62 (2007)).

In summary, this example shows that STP tomography can be used to generate high-resolution anatomical datasets that can be readily warped for comparison of multiple brains. STP tomography can be used for systematic studies of brain anatomy in genetic mouse models of cognitive disorders, such as autism and schizophrenia. To provide quantitative measurements for such studies, the focus is being made on anatomical registration (Hawrylycz et al., PLoS computational biology 7, e1001065 (2011)), and the development of computational methods for detection of fluorescence signals in whole-brain datasets generated by STP tomography.

### 6.2 Example 2

### Quantitative Mapping of Neural Circuits in the Mouse Brain Using Serial Two-Photon Tomography

This example describes use of serial Two-Photon (STP) tomography, combining two-photon imaging with a build-in vibratome, for quantitative, fast, ex-vivo 3D mapping of neural circuits in the whole mouse brain.

In this example, stereotaxic delivery (Cetin et al., 2007) of anterograde (AAV) or retrograde (CTB-AF and latex microspheres) fluorescent neuronal tracers was used for output and input projection labeling. After 3D image reconstruction, the standard brain atlas was warped onto the sample brain volume to delineate brain areas of interest and the number of cells per area was counted. The quantitative map of the retrogradely and anterogradely labeled neurons in the whole mouse brain was generated, and the distribution of the fluorescent neurons for different tracer types was compared.

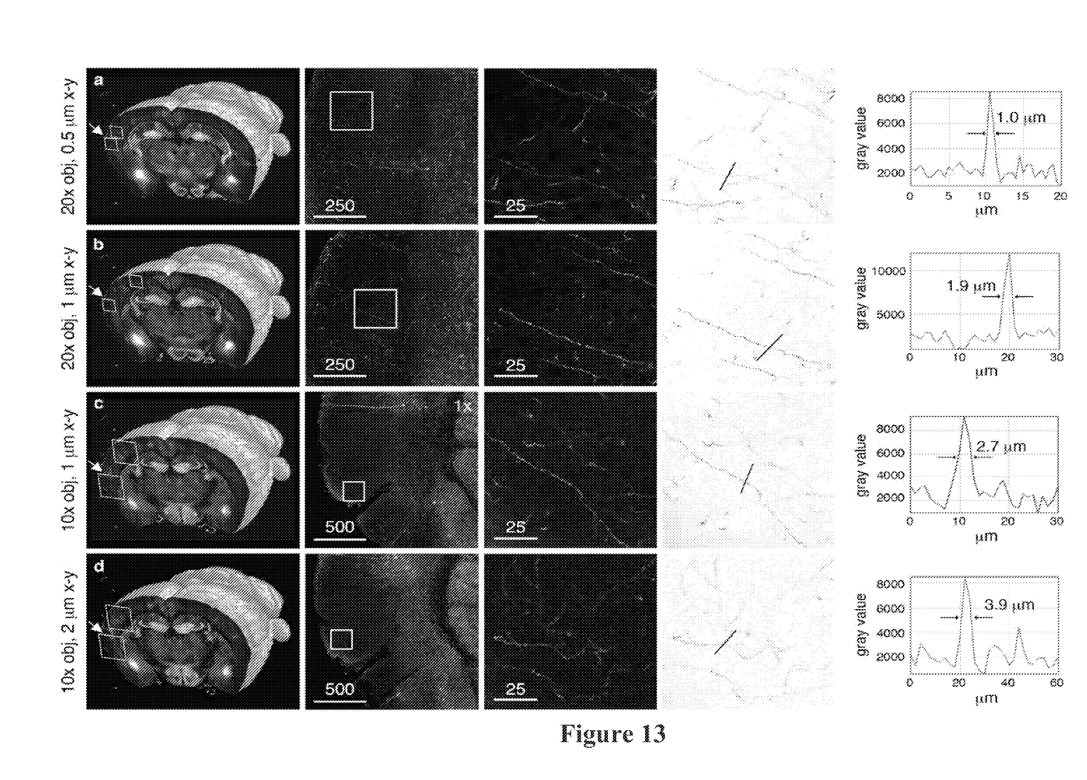

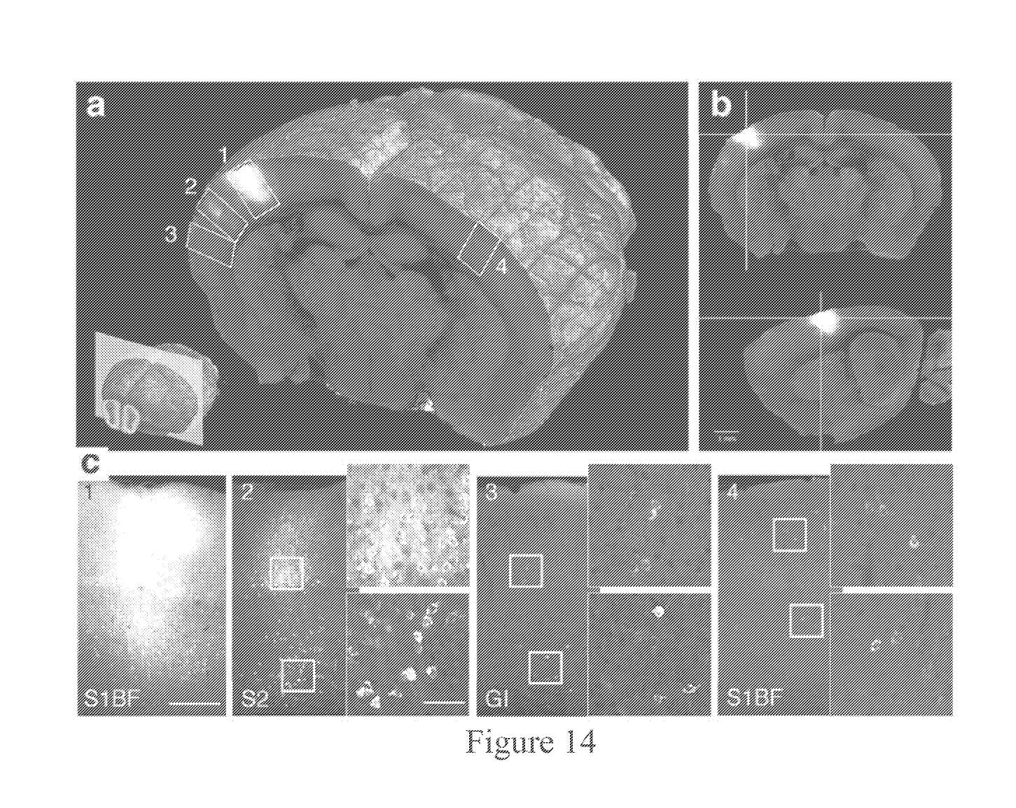

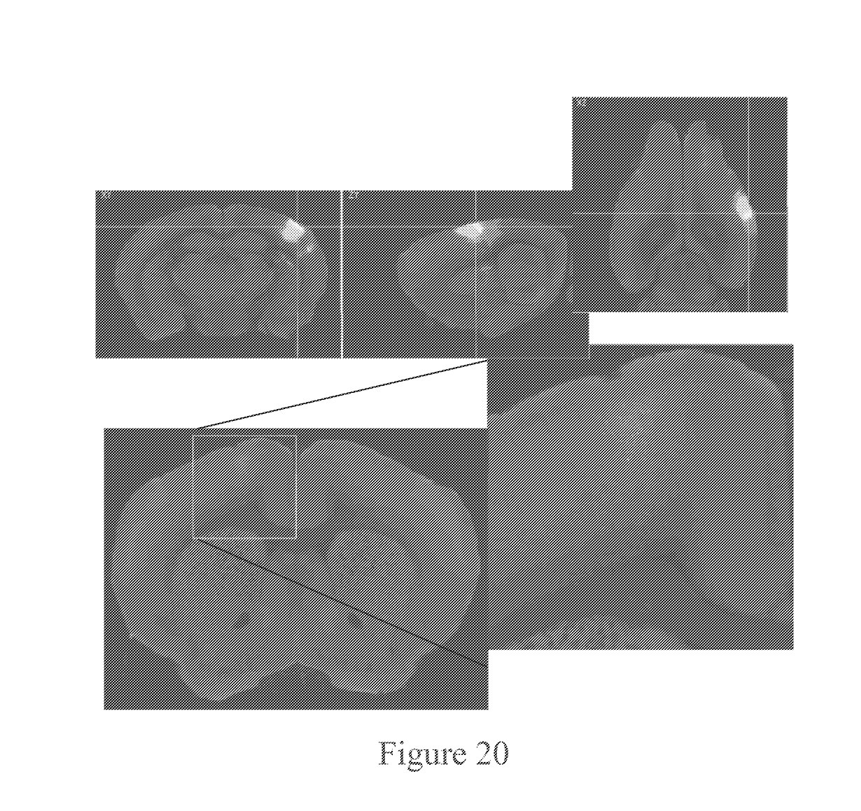

STP tomography imaging, and retrograde/anterograde tracing was performed as described in Example 1 and shown in FIGS. 11-17 and Table 1. FIG. 20 shows combined “virtual” two-tracer dataset generated by warping AAV-GFP brain onto CTB-Alexa-488 brain.

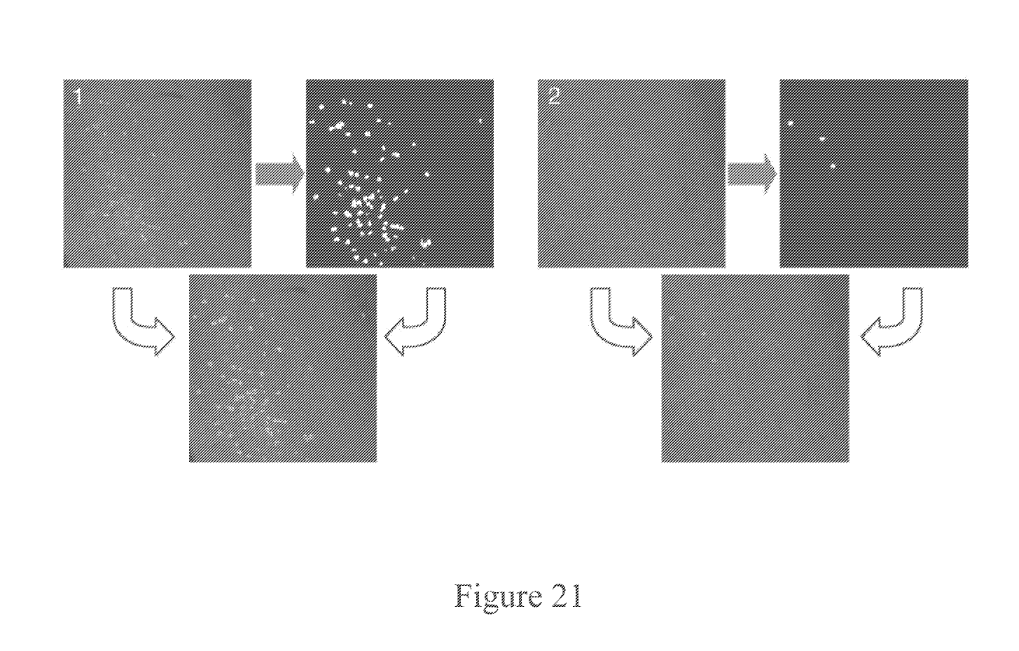

Next, computation detection of CTB-Alexa was performed. Machine learning algorithms were trained to detect CTB-Alexa-488 labeling based on initial human markups and detect CTB-positive cells automatically. FIG. 21 shows exemplary images of before (left panel) and after (right panel) prediction, and overlays of such images (lower panels).

This example demonstrates that STP tomography is a method that can be used for fully automated high-resolution imaging of fluorescently labeled mouse brains. Test brains of retrograde and anterograde tracings revealed regions previously described as well as sparsely labeled regions not reported before, i.e., retrograde contralateral VLO and anterograde contralateral M1. This example also shows that warping of multiple brain samples onto each other can be used to create virtual “brainbow-like” datasets. Fourth, this example shows that computational detection by machine learning algorithms can be used to automate analysis of anterograde and/or retrograde tracing in the whole brain.

### 6.3 Example 3

### Mapping c-Fos-GFP Expression in the Transgenic c-Fos-GFP Mouse Brain Using Automated Imaging and Data Analysis Pipeline

This example demonstrates application of the whole-mount microscopy and the data analysis pipeline for mapping c-fos-GFP expression in the transgenic c-fos-GFP mouse brain.

Making of Transgenic c-Fos Indicator Mice.

High-throughput whole-brain imaging of an immediate early gene (IEG) induction was used in transgenic “indicator” mice that express GFP from specific IEG promoters, such as c-fos and Arc promoters in c-fos-GFP and Arc-GFP transgenic mice ((Barth et al., J Neurosci 24, 6466-6475 (2004); Grinevich et al., Journal of Neuroscience Methods 184, 25-36 (2009)). In these mice, GFP represents a readily detectable surrogate for the expression of the native gene.

Microscopy.

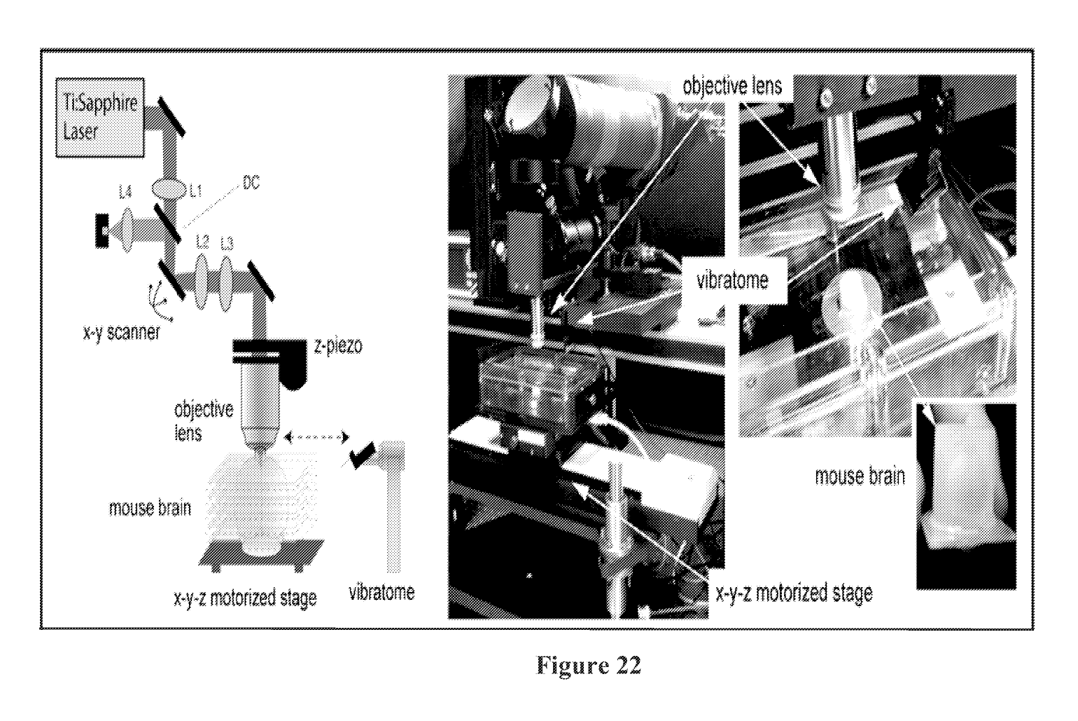

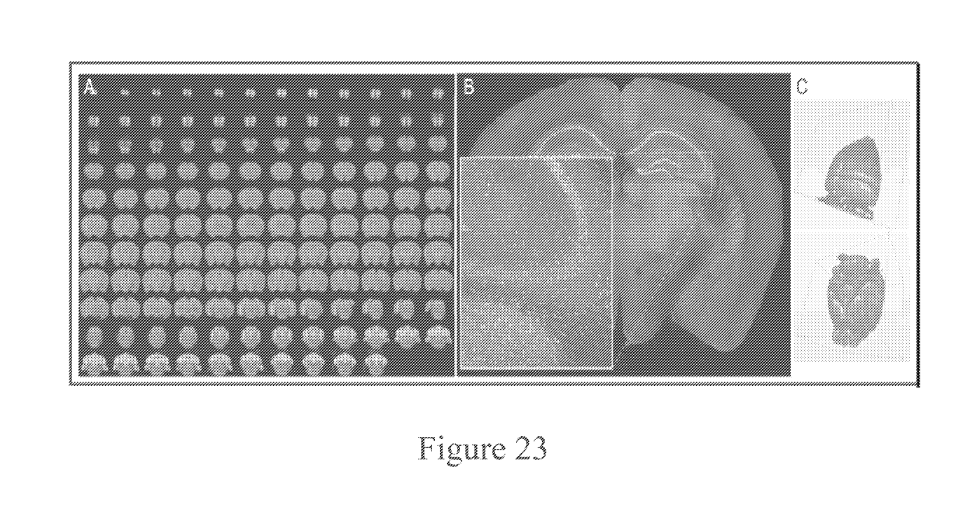

Whole-mount two-photon microscopy was used for automated mouse brain imaging. The instrument works as follows: First, a fixed mouse brain embedded in an agar block is placed in a water bath on top of a computer controlled x-y-z stage. The stage moves the brain under the objective, so that the top is imaged as a mosaic of individual fields of view (“tiles”). Next, a built-in vibratome cuts off the imaged top region, and the cycles of imaging and sectioning repeat until the whole dataset is collected (FIGS. 22 and 23).

Brain Morphing.

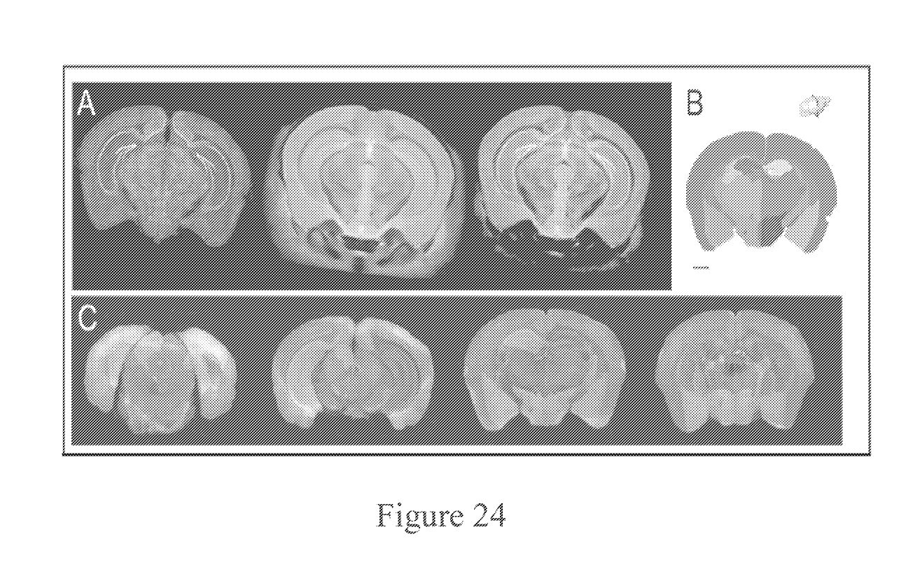

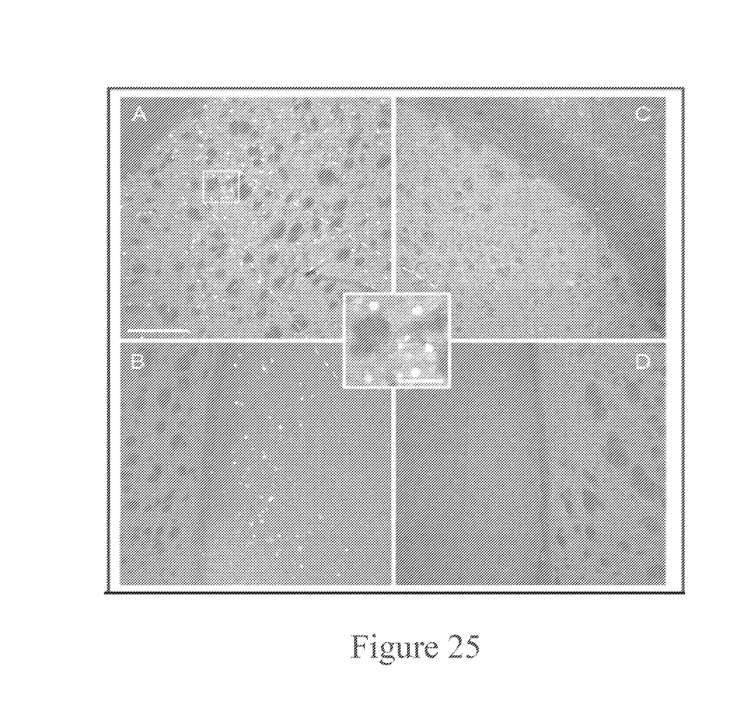

The imaged brain sections were next morphed to a mouse brain atlas generated by high-resolution magnetic resonance imaging (MRI) (Dorr et al., NeuroImage 42, 60-69 (2008)) (FIG. 24). This provided gross anatomical registration within a template X-Y-Z volume that is used for voxelization-based statistical comparisons between samples, as described below.

Computational Detection of c-Fos-GFP.

The imaging conditions for the transgenic c-fos-GFP brains were previously optimized, using a systemic delivery of the antipsychotic drug haloperidol (Dragunow et al., Neuroscience 37, 287-294 (1990). As shown in FIG. 25, injection of haloperidol (i.p. 1 mg/kg) caused the expected induction of c-fos-GFP in the striatum and lateral septum, whereas control animals injected with saline showed minimal c-fos-GFP labeling in these regions (Barth et al., 2004; Wan et al., Brain research 688, 95-104 (1995)).

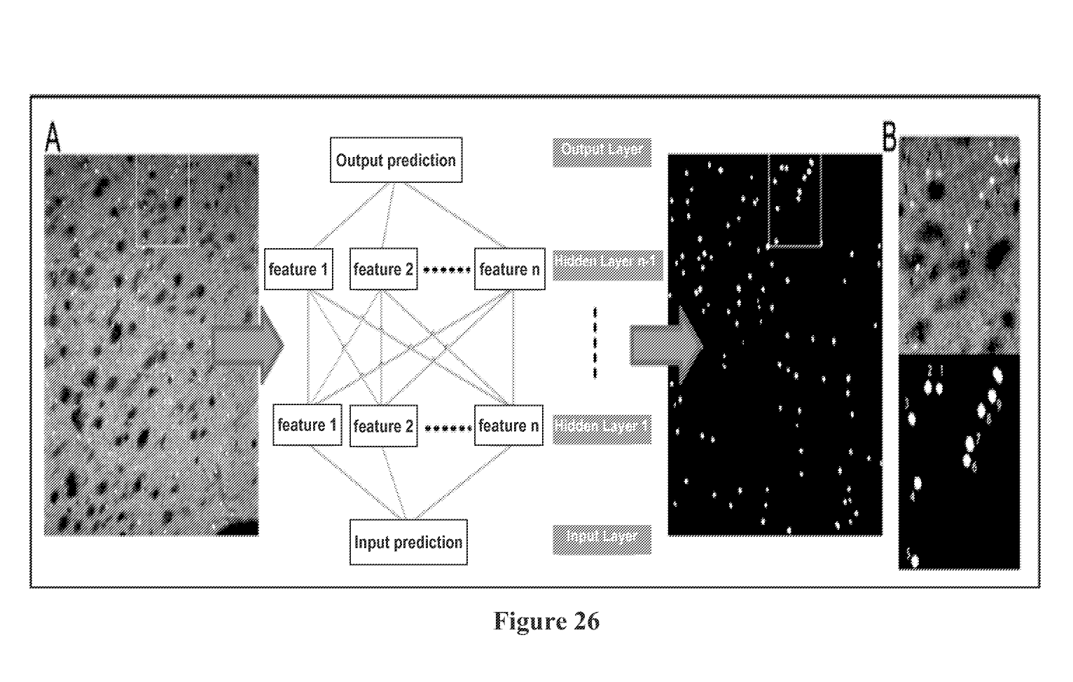

Next, the experimental datasets were used for the training of computational detection by a supervised machine learning approach, namely convolutional neural networks (Jain et al., In CVPR (2010); Turaga et al., Neural computation 22, 511-538 (2010)). Two human observers manually labeled randomly selected tiles to generate ground truth data of c-fos-GFP signal, which were then used to train the convolutional neural networks (FIG. 26). Five fold validation of the training was done. The network had an accuracy of ˜86% of human performance.

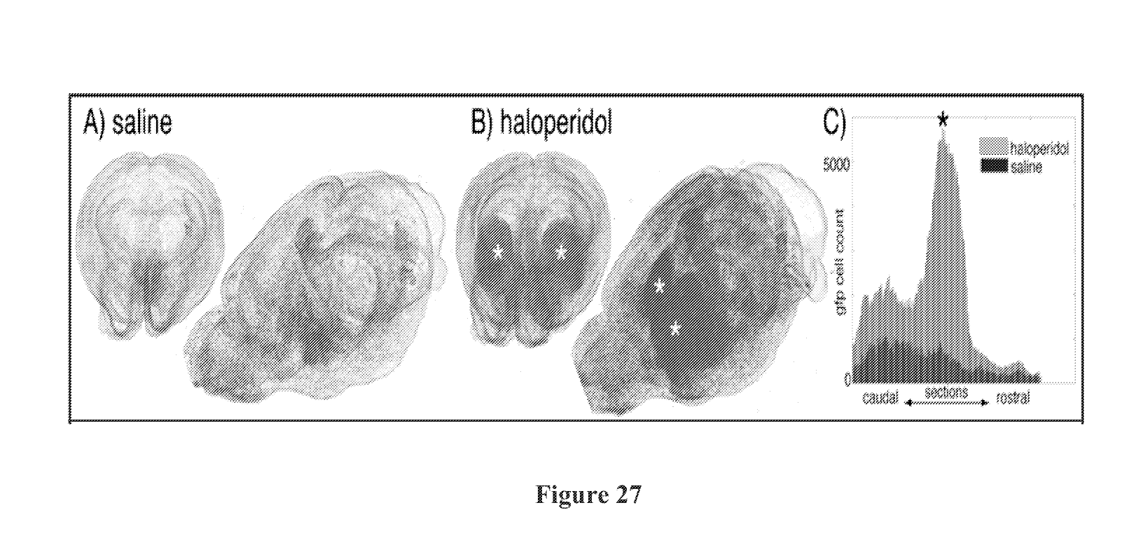

To validate the entire pipeline of data processing, the trained neural networks were applied to extract c-fos-GFP signal in brains of two mice, one injected with saline and the other with haloperidol at 1 mg/kg (FIG. 27). Three hours later, the mice were euthanized, their brains imaged by whole-mount two-photon microscopy, and c-fos-GFP-positive cells were computationally detected and visualized as a three-dimensional representation of intensity centroids (FIG. 17). This experiment revealed the expected strong induction of c-fos-GFP in the caudate putamen (striatum; marked by asterix in FIGS. 27B and C) (Dragunow et al., 1990), as well as increased numbers of c-fos-GFP-positive cells in many caudal coronal sections (FIG. 27C).

These experiments were performed with one animal per treatment, and they demonstrate the 3D representation of the extracted data.

Statistics: Comparison of c-Fos-GFP in Mouse Brain Datasets.

The following approach was established for statistical comparisons between samples. Computationally extracted datasets (FIG. 27) are morphed to a high-resolution MRI atlas (FIG. 24), in order to register the distribution of the c-fos-GFP signal within a standardized brain volume. Second, the brain volume is voxelized to generate discrete digitization of the continuous brain space. Next, an initial comparison is carried out with a set of t-tests applied to each voxel in order to identify “hotspots” of possible differences between separate treatment groups (note that the voxel size is chosen arbitrarily, and datasets segmented at 50, 100 and 200 cubic micrometers are to be compared). Obtaining significant p-values in this manner, however, is not possible due to the large number of multiple comparisons. Instead, statistical analyses developed for functional brain imaging datasets are used, such as order statistics based on random field theory (RFT). The RFT approach takes advantage of the inherent correlation structure between neighboring voxels to reduce the thresholds required for determining significance in the tests between groups (Nichols & Hayasaka, Statistical methods in medical research 12, 419-446 (2003)). Finally, the identified regions of statistical differences are anatomically annotated, using both the segmentation of the MRI atlas and visual analysis of the corresponding raw image data.

This data demonstrate the validity of the method pipeline for high-throughput analysis of IEG induction in the mouse brain. The pipeline has been tested at a throughput of 2 mouse brains per day.

### 6.4 Example 4

### Generation of c-Fos-Based Whole-Brain Representations of Neural Activation Evoked by Antipsychotic Drugs in Wild Type Mice

This example transforms traditional methods of mapping c-fos expression in the mouse brain into an unbiased, high-throughput and high-resolution drug-screening assay.

### Experimental Design

In this example, six antipsychotic drugs that were previously tested by c-fos induction in the rodent brain (Table 2) have been selected for:
1) identifying divergent potencies of individual drugs in the previously identified brain regions (potency is the number of GFP-positive neurons per brain area per drug dose); and
2) discovering new regions of brain activation that failed to be detected in the past. The following experimental procedures are used:
1) Male mice (8 weeks old) are single-housed for one week, during which the mice are briefly handled (restrained in hand and returned to the home cage) once a day. This treatment is designed to limit the baseline expression and variability of c-fos-GFP induction by handling. The number of animals used and the type of transgenic animals used in these experiments can vary.
2) All drugs are injected intraperitoneally (i.p); control mice are injected i.p. with saline;
3) After the injection, the mice are returned to their home cage and euthanized after 3 hours (this time interval was determined as optimal for c-fos-GFP fluorescence in response to haloperidol in pilot experiments).
4) The brains are fixed by transcardial perfusion with 4% formaldehyde, extracted and prepared for whole-mount microscopy described in Example 3.

The drugs are tested as follows:
1) each drug is tested at four doses (Table 2) and compared to saline control;

TABLE 2

| | Dose (mg/kg) | | | | | |
|---|---|---|---|---|---|---|
| | H | F | C | R | O | Q |
| saline, | 0 | 0 | 0 | 0 | 0 | 0 |
| Low | 0.1 | 0.1 | 1 | 0.1 | 0.1 | 1 |
| Medium 1 | 0.4 | 0.2 | 2 | 0.4 | 0.4 | 2 |
| Medium 2 | 1 | 0.5 | 5 | 1 | 1 | 5 |
| high | 5 | 2 | 20 | 5 | 15 | 10 |

Dose curves of haloperidol (H), fluphenazine (F), clozapine (C), risperidone (R), olanzapine (O), and quetiapine (Q). Bradford et al., Psychopharmacology 212: 155-170 (2010), Dawe et al., Neuroscience 171: 161-172 (2010), Moore et al., The Journal of Pharmacology and Experimental Therapeutics 262: 545-551 (1992); Ozaki et al., Eur. Neuropsychopharmacol. 7: 181-187 (1997); Philibin et al., Psychopharmacology 203: 303-315 (2009).

2) each dose is administered to 6 mice, resulting in 5×6=30 brains per drug. The total number of brains for all six antipsychotic drugs is 6×30=180. Each instrument to be used has a throughput of one brain per day at sampling rate of 280 coronal sections (as shown in FIG. 27). The number of test animals per dose can be increased to reach statistical significance for some drugs or add more dose response curve data points, depending on the results.

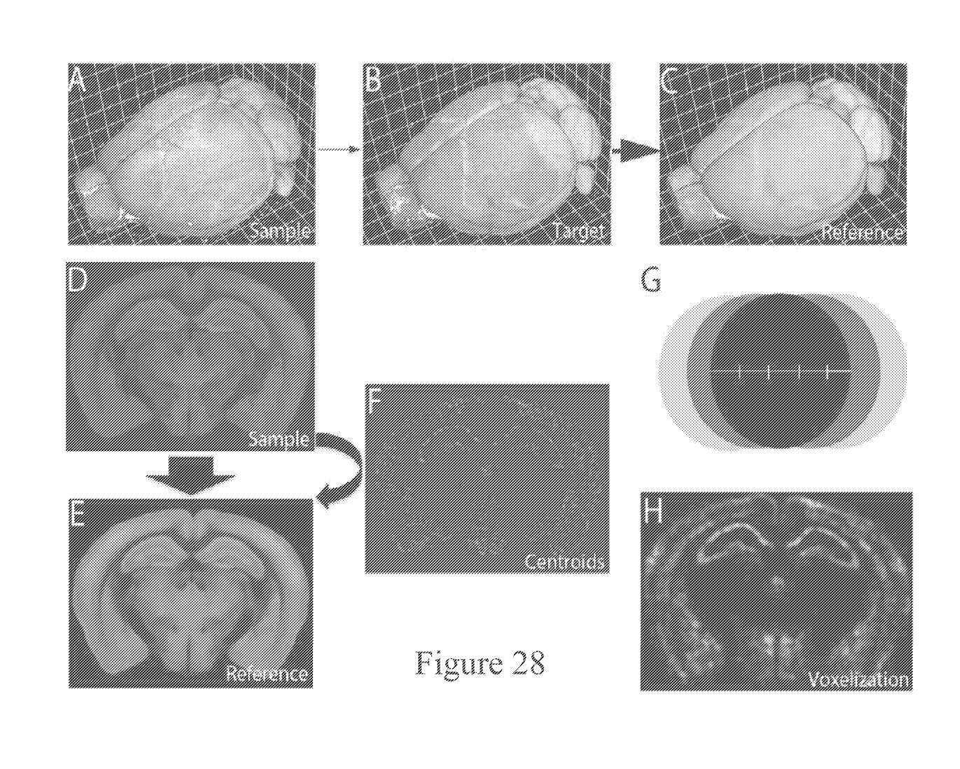

The brains are imaged and computationally processed as described in Example 3. Brains morphed to the MRI atlas (Dorr et al., NeuroImage 42, 60-69 (2008)) are first compared at the level of voxelized brain volumes (see FIG. 28), in order to identify areas of significant c-fos-GFP induction in drug versus control samples. Once such areas are determined, anatomical regions comprising the voxels with activated cells are marked up. In some cases, it is possible to directly infer the anatomical areas from the MRI atlas, which comprises segmentation of 62 brain regions (Dorr et al., 2008). However, small brain structures need to be manually outlined within the MRI template based on morphing of the obtained scans with the MRI atlas and the Allen Mouse Brain Reference atlas (Lein et al., Nature 445, 168-176. (2007)).

The data from these experiments is organized as a spreadsheet containing the numbers of activated GFP-positive neurons (after subtraction of GFP counts from control brains) in anatomical brain regions for each drug in a dose response curve.

### 6.5 Example 5

### Analysis of Antipsychotic Drugs in the Mouse Brain by High-Throughput Microscopy of c-Fos Expression

In this example, c-fos mapping was used in a quantitative, high-resolution, automatic method to screen drugs.

This example analyzes the effects of antipsychotic drugs on neural circuit activity in the whole mouse brain. The method comprises the following steps: (1) an automated whole-brain microscopy, STP tomography, was used to image brains of c-fos-GFP mice, which express GFP as a marker for native c-fos; (2) the distribution of the activated c-fos-GFP-positive neurons was computationally detected by convolutional neural networks; (3) the processed datasets were warped and registered in a 3D reference brain and voxelized for statistical comparisons. In particular, this example demonstrates the application of the described method for screening haloperidol, a typical antipsychotic.

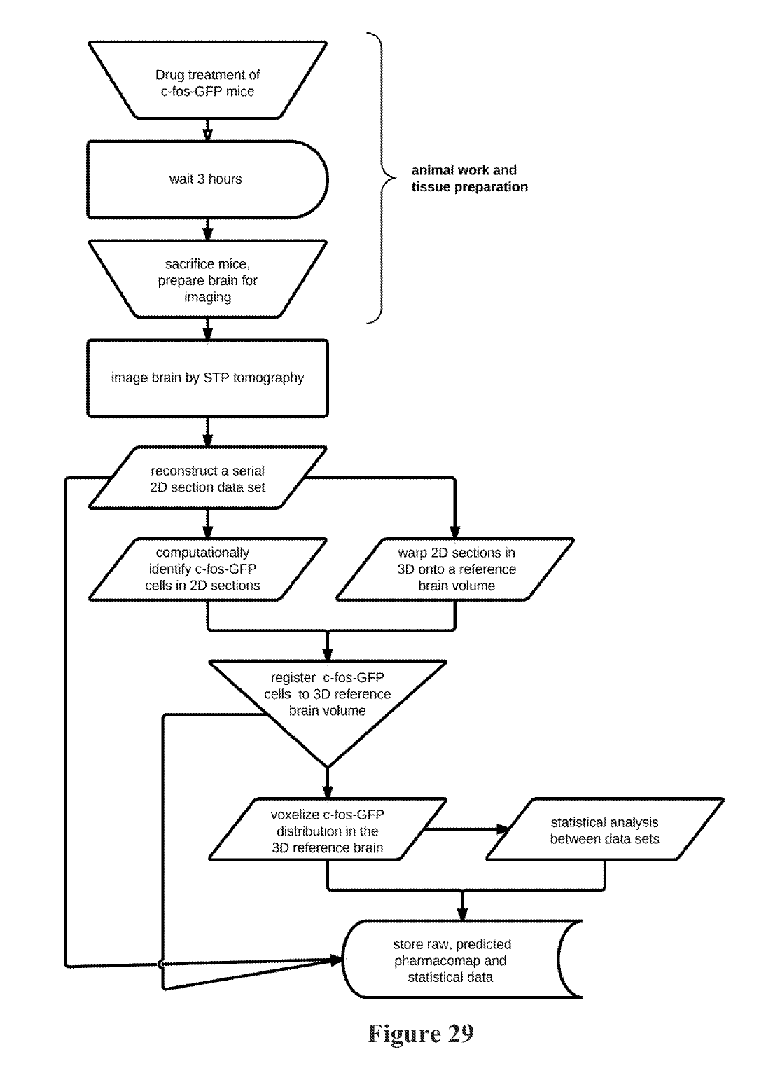

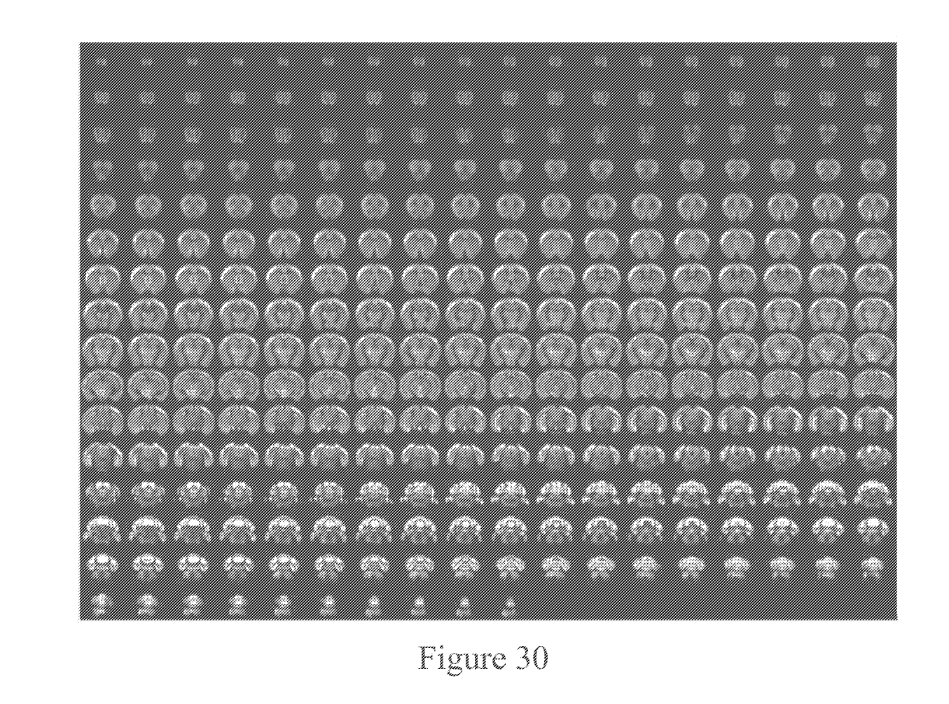

FIG. 29 shows a schematic flowchart of the experimental design. The experiment was performed as follows:

Animal Work and Tissue Preparation.

Transgenic c-fos-GFP mice (Reijmers et al., Science 317:1230-1233 (2007)), expressing GFP as a surrogate marker for native c-fos, was injected with intraperitoneally with haloperidol (1 mg/kg) or saline (control). The mice were returned to their home cage and left undisturbed for 3 hours, a time period needed for the induction and fluorophore maturation of c-fos-GFP. Next, the mice were deeply anesthetized and euthanized by intra-cardiac perfusion with saline and paraformaldehyde for brain fixation. The mice were decapitated and the brain was extracted, postfixed and embedded in agar for STP tomography. The instrument used for STP tomography was essentially the same as that shown in FIG. 22. Three PMTs (C1-C3) can be used for multi-color imaging.

Reconstruction of a Series 2D Sections.

The imaged brain was reconstructed as a series of 2D sections, typically 280 to 300 per one mouse brain as shown in FIG. 30.

Computational Detection of c-Fos-GFP.

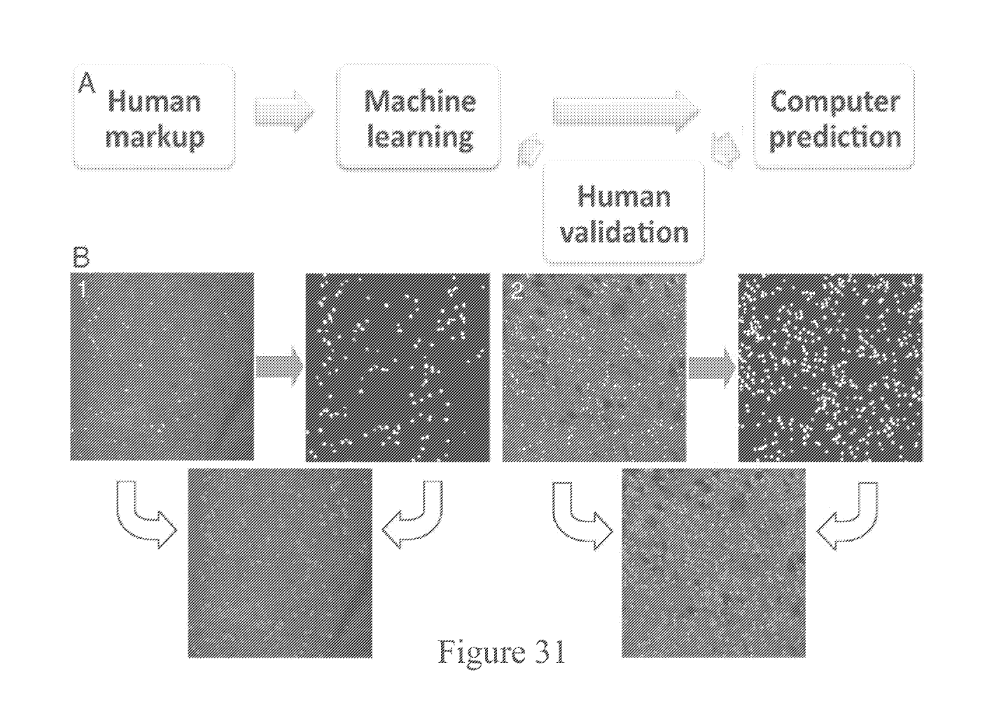

Convolutional neural networks (Turaga et al., Neural computation 22:511-538 (2010)) learned inclusion and exclusion criteria of c-fos-GFP labeling based on human markups as shown in FIG. 31A. Then, c-fos-GFP was detected (examples of s-fos-GFP detection are shown in FIG. 31B.

Raw Data Warping to a Reference Brain Atlas.

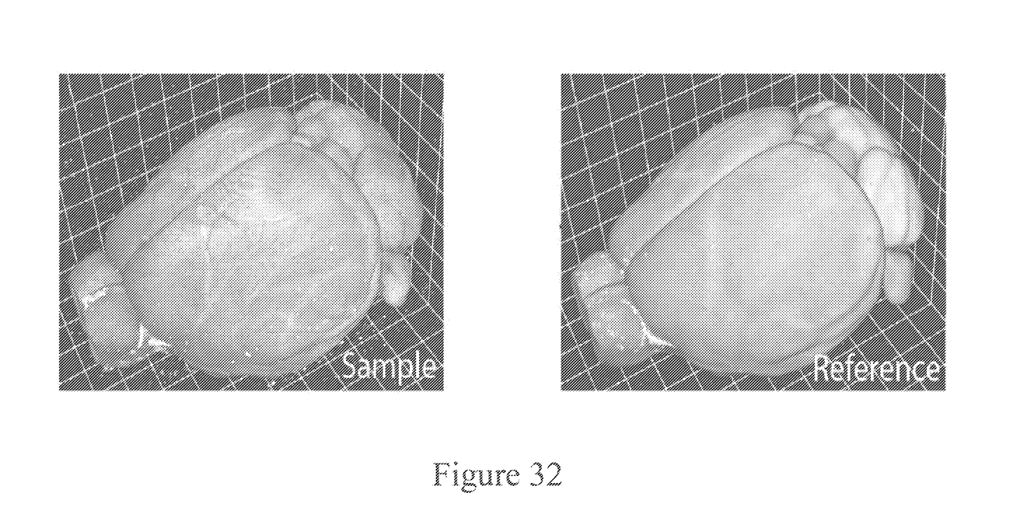

The serial 2D-section data set was reconstructed in 3D and warped onto a 3D reference brain volume generated as an average of twenty wild type brains scanned by STP tomography as shown in FIG. 32. The warping was done based on tissue autofluorescence, using elastix software.

c-Fos-GFP Data Registration to a 3D Reference Brain.

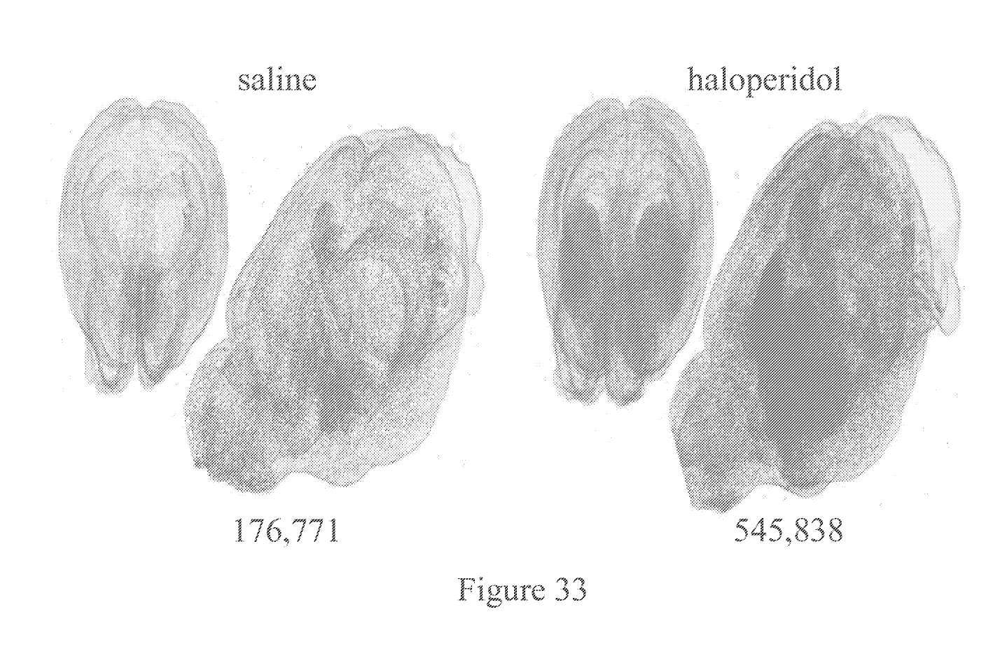

Registration of c-fos-GFP data onto the reference brain created a 3D representation of c-fos-GFP distribution, a c-fos-GFP pharmacomap. c-fos-GFP pharmacomaps of saline and haloperidol (1 mg/kg) brains with 176,771 and 545,838 c-fos-GFP cells, respectively, are shown in FIG. 33.

Voxelization of 3D c-Fos-GFP Data.

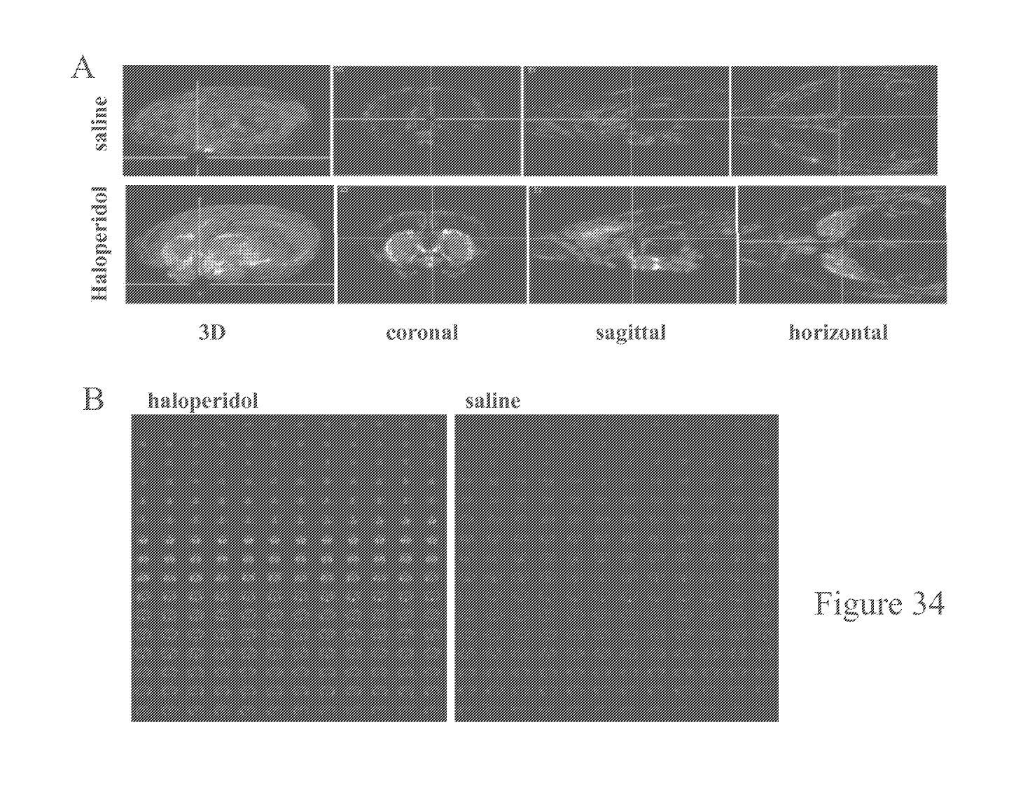

The 3D brain volumes were voxelized as an evenly spaced grid of X-Y-Z=450×650×300 voxels, each voxel of size 20×20×50 microns, to generate discrete digitization of the continuous brain space. In FIG. 34, top two rows show heat-map distribution of c-fos-GFP in voxelized saline and haloperidol brains in 3D (FIG. 24A), and the bottom panels show the same brains in 2D montage (FIG. 34B).

Statistical Comparison.

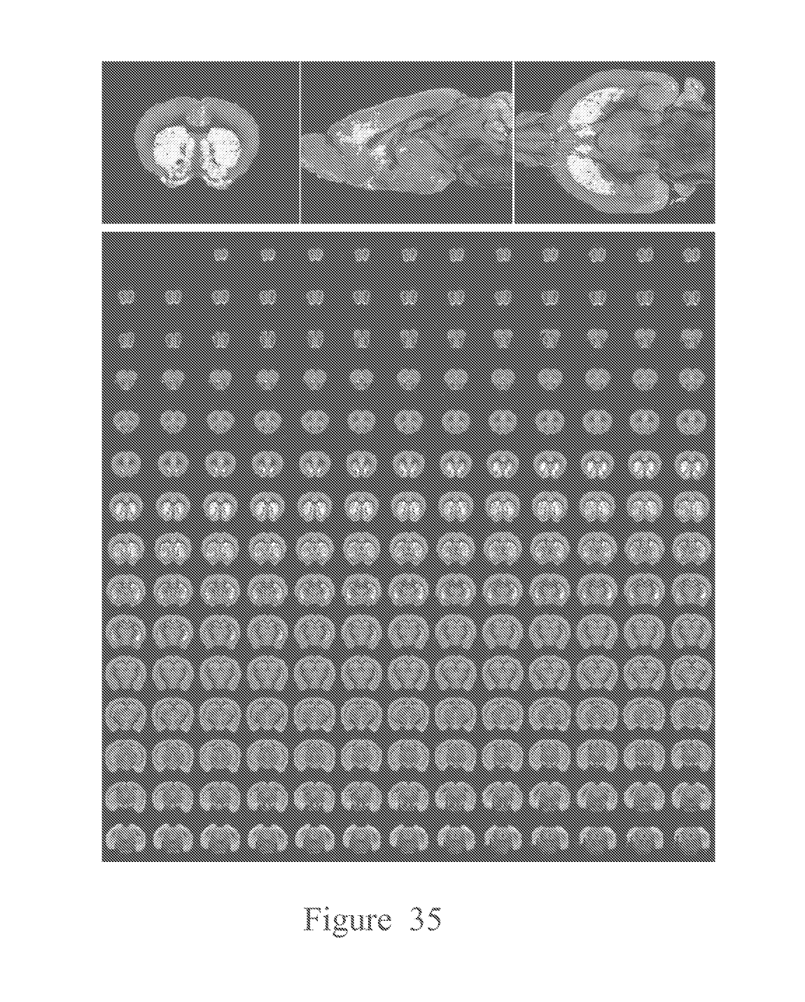

FIG. 35 shows heat maps of statistical differences between haloperidol (n=7) and saline (n=7) injected mice. Statistical comparison between the two groups was done by a series of negative binomial regressions. Type I error was corrected by setting a false discovery rate (FDR) of 0.01, under the assumption that the voxels have some level of positive correlation with each other.

Results.

This example demonstrates that all brain regions identified previously were detected using the described methodology: Medial prefrontal Cx, Cingulate Cx, Piriform Cx, Major Islands of Calleja, Nc Accumbens (whole, shell, core), Lateral septum, Striatum (whole), Medial preoptic area, Paraventricular nucleus, Bed nucleus of stria terminalis, Medial thalamus (Sumner et al., Psychopharmacology 171, 306-321 (2004)). Further, additional areas, that have not been previously identified, were detected using the described methodology. Additional areas of statistical differences included: Dorsal tenia tecta, Dorsal peduncular Cx, Ventral pallidum, Olftactory tubercle, Indusium griseum, Motor Cortex, Reunions thalamic nc, Centrolateral thalamic nc, Dorsomedial hypothalamic nc, Medial parietal association Cx, Parietal Cx, Primary and Secondary auditory Cx, Arcuate hypothalamic nc, Ectorhinal Cx, Posterior hypothalamic nc, Substantia nigra compacta, Subiculum, Amygdalopiriform transition, Med mammillary nc, Retrospenial granular Cx.

This example shows that the described methodology provides the first automated and unbiased method for mapping drug-evoked activation in the whole mouse brain at cellular resolution. Specifically, the current experiments demonstrate a quantitative and standardized analysis of haloperidol-induced brain activation, reproducing previous results and identifying a number of new areas of action. Thus, screening of drugs used in the clinics (with known human outcome) using the described method will allow generation of a “template” or reference database of c-fos-GFP pharmacomaps, which may be used for quantitative comparisons of new drugs in preclinical Research and Development.

### 6.6 Example 6

### C-Fos-Based Whole-Brain Analysis of Social Behavior-Evoked Neural Activation in Mouse Models of Autism

Impaired social interaction is the hallmark feature of autism spectrum disorders. In this example, genetic mouse models of autism were used to identify brain circuitry involved in social behavior and to examine how these circuits are affected by autism candidate gene mutations. c-Fos, an immediate early gene that is induced in response to various forms of external stimuli, was used as a reporter for brain activation during social interaction. The analysis of c-fos induction was done in whole brains by serial two-photon (STP) tomography with c-fos-GFP mice. STP tomography images the mouse brain as a series of coronal sections by combining two-photon mosaic imaging and mechanical sectioning by a built-in vibratome. This method thus allows examining c-fos-GFP change throughout the entire mouse brain, which helps to systematically examine brain areas with increased c-fos-GFP labeling after social behavioral stimulation. Brain circuits in autism mouse models were analyzed. Results show that neuroligin 3 R451C mutant mice and neuroligin 4 knockout mice, compared to respective wild type littermates, failed to show increased c-fos in several brain areas after social exposure.

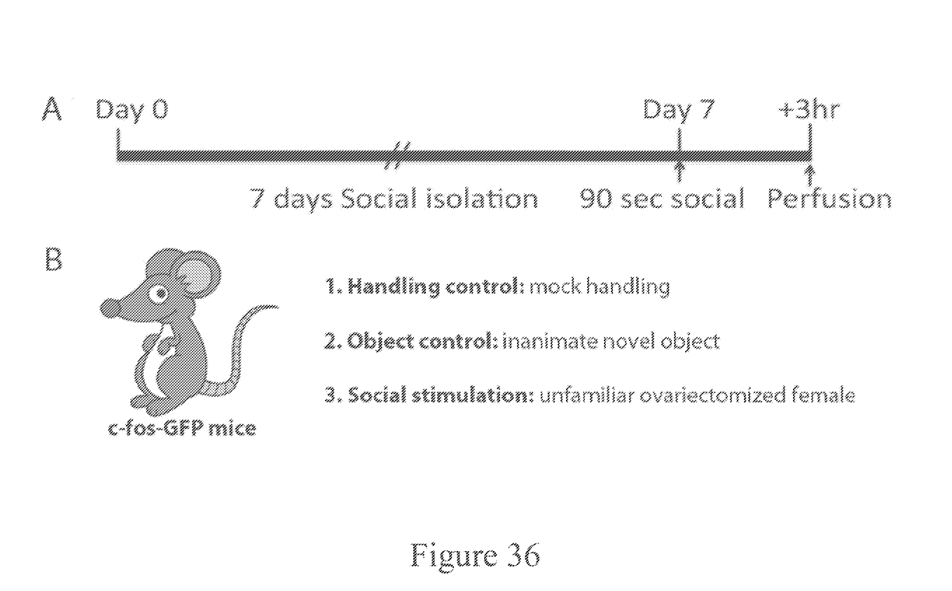

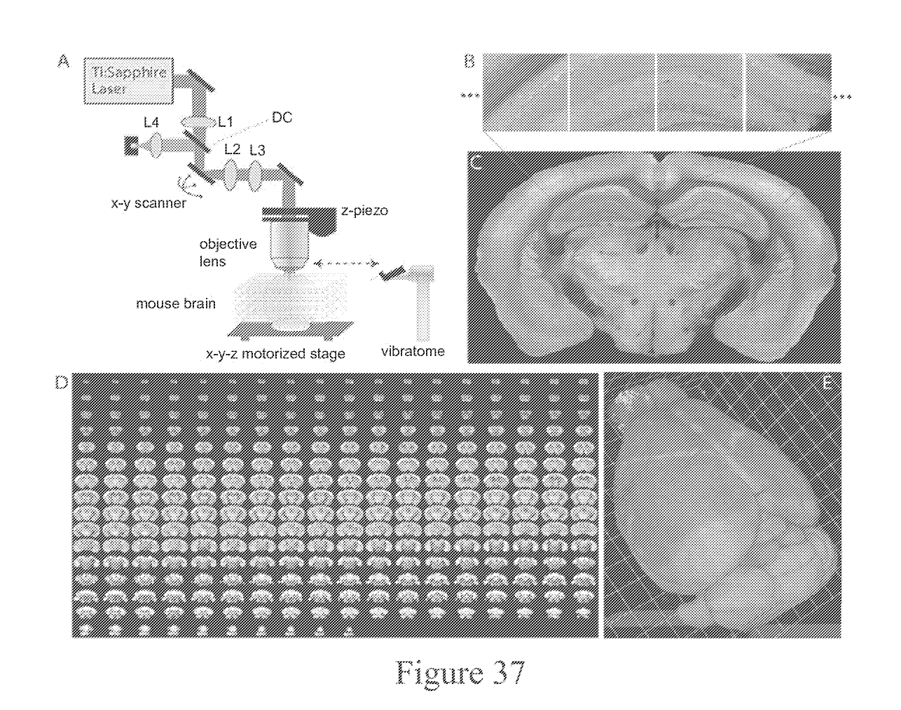

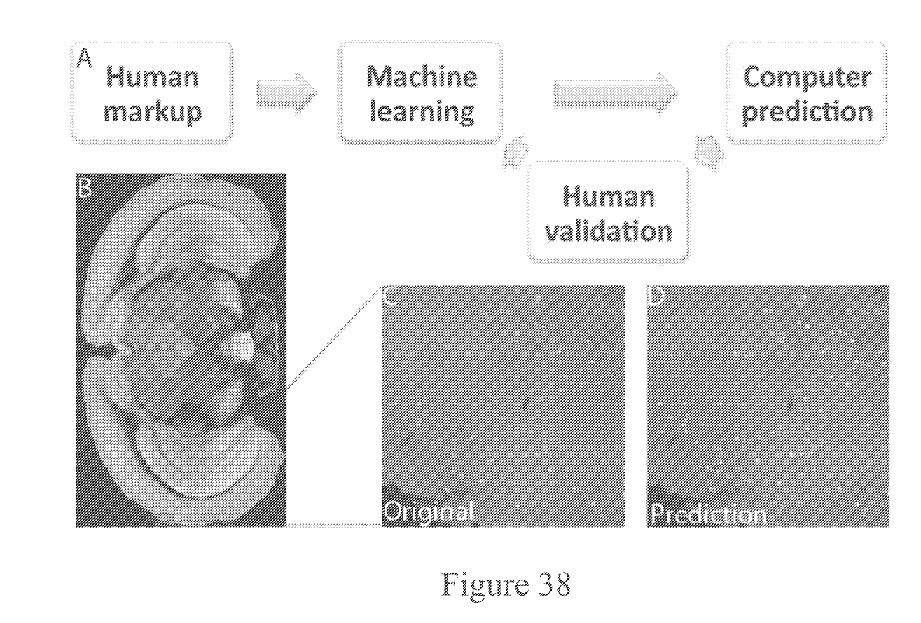

To investigate social brain circuitry, mice kept in social isolation for 7 days were subjected to 90 seconds of a social stimulation. Three different groups of mice were used: handling control (mock handling), object control (inanimate novel object), and social stimulation (unfamiliar ovariectomized female); 7 mice per group were used. After 3 hours post-stimulation mice were sacrificed and perfused. Experimental design is shown in FIG. 36. Next, serial two-photon tomography was used to examine entire brain with cellular resolution (see FIG. 37 showing 3D reconstruction of an entire brain using STP tomography). Then, machine learning algorithm was used for automatic detection of c-fos-GFP cells (see FIG. 38, showing that first computer learns inclusion and exclusion criteria of c-fos-GFP cells based on initial human markup, and then detects the positive cells automatically for new data set (prediction)).

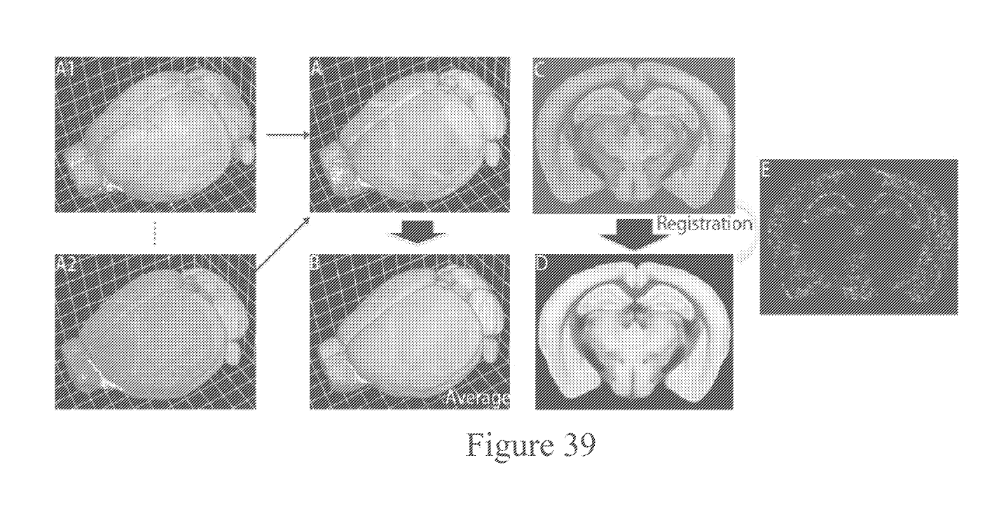

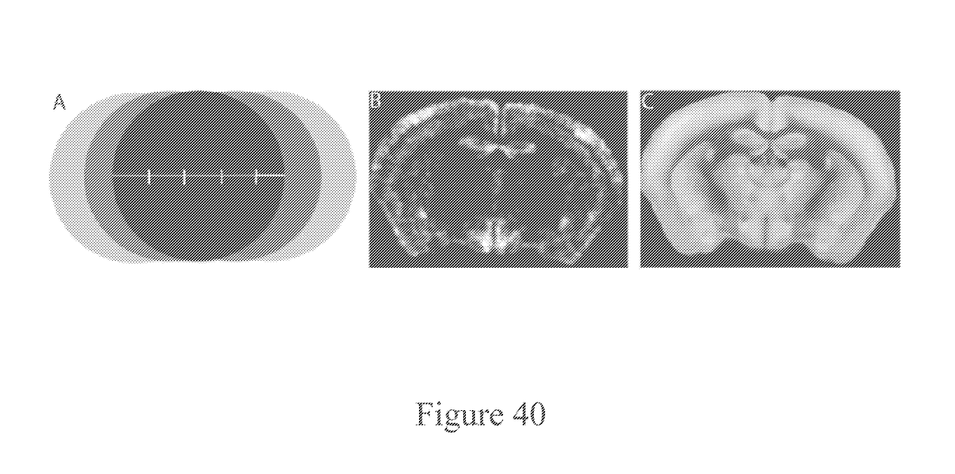

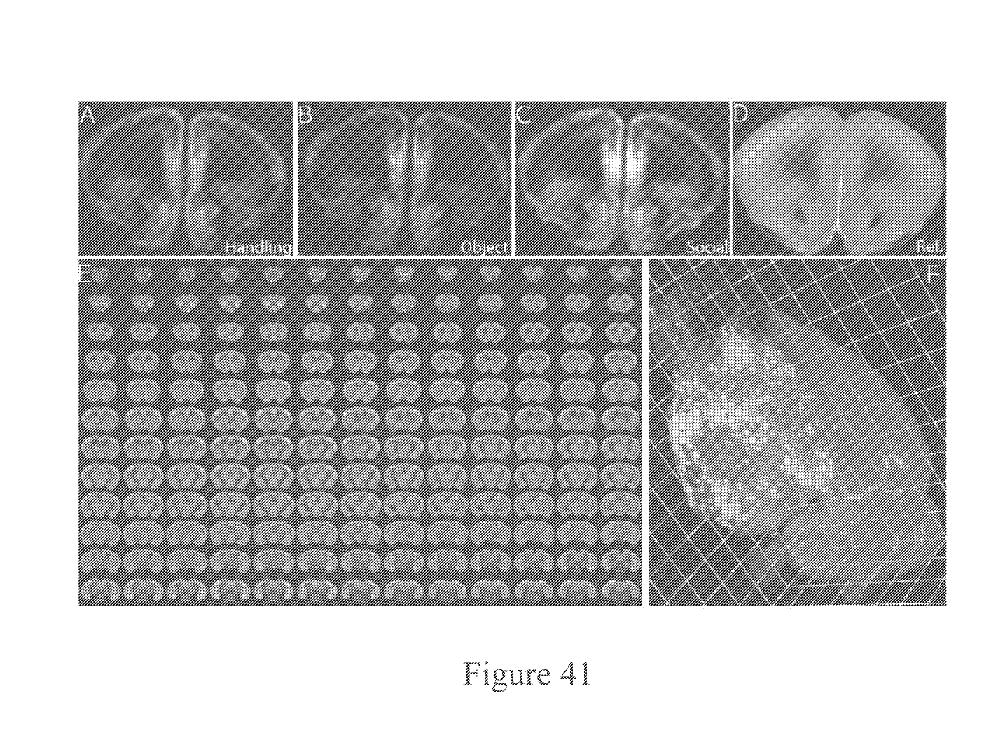

Subsequently, image registration to a reference brain was performed (see FIG. 39 showing that 19 different brains (A1 and A2) were registered to one brain (A) to generate a reference brain (B) (average of 20 brains); and that prediction results (E, centroids of c-fos-GFP cells) were registered to a reference brain (D) based on registration parameter from a sample (C) to a reference brain (D)). Then, voxelization to measure c-fos-GFP cell increase was performed, as shown in FIG. 40, and the voxelized brain image (B) was registered in the same space of the reference brain (C). Next, voxel-wise statistical analysis was performed to identify brain areas responding to social exposure. FIG. 41 demonstrates averaged voxelization results registered to the reference brain (D) from handling control (A), object control (B), and social stimulation (C) group, and a 3D overlay of the activated brain area and the reference brain (F).

The following brain areas were activated by social exposure:

(i) mPFC regions: medial orbital cortex, prelimbic cortx, infralimbic cortex, Cingulate cortex;
(ii) Agranular insular cortex;
(iii) clastrum;
(iv) piriform cortex;
(v) Olfactory tubercle;
(vi) Lateral septum;
(vii) Nucleus accumbens;
(viii) Medial preoptic area;
(ix) Somatosensory cortex;
(x) Amygdala: Basal lateral amygdala, Basal medial amygdala, Medial amygdala, posterior medial cortical amygdale;
(xi) Hypothalamus: Paraventricular hypothalamus, Ventral medial hypothalamic nucleus, Dorsal medial hypothalamic nucleus;
(xii) Dorsal endopiriform nucleus;
(xiii) Premamillary nucleus;
(xiv) Amygdalohippocampal area;
(xv) Visual cortex;
(xvi) Subiculum.

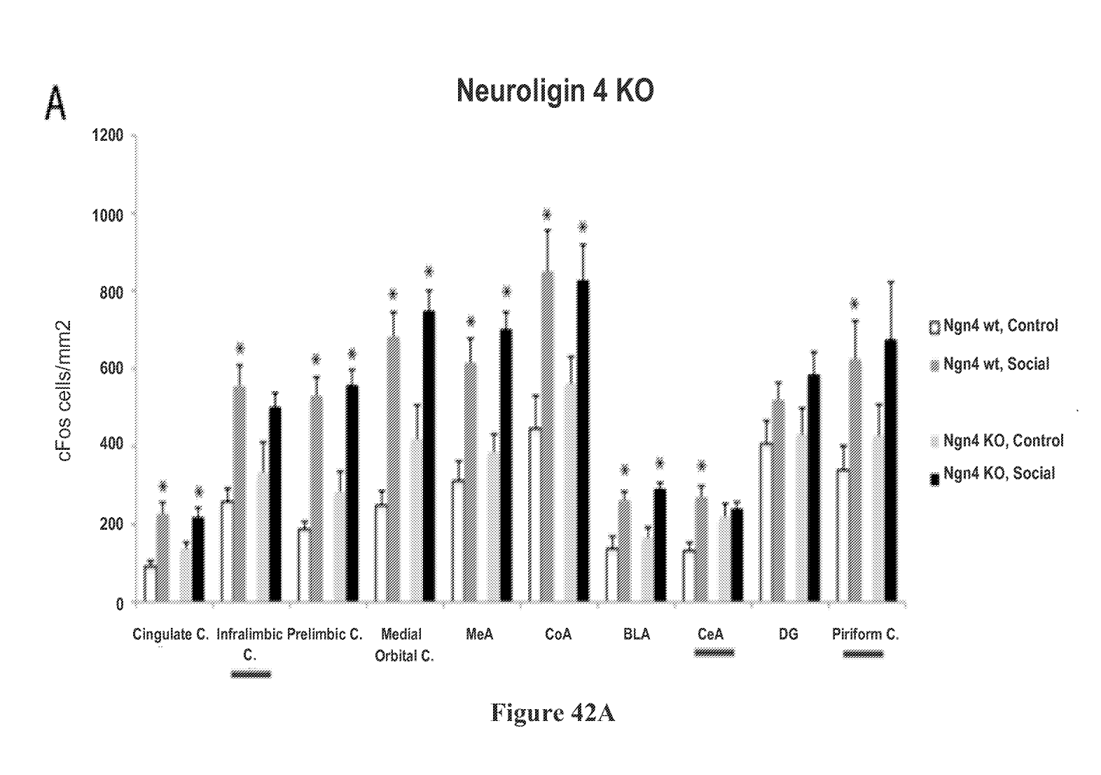

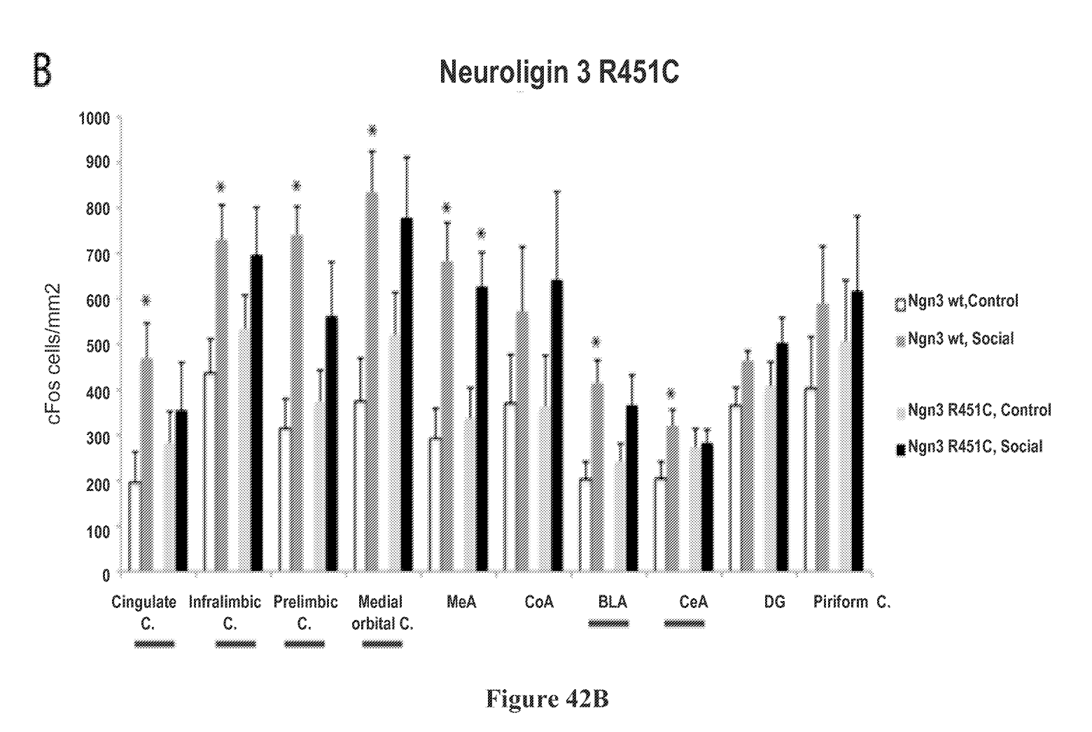

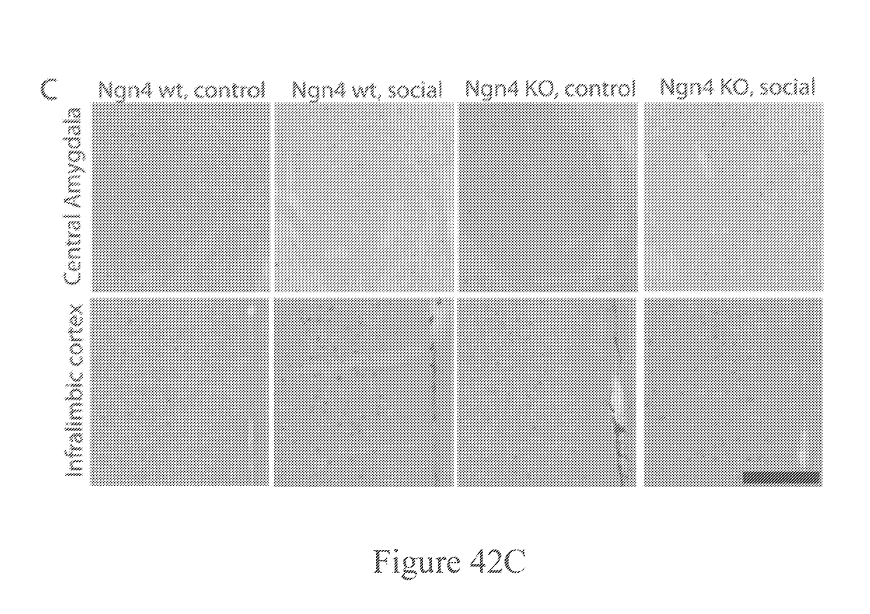

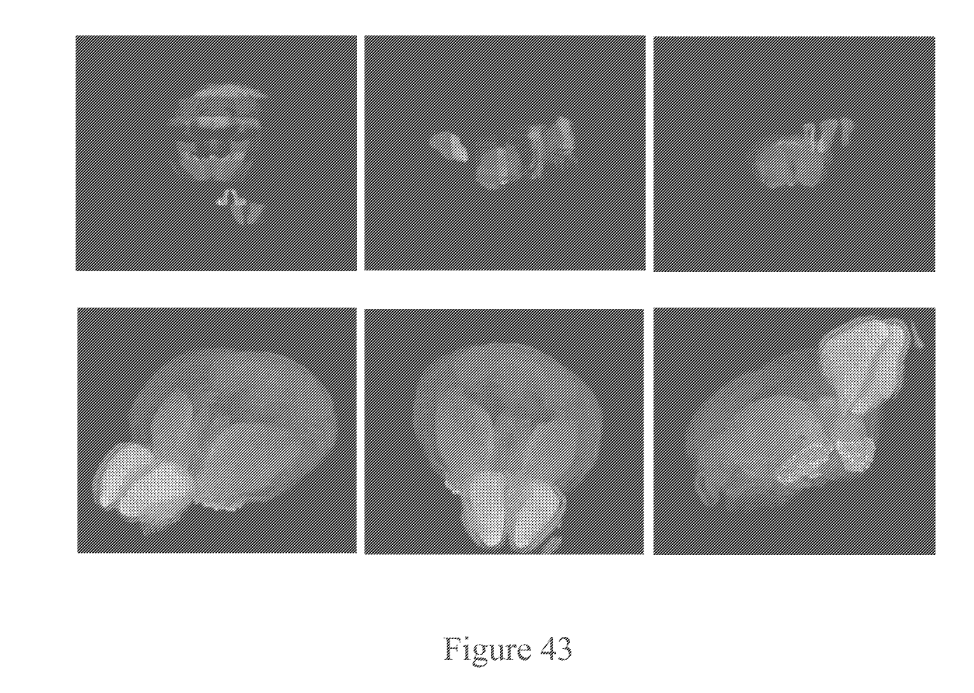

FIG. 42 presents a summary of c-fos density in wild-type mice and in autism mouse models carrying neuroligin 4 KO (A) and neuroligin 3 R451C. It indicates brain areas which have significant c-fos increase in wild type littermates but not in Ngn 4 KO and Ngn 3 R451C. In particular, wild type littermates showed significant increase in central amygdala and infralimbic cortex, whereas neuroligin 4 KO didn't show similar increase after social exposure. FIG. 42 demonstrates that shared brain areas in autism mouse models failed to show significant c-fos increase after social stimulation.

This example shows that a system was created to examine c-fos-GFP changes responding to external stimuli throughout entire brain in an unbiased way. In particular, STP tomography enabled to see c-fos-GFP changes throughout entire brains, and machine learning algorithm could robustly detect c-fos-GFP positive cells automatically. Further, image registration process enabled to compare same brain areas from different brains, and voxel-wise statistical analysis revealed brain areas activated by social exposure. In addition, preliminary c-fos immunohistochemistry studies indicated that specific brain areas fail to get activated by social exposure, suggesting potential converging brain circuits commonly affected by autism candidate gene mutations.

### 6.7 Example 7

### Machine Learning-Based Cell Counting in the Mouse Brain Using Serial Two-Photon Tomography

Until now, the exact numbers of neurons in the whole nervous system have been determined only for simple organisms, such as the C. elegans nervous system. Numbers of neurons in more complex nervous systems, such as the rodent brain, are estimated only approximately, based on interpretations of cell densities from manually counted small brain regions.

In this example, a new method is presented that generates complete numbers of different classes of interneurons in the mouse brain. Double transgenic mice were used with fluorescently labeled nuclei of specific interneuron cell types: mice carrying cell type-specific expression of a Cre recombinase was crossed with fluorescent reporter mice expressing nuclearly targeted EGFP after Cre-based recombination and deletion of a lox-stop-lox cassette. The brains of these mice were imaged by Serial Two-Photon (STP) tomography, which generated complete brain scans at high resolution, such as 1 micron×1 micron×2 micron. Once the entire 3D volume was reconstructed, a trained convolutional neural network was used to predict the nuclear labels. A standard MRI mouse brain was then warped onto the STP tomography datasets along with its labels for anatomical segmentation. Analysis of a complete interneuronal count, using GAD-Cre transgenic mice, is being performed.

3D Image Reconstruction is shown in FIG. 43. The entire brain was imaged in 8 blocks. Each block was scanned just as to encompass the brain region without the fixation medium. The blocks of different slices were aligned to a reference block using Scale-invariant feature transform (SIFT) based method and entire brain was reconstructed in 3D.

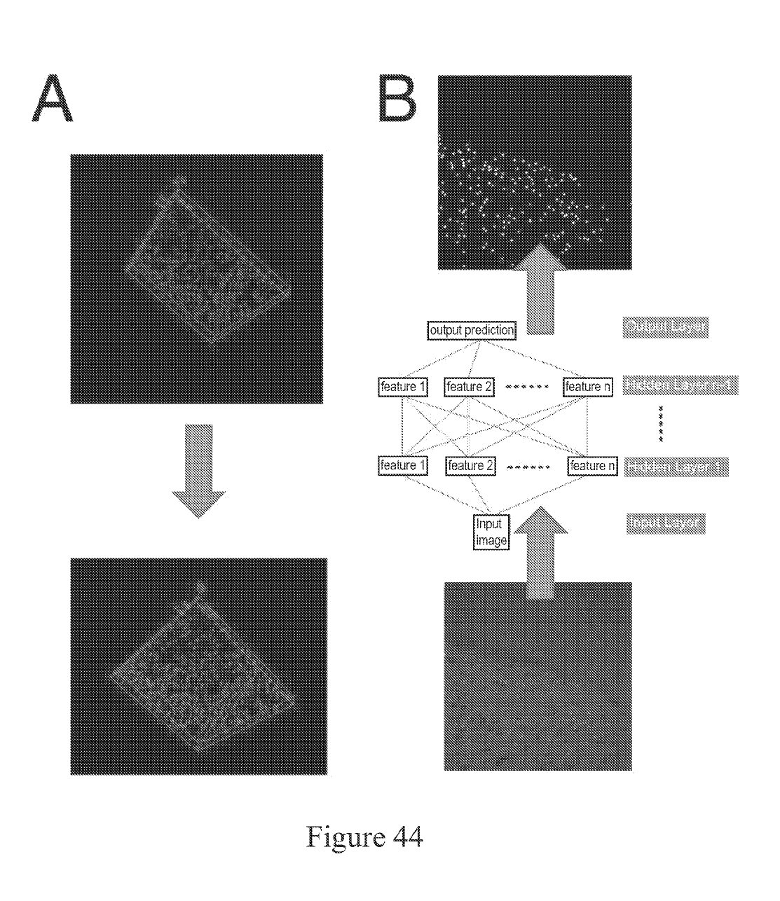

GAD-Cre detection and quantification is shown in FIG. 44. Randomly selected 3D tiles from different regions of the brain were labeled by a human observer for the GAD-Cre signal. This ground truth data was used to train a convolutional neural network for GAD-Cre signal detection. The training was done using a subset of images and then used on the rest of the brain image.

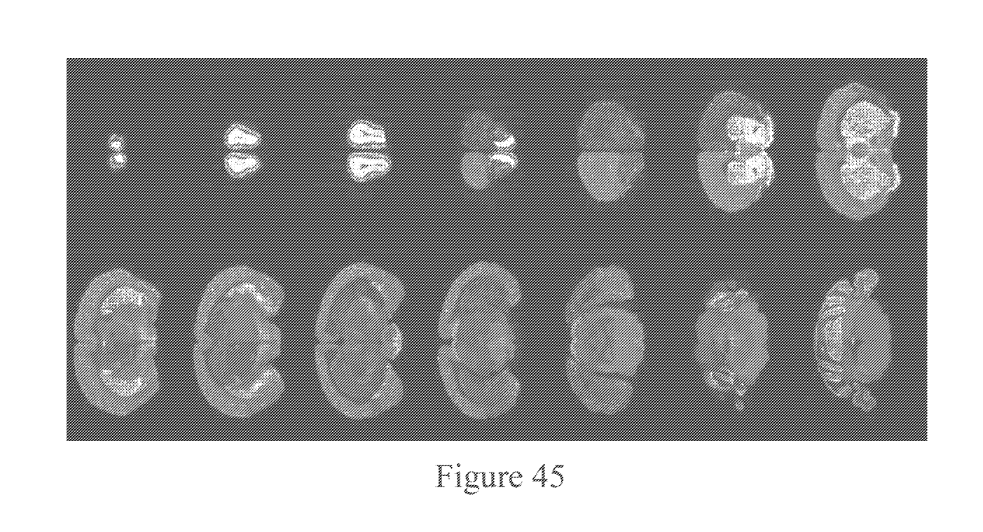

Anatomical Segmentation is shown in FIG. 45. An MRI atlas was warped on to the brain image on the auto-fluorescence channel (resampled at 20 microns in x & y, 50 microns in z) using mutual information as constraint and thus using the same warping parameters; brain region labels were also warped. The resultant label was then resampled to original x, y, z resolutions and region wise counting was done.

Then, reconstruction of the brain surface and plotting of the centroids of the detected GAD-Cre-GFP signals is performed. The brain is imaged in 300 sections, 50 microns apart at a 1 micron lateral resolution.

The described method enables studying of complex brains using STP tomography imaging combined with computational detection of fluorescently labeled nucleus of GAD-Cre knock-in mice.

### 6.8 Example 8

### Generation of a Pharmacomap

FIG. 46 illustrates an example process for generating a pharmacomap of a drug. In this representative example, c-fos expression is mapped. The example process includes steps A-H for generating the pharmacomap. At step A, c-fos-GFP transgenic mice (Yassin et al., Neuron 68:1043-1050 (2010)) are injected (e.g., intraperitoneally) with the drug. Control mice are injected (e.g., intraperitoneally) with saline. For example, before injection, male mice (8 weeks old) are single-housed for five days in order to limit the variability of the baseline c-fos-GFP expression. At step B, the mice are euthanized after a predetermined time period (e.g., 3 hours) to allow peak c-fos-driven GFP expression. At step C, the mouse brains are fixed (e.g., by transcardial perfusion with 4% formaldehyde), extracted and prepared for STP tomography, and drug-evoked activation in the mouse brains is imaged at cellular resolution (Ragan et al., Nature Methods 9:255-258 (2012)). Then, at step D, whole-brain datasets are generated from the images of the mouse brains. For example, a c-fos-GFP brain is imaged as a dataset of 280 coronal sections by STP tomography which integrates two-photon microscopy and tissue sectioning.

At step E, the c-Fos-GFP-positive neurons are detected by machine learning algorithms (e.g., by neural-network-based algorithms) in order to generate brainwide “heat maps” of statistically significant differences in c-fos-GFP cell counts. For example, c-fos-GFP signal is analyzed by convolutional neural networks that were trained to recognize inclusion and exclusion criteria of the nuclear c-fos-GFP labeling based on initial human markups (Turaga et al., Neural computation 22:511-538 (2010)). After 5-fold validation of the training, the computer-based prediction reached a performance level comparable to human inter-observer variability, with ˜10% type II error (a failure to detect weakly labeled cells with low signal-to-noise ratio) and a very low type I error (detection of false positive cells). The convolutional neural networks thus provide an automated and highly accurate detection of c-Fos-GFP-positive cells in STP tomography datasets.

A 3-dimension (3D) brain-wide c-Fos-GFP distribution is reconstructed at step F. At step G, the datasets are warped (e.g., co-registered) on to a standard “reference” brain volume and voxelized for statistical comparisons. For example, the “reference” mouse brain is generated by averaging the tissue autofluorescence signal of twenty wild type brains by the ITK elastix software (Klein et al., IEEE Transactions on Medical Imaging 29:196-205 (2010)). The same tissue autofluorescence signal of each future dataset is then used to warp the dataset to the reference brain and to register the computer-generated prediction of c-Fos-GFP distribution. Once all data are warped to the reference brain, the 3D brain volume is voxelized to generate discrete digitization of the continuous space. For example, the datasets are represented as the number of centroids (c-fos-GFP cells) lying within an evenly spaced grid of 450×650×300 elements (voxels), each of size 20×20×50 microns.

Further, at step H, c-Fos-GFP distribution in voxelized control and experimental brains is compared to determine the anatomical brain regions with significant differences in c-Fos-GFP expression in order to generate the pharmacomap. For example, a series of negative binomial regressions can be performed to detect the differences between different drug groups. Because the test is applied to every voxel location, even with a low type I error rate, there will be a large number of locations where the test result is significant, but there is no real physiological difference between the experimental groups. A false discovery rate (FDR) is set to 0.01, under the assumption that the voxels have some level of positive correlation with each other. The negative binomial regression analysis reveals “hot-spots” of statistical differences between groups. Such areas are next anatomically identified, using of a reference atlas (e.g., the Allen Reference Atlas (Hawrylycz et al., PLoS computational biology 7, e1001065 (2011))) co-registered with the reference brain.

Some drugs being tested may have more variable effects on brain activation in mice than others. In addition, the intraperitoneal drug delivery itself can result in some variability even in the hands of an experienced experimentalist. Anatomical segmentation of the pharmacomap, however, allows determining the standard deviation (SD) of the drug-induced c-Fos activation across different brain regions. The variability of the drug-evoked response can be monitored and, for example, extra animals can be added to the drug group in case of higher than usual SD, in order to achieve more uniform estimates of the mean. In addition, the mice can be video-monitored for 30 minutes before and the entire period (e.g., 3 hours) after the drug delivery (before the animal is euthanized for STP tomography) and the recording can be automatically analyzed for a set of standard home cage behaviors. Therefore, a highly atypical behavioral response, for example due to mistargeting the injection, would be detected and the particular case would be triaged before analyzing the data.

Furthermore, as an example, pharmacomap patterns may be combined with information about structural, physical, and chemical properties (SPCPs) of drug compounds. The information about the 3D conformation of molecules is available from PubChem, in the form of SDF files, and can be submitted to the EDRAGON online computational chemistry tool (Tetko and Tachuk, Virtual Computational Chemistry Laboratory (2005)) to evaluate the SPCPs. A set of SPCPs can be added for every chemical to the set of neural responses that defines pharmacomaps. SPCPs can be included in addition to pharmacomaps to improve the quality of prediction, and may also reveal drug-structure-related rational drug-design principles.

### 6.9 Example 9

### Generation of Haloperidol, Risperidone, and Aripiprazole Pharmacomaps

This example demonstrates the ability to generate pharmacomaps for three different drugs and to compare the pharmacomaps to obtain information regarding activation evoked by the drugs in the mouse brain at cellular resolution.

Typical and atypical (second generation) antipsychotics represent a good example of the complexity of clinical effects and side-effects shared by drugs of the same therapeutic family. The typical antipsychotic haloperidol (mainly D2 antagonist) is often reserved solely for the treatment of acute, severe psychosis, mainly due to its strong extrapyramidal side-effects (EPSEs) (Irving et al., Cochrane Database of Systematic Reviews 4 (2006)). In contrast, atypical (second generation) antipsychotics cause EPSEs much less frequently and are often prescribed for broader indications. For example, risperidone (mainly D2/5HT2A antagonist) is used to treat manic states in bipolar disorder and irritability in autism (Scott et al., Pediatric Drugs 9, 343-354 (2007)), but can cause weight gain, somnolence, and hyperprolactinemia among others (Komossa et al., Cochrane database of systematic reviews (online), CD006626 (2011); Kuhn et al., Molecular systems biology 6, 343 (2010)). Aripiprazole (mainly D2/5HT2A antagonist and 5HT2A partial agonist) is used to treat bipolar disorder, major depressive disorder and irritability in autism (Farmer et al., Expert opinion on pharmacotherapy 12, 635-640 (2011)), but can cause headache, insomnia, nausea, and fatigue among others (Kuhn et al., Molecular systems biology 6, 343 (2010)).

In this example, as shown in FIG. 47, pharmacomaps (e.g., A, B, and C) for haloperidol, risperidone, and aripiprazole, respectively, were generated to assay the mouse-brain activation evoked by the three antipsychotics at a moderate dosage: haloperidol 0.25 mg/kg, risperidone 1.0 mg/kg, and aripiprazole 1.0 mg/kg. 5 mouse brains were used for each drug. As shown in Table 3 and FIG. 47, statistical comparisons between the three drugs' pharmacomaps and that of control (saline-injected) mice identified the common activation of caudate, putamen, and nucleus accumbens that has been previously well described in both mice and humans (Natesan et al., Neuropsychopharmacology 31:1854-1863 (2006), and Mawlawi et al., J. Cerebr Blood Flow Metab 21:1034-1057 (2001)). In addition, the three pharmacomaps revealed a remarkable level of differential cortical and subcortical activation patterns unique to each drug.

As shown in pharmacomap A for haloperidol, haloperidol activated a major portion of the caudate putamen (CP) and nucleus accumbens (ACB), as well as the olfactory tubercle (OT), prelimbic cortex (PL), lateral septum (LS) and dorsomedial hypothalamus (HYP). As shown in pharmacomap B for risperidone, risperidone activated the prelimbic (PL), orbital (ORB), piriform (PIR) and gustatory (GU) cortices, the dorsal and ventral CP, ACB, claustrum (CLA), and superior colliculus (SC). Reciprocal connections between cortex and CLA, unidirectional connections from cortex to CP and ACB, and a multisynaptic pathway between SC and CP are indicated. Cortical areas (left) and brainstem areas (right) are grouped in dashed ovals. As shown in pharmacomap C for aripiprazole, aripiprazole activated a partially overlapping pattern, with more cortical areas, including prominent activation of auditory association and entorhinal areas. Parts of the amygdala (AMG), hippocampal formation (HF), and midline thalamus (PVT and RE) also showed activation. A subset of cortical areas is repeated at lower left, in association with the hippocampal formation. It is noted that SC, an important input structure to the striatum via indirect pathways, was activated by both risperidone and aripiprazole. The CP and ACB, highlighted in gray, are common structures that were activated by all three drugs.

TABLE 3

| | Halop | Risper | Aripip |
|---|---|---|---|
| Anterior cingulate cortex (ACAd) | up | 0 | up |
| Anterior cingulate cortex (ACAv) | 0 | 0 | up |
| Basal, Central, Cortic. Amydgala (AMG) | 0 | 0 | up |
| Bed nucleus of Stria Terminalis (BST) | 0 | 0 | 0 |
| Caudoputamen (CP, dorsolateral) | up | up | up |
| Caudoputamen (CP, ventrolateral) | up | up | 0 |
| Caudate-putamen (CP, dorso-medial) | up | 0 | 0 |
| Caudate-putamen (CP, ventro-medial) | up | 0 | 0 |
| Central medial thalamic nucleus | 0 | 0 | up |
| Claustrum (CLA) | 0 | up | up |
| Dorsomedial hypoth. (HYP) | up | 0 | 0 |
| Gostatory Cx (GU) | 0 | up | up |
| Hippocampus: CA1, CA2, CA3 (HF) | 0 | 0 | up |
| Hippocampus: DG | 0 | 0 | down |
| Hypothalamus, ventromedial nuclei | 0 | down | 0 |
| Infralimbic cortex (IL) | up | 0 | up |
| Insular area, agranular | 0 | 0 | up |
| Lateral Septum (LS) | up | 0 | 0 |
| Lateral Spetal Nucl, dorsal part | 0 | 0 | up |
| Motor area, primary (MOp) | 0 | 0 | up |
| Motor area, secondary (MOs) | 0 | 0 | up |
| Midline Thalamus (Nucl Reuniens, RE) | 0 | 0 | up |
| Midline Thalamus (PVT) | 0 | 0 | up |
| Nucleus Accumbens (ACB), core | up | up | up |
| Nucleus Accumbens (ACB), shell | up | 0 | up |
| Nucleus Accumbens (ACB), whole | up | 0 | 0 |
| Olfactory tubercle (OT) | up | 0 | up |
| Orbital cx (ORB), lateral | 0 | up | up |
| Orbital cx (ORB), medial | up | up | up |
| Orbital cortex ventral (VO) | 0 | up | 0 |
| Piriform cx (PIR) | 0 | up | 0 |
| Prelimbic cx (PL) | up | down | up |
| Red nucleus (RN) | 0 | 0 | down |
| RSPd, v, d and agl | 0 | 0 | up |
| Somatosensory cx, primary | 0 | 0 | up |
| Superior Colliculus (SC) | 0 | 0 | up |
| Striatum, ventro-lateral | 0 | 0 | up |
| Temporal cortex | 0 | 0 | up |
| Ventromedial Thalamus (caudally) | 0 | 0 | up |
| Zona Inserta | 0 | 0 | up |

Thus, this example demonstrates that pharmacomaps can be generated and compared to obtain information regarding the activation of different areas of the brain at cellular resolution. In addition, this example demonstrates that the pharmacomaps can be used to differentiate the three different drugs.

In particular, the data of drug-evoked c-Fos activation presented in this example demonstrate that the methods described herein can differentiate between three different antipsychotics (one typical and two atypical). Drug-evoked patterns were reflected on both the number of activated brain regions and the strength of activation within the regions. Mapping brainwide c-Fos induction using the methods described in this example revealed unique brain activity patterns, showing distinct and rich patterns of brain activation, for each of the three drugs used.

These data suggest that fingerprint-like signatures of drug-induced neuronal activity reflect the effects of the drug on the brain and behavior, and thus, such signatures may be correlated with clinical effects.

### 6.10 Example 10

### Generation of Haloperidol Dose-Response Pharmacomaps

This example demonstrates that pharmacomaps can be generated for the same drug at different doses, and that those pharmacomaps can be compared to differentiate the brain activation at different doses.

Drugs have different effects and side effects at different dosages. In order to test whether pharmacomaps are able to reveal dose-dependent drug effects in the brain, the brain activation patterns evoked by the typical antipsychotic haloperidol at three dosages: 0.05 (low), 0.25 (medium) and 1.0 (high) mg/kg was compared. FIG. 48 illustrates pharmacomaps for different dosages of haloperidol. The comparison of pharmacomaps (e.g., A, B, C corresponding to the three dosages respectively) revealed clear differences, with increasing numbers of activated areas observed with increasing dosage. As shown in pharmacomap A, 0.05 mg/kg haloperidol activated dorsomedial hypothalamus (HYP), ACB, and CP. For CP, activation was limited to the dorsal and ventral subdivision. As shown in pharmacomap B, 0.25 mg/kg haloperidol activated the same structures as shown in pharmacomap A, plus OT, LS and PL. Larger portions of ACB and CP were involved. As shown in pharmacomap C, 1.0 mg/kg haloperidol showed a more widespread activation, including, in addition, prelimbic (PL), infralimbic (IL), and lateral entorhinal (ENT) areas, BST, central amygdala (CEA) and PVT. Larger portions of the ACB and CP, compared to the two lower doses, were activated. In addition, within the commonly activated regions (caudate putamen and nucleus accumbens), the strength of c-Fos induction significantly increased with increasing dosage (data not shown).

Thus, the data of drug-evoked c-Fos activation presented in this example demonstrate that the methods described herein can differentiate between three dosages of a single typical antipsychotic. Drug-evoked patterns were reflected on both the number of activated brain regions and the strength of activation within the regions. In particular, both the strength of c-Fos induction and the numbers of activated areas were increased with increasing dosage of haloperidol used. Thus, these data show that pharmacomaps are able to reveal dose-dependent drug effects in the brain.

### 6.11 Example 11

### Generation of a Comprehensive Database of Pharmacomaps

FIG. 49 illustrates an example of generating a comprehensive database of pharmacomaps for predicting therapeutic and adverse effects of drugs, e.g., new drugs. Pharmacomaps of a plurality of drugs (e.g., psychiatric drugs) may be generated and stored in a comprehensive database (e.g., an animal-to-human database). Information related to therapeutic or adverse effects of the plurality of drugs is compiled and stored in the database. A pharmacomap of a new drug is generated and stored in the database. Then, the pharmacomap of the new drug is compared to the pharmacomaps of the plurality of drugs. Based on the comparison, therapeutic or adverse effects of the new drug can be predicted. For example, the database is an animal-to-human (A2H) database including pharmacomaps of a large number of widely used psychiatric medications (e.g., 61 most representative neuropsychiatric drugs) generated from neural activation data of mouse brains. The A2H database links the pharmacomaps of the psychiatric medications to human clinical indications and adverse effects, and thus can be used for predicting human clinical outcomes of new drugs.

As an example, the A2H database may be generated for 20 psychiatric medications with distinct clinical effects and side-effect profiles, as determined from public documents (e.g., the Side Effect Resource (SIDER) database (Kuhn et al., Molecular systems biology 6:343 (2010))). The twenty psychiatric medications can be divided into 10 groups, 1) typical antipsychotics: haloperidol and pimozide; 2) atypical antipsychotics: paliperidone and olanzapine; 3) SSRI antidepressants: sertraline and paroxetine; 4) tricyclic antidepressants: doxepin and clomipramine; 5) MAOI antidepressants: isocarboxazid and phenelzine; 6) tetracyclic antidepressants: mirtazapine and maprotiline; 7) SNRI antidepressants: venlafaxine and desvenlafaxine; 8) anxiolytics: clonazepam and chlordiazepoxide; 9) ADHD medication: methylphenidate and methamphetamine; and 10) Mood stabilizing and anticonvulsant medication: gabapentin and carbamazepine. The drugs' doses are chosen to correspond to clinically relevant doses based on existing literature. Pharmacomaps for these drugs are generated as described above in Example 8. Each of the 20 drugs is screened in five mice, and each drug group is compared to saline control groups and the other drugs.

For example, pairs of drugs both across and within the ten groups of drugs listed above are compared. For every pair of drugs, a list of brain regions is generated to show statistically significant responses, controlled by a failure discovery rate (FDR), by either drug (union) and by both drugs (overlap). The similarity between pharmacomaps is measured by evaluating the fractional overlap (Jaccard similarity coefficient) equal to overlap/union×100%. For non-overlapping/identical responses for two drugs, this measure is equal to 0/100% respectively. Bootstrap methods are used to test whether the values of overlap observed are statistically significant.

Adding pharmacomaps and clinical effects and side-effects of known drugs to the database will continuously increase the value of the A2H database for preclinical drug screening. For example, a comprehensive set of 61 medications from the NIMH database can be screened, including the following.
1) typical antipsychotics: chlorpromazine, fluphenazine, haloperidol, ioxapine, molindone, perphenazine, pimozide, thioridazine, thiothixene, trifluoperazine;
2) atypical antipsychotics: aripiprazole, clozapine, olanzapine, paliperidone, quetiapine, risperidone, ziprasidone;
3) SSRI antidepressants: citalopram, fluoxetine, fluvoxamine, paroxetine, sertraline;
(4) tricyclic antidepressants: amitriptyline, amoxapine, clomipramine, desipramine, doxepin, imipramine, nortriptyline, protriptyline, trimipramine;
(5) MAOI antidepressants: tranylcypromine, phenelzine, isocarboxazid;
(6) SNRI antidepressants: desvenlafaxine, duloxetine, venlafaxine;
(7) tetracyclic antidepressants: maprotiline, mirtazapine;
(8) other antidepressants: bupropion, trazodone, selegiline;
(9) benzodiazepine anxiolytics: alprazolam, chlordiazepoxide, clonazepam, iorazepam, oxazepam, diazepam;
(10) other anxiolytics: buspirone;
(11) Mood stabilizing and anticonvulsants: carbamazepine, gabapentin, lamotrigine, lithium carbonate, oxcarbazepine, topiramate, valproic acid;
(12) ADHD medications: amphetamine, atomoxetine, guanfacine, methamphetamine HCl, methylphenidate.

Each of the 61 drugs is screened at two dosages, one that corresponds to the clinically relevant dose used in humans and a high dose (above the therapeutic range) that is known to cause significant side effects in humans. The purpose of the supratherapeutic dose is to generate pharmacomaps representing unacceptable side effects. These maps will be complemented by pharmacomaps of drugs which failed clinical trials so that the A2H database includes both acceptable and unacceptable pharmacomaps growing in parallel. To create the A2H database, the pharmacomap data is linked to the data of the clinical effects and side effects available for these drugs from public documents, such as the SIDER database which provides incidence data for more than 800 side-effects (Kuhn et al., Molecular systems biology 6:343 (2010)). Beyond laying the groundwork for making “go/no-go” decisions regarding clinical trials, these data lay the groundwork for associating clinical effects and side effects with neuronal activation at an unprecedented resolution.

As an example, among the 61 drugs, 20 psychiatric medications with distinct clinical effects and side-effect profiles can be screened at the high dose (above the therapeutic range) and the remaining 41 drugs at both the clinically relevant dose used in humans and the high dose (above the therapeutic range). The two dosages for each drug can be curated from the existing extensive literature on behavioral drug testing in rodent models (for example, see the dosages studies of several antipsychotics (Kelly et al., J Neurosci 18, 3470-3479, (1998); Natesan et al., Neuropsychopharmacology 31, 1854-1863 (2006); Oka et al., Life sciences 76, 225-237 (2004); Robertson and Fibiger, Neuroscience 46, 315-328 (1992); Simon et al., Eur Neuropsychopharmacol 10, 159-164 (2000); Wan et al., (Brain research 688, 95-104) 1995)). Using 5 brains per group, the total of the screened drug-treated brains are: (1×20+2×41)×5=510. In addition, 4 saline groups (one each 6 months; 20 brains in total) are included to control for any changes in conditions. The total number of brains screened may therefore be 530.

For the purposes of selection of the appropriate drug dosages, the mice can be video-monitored before and after the drug application and their behavior can be scored by an automated behavior analysis software in categories such as rest, walk, groom, hang, rear, drink, eat, etc. The changes in the mouse behaviors can be used to evaluate the drug doses used with respect to the expected clinically relevant side effects, especially for the supra-therapeutic dose ranges. Small modules of drug-induced behavioral changes may be built and used for comparisons of drugs that would be expected to cause similar side-effects in the clinics.

### 6.12 Example 12

### Correlating Mouse Brain Pharmacomaps with Human Clinical Outcomes

The increasing amount of publicly available data about properties of chemical compounds creates opportunities for integrating these data into a predictive model of drug effects. The NIH Molecular Libraries Roadmap Initiative has led to creation of the PubChem repository of chemical compounds (Sayers et al., Nucleic acids research 40, D13-25 (2012)). Databases such as Pubchem, BioAssays, and ChemBank contain information about drug-target interactions (Seiler et al., Nucleic acids research 36, D351-359 (2008)) and cellular phenotypes induced by exposure to small molecules. The SIDER database contains detailed information about drugs' side effects (Kuhn et al., Molecular systems biology 6, 343 (2010)) that are predictive of drug-target interactions (Campillos et al., Science 321, 263-266 (2008)).

The structure of the adverse effects (AEs) data from the SIDER database (Kuhn et al., Molecular systems biology 6:343 (2010)) which contains more than 800 drugs are analyzed for correlating pharmacomaps with clinical data. For the 61 psychiatric drugs as described in Example 11, the SIDER database contains 834 AEs and 56 indications, with each compound on average associated with approximately 130 AEs and approximately 3 indications. When represented as a 61-by-834 binary table, AEs can be compared between pairs of compounds to yield a distance matrix, indicating how similar the AE profiles are between the two drugs in the pair.

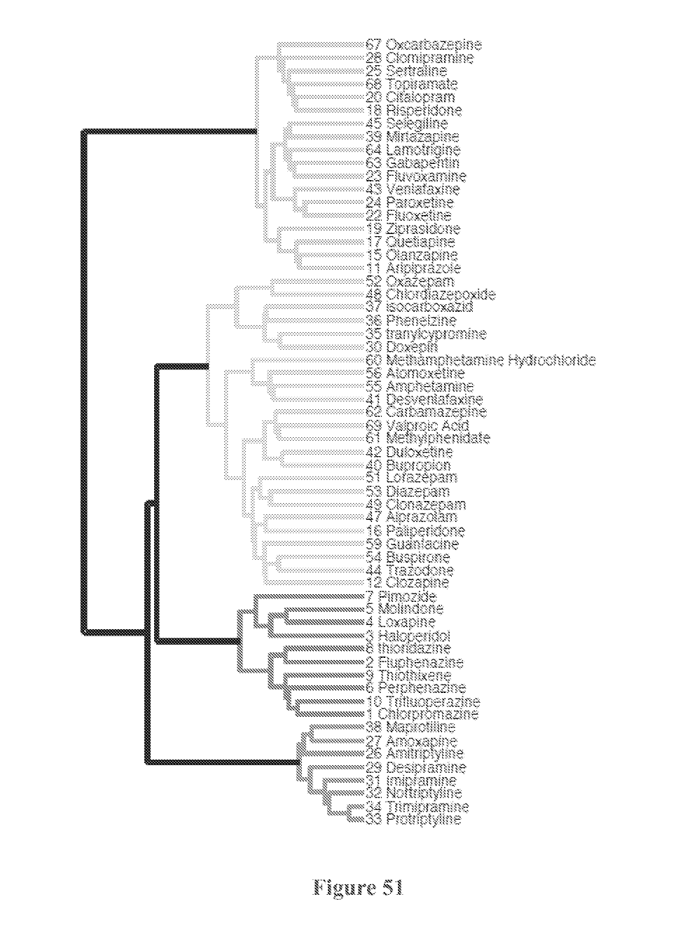

A pharmacomap of a new drug can be compared to those of known drugs to predict AE and/or indication(s) for the new drug, as shown in FIG. 52. To determine how predictive pharmacomaps are, Principal Component Analysis (PCA) of adverse effects and indications for drugs were performed first. FIG. 50 illustrates example Principal Component Analysis (PCA) of adverse effects and indications for drugs, and FIG. 51 illustrates example representation of adverse effects for drugs.

Pairwise distances were analyzed by PCA as shown in FIG. 50 and were clustered using agglomerative hierarchical trees as shown in FIG. 51. It is evident that compounds with similar indications clustered together in both PCA space and on hierarchical trees. In FIG. 50, four major groups of medications are illustrated. Typical antipsychotics (+) and tricyclic antidepressants (V) clustered as separate groups according to their AEs. Anti-anxiety medicines (*) formed a cluster with ADHD drugs (o) and other types of antidepressants (other triangles). Atypical antipsychotics (x), for the most part, clustered with mood stabilizing and anticonvulsant medications (dots) and SSRI antidepressants (squares). The compounds' clustering is also shown in FIG. 51. Even within diagnostic class, however, each molecule exhibited a distinct AE profile, providing ample variability against which to correlate the expected diversity of pharmacomaps.

FIG. 52 illustrates an example of data measuring similarity in pharmacomaps of haloperidol, risperidone, and aripiprazole. HAL, RISP, and ARIP stand for Haloperidol, Risperidone, and Aripiprazole respectively. As shown in FIG. 52, Pharmacomaps for ARIP and RISP were more similar than for ARIP-HAL and RISPHAL pairs. Similarities in pharmacomaps therefore reflected similarities in AE/indications, as indicated for these classes of compounds. For example, to determine similarity between activities, the fraction of brain regions that were co-affected by two drugs (intersection/union×100%) were compared. The fraction of common effects between pairs of drugs was determined to define similarities in AE/indications. Thus, the pharmacomap of a new drug can be compared to those of known drugs to predict AE and/or indication(s) for the new drug.

To extend the prediction analyses to the 61-drug dataset, the pairwise distances between drugs in terms of their pharmacomaps and similarities in AEs were compared. If pharmacomaps are predictive or causal of AEs and indications, similar activity patterns are to yield similar AEs. For the 61 drugs noted above, 1830 pairwise similarities in both in pharmacomap space and in AE space were determined. The Pearson correlation coefficient between pairwise similarities was computed to describe the degree of relationship between these two spaces. High correlation coefficient implied that pharmacomaps are predictive of AEs. Clinical indications were included into the analysis of pairwise distances to more fully explore the effects of drugs. The correlation between pairwise distances in pharmacomap space and AE+indication space was better than in AE space alone, because indications can be related to some features of pharmacomaps leading to an additional contribution to correlations. The advantage of the comparison of pairwise distances between the space of neural responses and AEs is that such an analysis does not involve building a model of mapping between these two spaces. A predictive model for the mapping between pharmacomap space and AE space can be built. Each AE is treated as an independent variable. AEs for which frequency information is not available in the SIDER database is treated as binary variables equal to 1/0 if an AE is present/absent.

For example, the 61 drugs can be classified into those have the ones that have or do not have the given AE. Because the pharmacomaps are represented by cell counts in >80 brain regions for each of the 61 drugs, in building the predictor for each AE, the number of parameters (>80) is larger than the number of data points (61). A greedy sparsification algorithm (Koulakov et al., Frontiers in systems neuroscience 5, 65 (2011); Haddad et al., Nature methods 5:425-429 (2008); Saito et al., Science signaling 2, ra9 (2009)) can be used to reduce the number of parameters by removing from consideration brain areas that are not strong predictors for each AE, and avoid overfitting. The greedy sparsification algorithm starts by going through all of the brain regions one-by-one and building predictors on the basis of a single brain region. After the best brain region for a particular AE is found, the second brain region is selected that maximizes the accuracy of prediction. The greedy recruitment is stopped when substantially low error rate or high correlation between predictions and data are achieved. This analysis allows to dramatically reduce the number of parameters needed for an accurate prediction (Koulakov et al, Frontiers in systems neuroscience 5:65 (2011)).

A jackknife method (Koulakov et al., Frontiers in systems neuroscience 5: 65 (2011); Saito et al., Science signaling 2, ra9 (2009)) can be used to validate the quality of predictor in these conditions. For example, one drug is removed from the dataset completely. The predictor is built of the basis of responses to other drugs, and the prediction is generated for the drug that has been removed. This procedure is then repeated for every compound in the dataset. Predictions for all of the compounds are then compared to the actual values of AE. The quality of prediction will be judged on the basis of error rate and Pearson correlation coefficient.

To implement classification itself, several methods may be used, such as linear discriminate analysis or Fisher's linear discriminant (Raudys, Statistical and neural classifiers: an integrated approach to design, Springer Verlag (2001)), the Bayes optimal predictor within quadratic discriminant analysis (Raudys, Statistical and neural classifiers: an integrated approach to design, Springer Verlag (2001)), and support vector machines (Cristianini et al., An introduction to support Vector Machines: and other kernel-based-learning methods, Cambridge Univ Pr (2000)). Different types of predictors can be compared on the basis of error rate using the jackknife method described above.

In addition to predicting AEs, pharmacomaps can be used to build a predictive model for drug indications. The set of indications for each drug is available from SIDER database. Using validation with the jackknife method, the quality of prediction can be determined by computing prediction error. Because in the jackknife analysis every drug is treated as de novo prediction, prediction errors for drugs within/outside of included categories can be compared. This test may determine whether mouse brain activity patterns can generalize across indications for different classes of medications. Such predictive algorithms may be useful in preclinical drug development, since often a drug being developed for a particular indication turns out to have uses beyond that indication. The predictive algorithms may provide a way to anticipate these additional indications.

### INCORPORATION BY REFERENCE

Various references such as patents, patent applications, and publications are cited herein, the disclosures of which are hereby incorporated by reference herein in their entireties.

1. A method of predicting the therapeutic effect or toxicity effect of a test compound comprising:
(a) administering the test compound to a transgenic animal, wherein the transgenic animal comprises a genetic regulatory region that controls expression of a fluorescent reporter gene sequence;
(b) harvesting a tissue of the transgenic animal;
(c) imaging the harvested tissue using an imaging technique that provides single cell resolution of cells expressing the fluorescent reporter gene sequence in the tissue, thereby generating a pharmacomap of the test compound; and
(d) comparing the pharmacomap in (c) to that of a pharmacomap of a reference compound, wherein the reference compound has a known therapeutic or toxicity effect, thereby predicting the therapeutic effect or toxicity effect of the test compound based on the similarity of the pharmacomaps.

2. The method of claim 1, wherein the transgenic animal is a mouse.

3. The method of claim 1, wherein the tissue is brain, kidney, liver, pancreas, stomach or heart tissue.

4. The method of claim 3, wherein the tissue is brain tissue.

5. The method of claim 4, wherein the brain tissue is whole brain.

6. The method of claim 3, wherein the tissue is liver tissue.

7. The method of claim 6, wherein the liver tissue is whole liver.

8. The method of claim 1, wherein step (b) comprises harvesting two tissues.

9. The method of claim 8, wherein the tissues are selected from brain, kidney, liver, pancreas, stomach and heart tissue.

10. The method of claim 9, wherein the two tissues are brain tissue and liver tissue.

11. The method of claim 1, wherein the imaging technique is serial two-photon tomography.

12. The method of claim 1, wherein the genetic regulatory region is a genetic regulatory region of an immediate early gene.

13. The method of claim 12, wherein the genetic regulatory region is that of an immediate early gene that is activated within 30 minutes after a stimulus.

14. The method of claim 12, wherein the immediate early gene is c-fos, FosB, delta FosB, c-jun, CREB, CREM, zif/268, tPA, Rheb, RGS2, CPG16, COX-2, Narp, BDNF, CPG15, Arcadlin, Homer-1a, CPG2, or Arc.

15. The method of claim 14, wherein the immediate early gene is c-fos.

16. The method of claim 14, wherein the immediate early gene is Arc.

17. The method of claim 1, wherein the genetic regulatory region is that of a gene that is activated downstream of an immediate early gene.

18. The method of claim 1, wherein the genetic regulatory region is that of a gene that is activated more than 30 minutes after a stimulus.

19. The method of claim 1, wherein the genetic regulatory region is that of a gene that is activated more than 1 hour after a stimulus.

20. The method of claim 1, wherein the reporter gene sequence encodes green fluorescent protein (GFP).

21. The method of claim 1, wherein the comparing step comprises statistical significance analyses.

22. The method of claim 1, which is used for predicting therapeutic effect of the test compound, and wherein the reference compound has a known therapeutic effect.

23. The method of claim 22, wherein the reference compound has a known therapeutic effect in a human.

24. The method of claim 1, which is used for predicting toxicity effect of the test compound, and wherein the reference compound has a known toxicity effect.

25. The method of claim 24, wherein the reference compound has a known toxicity effect in a human.

26. The method of claim 1, wherein the reference compound is a drug that is used for treating a brain disorder.

27. The method of claim 1, wherein the pharmacomap in (d) is present in a database comprising a plurality of reference compound pharmacomaps.

28. The method of claim 1, wherein the method is repeated with a plurality of test compounds.

29. The method of claim 28, wherein the pharmacomaps obtained for each of the test compounds are compiled into a single database.

30. The method of claim 28, wherein the data obtained for each of the test compounds in the comparing step are compiled into a single database.

31. The method of claim 1 further comprising: using a machine learning algorithm to detect activated cells associated with the imaged tissue.

32. The method of claim 31, wherein the machine learning algorithm comprises a convolutional neural network algorithm.

33. The method of claim 1, wherein the pharmacomap is of an entire brain of the transgenic animal.

34. The method of claim 1 further comprising:
warping of the imaged harvested tissue into a volume of continuous tissue space; performing voxelization of the continuous tissue space to generate discrete digitization of the continuous tissue space;
using statistical techniques upon the discrete digitization to identify areas of significant differences between control and drug-activated tissue areas; and
using anatomical segmentation to assign the significant differences to tissue regions and to determine numbers of activated cells for one or more of the tissue regions
wherein the determined number of activated cells is used in said comparing of the pharmacomap in (c) to that of the pharmacomap of a reference compound.

35. A method of predicting the therapeutic effect or toxicity effect of a test compound comprising:
(a) administering the test compound to a transgenic animal, wherein the transgenic animal comprises a genetic regulatory region that controls expression of a fluorescent reporter gene sequence;
(b) harvesting a tissue of the transgenic animal;
(c) imaging the harvested tissue using an imaging technique that provides single cell resolution of cells expressing the fluorescent reporter gene sequence in the tissue, thereby generating a pharmacomap of the test compound; and
(d) comparing the pharmacomap in (c) to that of a database of pharmacomaps of reference compounds, wherein the reference compounds have known therapeutic or toxicity effect, thereby predicting the therapeutic effect or toxicity effect of the test compound based on the similarity of the pharmacomaps.

36. A method of generating a pharmacomap, comprising:
(a) administering a compound to a transgenic animal comprising a genetic regulatory region that controls expression of a fluorescent reporter gene sequence;
(b) harvesting a tissue of the transgenic animal; and
(c) imaging the harvested tissue using an imaging technique that provides single cell resolution of cells expressing the fluorescent reporter gene sequence in the tissue, thereby generating a pharmacomap of the compound.

37. The method of claim 36, wherein the compound is a reference compound having a known therapeutic or toxicity effect.

38. A method of generating a pharmacomap of a test compound for predicting therapeutic effects or toxicity effects of the test compound, wherein the test compound is administered to a transgenic animal that includes a genetic regulatory region to control expression of a fluorescent reporter gene sequence, wherein a tissue of the transgenic animal is harvested, the method comprising:
imaging the harvested tissue using an imaging technique that provides single cell resolution of cells expressing the fluorescent reporter gene sequence in the tissue;
identifying, by use of one or more data processors, cells that are activated in response to the test compound using a machine learning algorithm;
generating a representation, by use of the one or more data processors, of the identified cells into a volume of continuous tissue space;
performing, by use of the one or more data processors, statistical techniques to identify regions of significant differences based on a comparison of the generated representation of the identified cells of the harvested tissue and a representation of cells of a control tissue; and
generating, by use of the one or more data processors, a pharmacomap of the test compound based on the identified regions of significant differences to identify anatomical tissue regions that are activated in response to the test compound for predicting therapeutic effects or toxicity effects of the test compound.

39. The method of claim 38, wherein the step of generating a representation of the identified cells into a volume of continuous tissue space comprises:
warping of the tissue images into a standard volume of continuous tissue space to register information associated with the identified cells within the continuous tissue space; and
performing voxelization of the continuous tissue space to generate discrete digitization of the continuous tissue space.

40. The method of claim 39, wherein the pharmacomap is stored in a computer-readable storage medium;
wherein the computer-readable storage medium includes a storage area for storing voxel data that is representative of the continuous tissue space;
wherein the computer-readable storage medium includes data fields for storing pharmacomap data that identifies the activated anatomical tissue regions in the tissue space represented by the voxel data;
wherein an activated anatomical tissue region comprises one or more voxels, and a voxel is representative of a tissue region having one or more cells that are activated in response to the test compound.

41. The method of claim 40, wherein the computer-readable storage medium is a database stored in a non-transitory storage medium, or a memory device.

42. The method of claim 40, wherein the computer-readable storage medium includes pharmacomap data of one or more reference compounds which is associated with therapeutic effects or toxicity effects of the reference compounds upon particular regions of tissue; wherein the pharmacomap data of the test compound is compared with the pharmacomap data of the one or more of the reference compounds in order to predict the therapeutic effects or toxicity effects of the test compound.

43. The method of claim 38, wherein the step of generating a pharmacomap of the test compound includes performing an anatomical segmentation of the identified regions of significant differences.

44. The method of claim 38, wherein the machine learning algorithm includes one of the following: a convolutional neural network algorithm, support vector machines, random forest classifiers, and boosting classifiers.

45. The method of claim 38, wherein the statistical techniques include a negative binomial regression technique.

46. The method of claim 38, wherein the statistical techniques include one or more t-tests.

47. The method of claim 38, wherein the statistical techniques include a random field theory technique.

48. The method of claim 38, wherein the imaging technique includes one of the following: a serial two-photon tomography, Allen institute serial microscopy, all-optical histology, robotized wide-field fluorescence microscopy, light-sheet fluorescence microscopy, OCPI light-sheet, and micro-optical sectioning tomography.

49. A method of predicting therapeutic effects or toxicity effects of a test compound, wherein the test compound is administered to a transgenic animal that includes a genetic regulatory region to control expression of a fluorescent reporter gene sequence, wherein a tissue of the transgenic animal is harvested, the method comprising:
generating, by use of one or more data processors, a pharmacomap of the test compound by identifying anatomical tissue regions in the harvested tissue that are activated in response to the test compound, wherein the pharmacomap includes a representation of a tissue space of the harvested tissue, and includes pharmacomap information that identifies the activated anatomical tissue regions in the tissue space;
comparing, by use of the one or more data processors, the pharmacomap of the test compound to a predetermined pharmacomap of a reference compound, wherein the reference compound has a known therapeutic or toxicity effect that correlates to the pharmacomap of the reference compound; and
predicting the therapeutic effects or toxicity effects of the test compound based on the comparison of the pharmacomaps of the test compound and the reference compound.

50. The method of claim 49, wherein the step of predicting the therapeutic effects or toxicity effects of the test compound includes:
generating a correlation matrix of the reference compound between the known therapeutic or toxicity effect of the reference compound and the pharmacomap of the reference compound.

51. The method of claim 49, wherein the representation of the tissue space of the harvested tissue includes generation of a three-dimensional image of the harvested tissue, warping of the three-dimensional image into a standard volume of the tissue space, and voxelization of the tissue space to generate discrete digitization of the tissue space.

52. The method of claim 51, wherein an activated anatomical tissue region comprises one or more voxels; and wherein a voxel includes one or more cells that are activated in response to the test compound.
